(19) United States
(12) Patent Application Publication
(10) Pub. No.: US 2026/0007612 A1
(43) Pub. Date: Jan. 8, 2026

(54) Novel Ionizable Cationic Lipids

(71) Applicant: Vivasor, Inc., San Diego, CA (US)

(72) Inventors: Peng Wang, San Diego, CA (US); Hua Wang, San Diego, CA (US); Haotian Sun, San Diego, CA (US); Ying Zeng, San Diego, CA (US); Hui Xie, San Diego, CA (US)

(73) Assignee: Vivasor, Inc., San DIego, CA (US)

(21) Appl. No.: 18/841,178

(22) PCT Filed: Feb. 23, 2023
(86) PCT No.: PCT/US2023/063122

§ 371 Date: Aug. 23, 2024

Related U.S. Application Data
(60) Provisional application No. 63/313,648, filed on Feb. 24, 2022.

(51) Int. Cl. A61K 9/51; C07C 219/06; C07D 295/13; C07D 317/28; C07D 339/08

(52) U.S. Cl. CPC A61K 9/5123; C07C 219/06; C07D 295/13; C07D 317/28; C07D 339/08

(57) ABSTRACT

Provided herein, inter alia, novel ionizable cationic lipids, lipid nanoparticles comprising the novel ionizable cationic lipids and methods of using said nanoparticles.

### CROSS-REFERENCE TO RELATED APPLICATIONS

This application claims the benefit of priority of U.S. Provisional Patent Application No. 63/313,648, filed Feb. 24, 2022, which is incorporated herein by reference in its entirety for all purposes.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### TECHNICAL FIELD

The present disclosure provides novel ionizable cationic lipids which can be used to form lipid nanoparticles, e.g., to facilitate intracellular payload delivery (e.g., of mRNA, DNA, siRNA, oligonucleotides, amplified RNA, plasmids, ribozymes, aptamers, etc.).

### INTRODUCTION AND SUMMARY

Many macromolecules, such as nucleic acid molecules, cannot easily cross cell membranes because of their size, charge, and/or hydrophilicity. Delivery has therefore been one of the major challenges for such therapeutics, e.g., antisense payloads and mRNA technology. A formulation containing molecules not only must (1) protect the payload from enzymatic and non-enzymatic degradation and (2) provide appropriate biodistribution of the formulation, but also (3) allow cellular uptake or internalization of the formulation and (4) facilitate delivery of the nucleic acid payload to the cytoplasm of the cell.

Cationic lipids are amphiphilic molecules that generally contain a lipophilic region containing one or more hydrocarbon groups, and a hydrophilic region containing at least one polar head group that is positively charged or ionizable. Cationic lipids facilitate entry of macromolecules, such as nucleic acids, into the cytoplasm through the cell plasma membrane by forming a positively charged (total charge) complex with macromolecules. This process, performed in vitro and in vivo, is known as transfection.

Typically, cationic lipids are used alone or in combination with other lipids, e.g., phospholipids and/or neutral lipids such as cholesterol, and/or polyethylene glycol (PEG)-functionalized lipids (PEG-lipids) (Meng, C. et al., 2021, Adv. Ther. 4:2000099). These lipids can improve nanoparticle properties, such as particle stability, delivery efficacy, tolerability and biodistribution (Hajj, K. A. et al., 2017, Nat. Rev. Mater. 2:17056; Kim, J. et al., 2021, Adv. Drug Deliv. Rev. 170:83-112). For example, 1,2-distearoyl-sn-glycero-3-phosphocholine (DSPC), a phosphatidylcholine with saturated tails, has a melting temperature of ˜54° C. and a cylindrical geometry that allows DSPC molecules to form a lamellar phase, which stabilizes the structure of lipid nanoparticles (Koltover, I. et al., 1998, Science 281:78-81). A combination of cationic lipids and neutral lipids can easily form a vesicle that contains an aligned lipid bilayer. Vesicles and liposomes formed by cationic lipids either alone or in combination with neutral lipids have many positive charges on the surface, and, with these charges, can form a complex with polynucleotides or other anionic molecules such as negatively charged proteins. The remaining total cationic charge on the surface of a polynucleotide/cationic lipid/neutral lipid complex can cause strong interaction with the cell membrane, mainly with the negative charge on the surface of the cell membrane.

Many different types of cationic lipids have been synthesized for use in transfection and are currently commercially available. Such cationic lipids include, for example, Lipofectin, Lipofectin ACE, Lipofect AMINE, Transfeactam, and DOTAP.

Despite the abundance of cationic lipids, there is still a need in the art for improved lipid-therapeutic nucleic acid compositions that are suitable for general therapeutic use. Preferably, these compositions would encapsulate nucleic acids with high efficiency, have high drug:lipid ratios, protect the encapsulated nucleic acid from degradation and clearance in serum, be suitable for systemic delivery, and/or provide intracellular delivery of the encapsulated nucleic acid. In addition, these nucleic acid-lipid particles should preferably be well-tolerated and/or provide an adequate therapeutic index, e.g., such that patient treatment at an effective dose of the nucleic acid is not associated with significant toxicity and/or risk to the patient.

The present disclosure aims to meet one or more of these needs, provide other benefits, or at least provide the public with a useful choice. Provided herein are novel ionizable cationic lipids, e.g., with enhanced efficiency of in vivo delivery.

In an aspect, provided herein is a cationic lipid of formula (I)

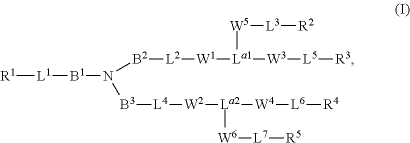

or a pharmaceutically acceptable salt, solvate, hydrate, stereoisomer, or prodrug thereof. R¹ is H, —OR^{1A}, —YOR^{1A}, —NR^{1A}R^{1B}, —YNR^{1A}R^{1B}, —SR^{1A}, —YSR^{1A}, —(C═O)R^{1A}, —Y(C═O)R^{1A}, —(C═O)OR^{1A}, —Y(C═O)OR^{1A}, —O(C═O)R^{1A}, —YO(C═O)R^{1A}, —O(C═O)OR^{1A}, —YO(C═O)OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
Y is substituted or unsubstituted C₀-C₁₂ alkylene or substituted or unsubstituted 0- to 12-membered heteroalkylene.
R² is H, —OR^{2A}, —SR^{2A}, —(C═O)R^{2A}, —(C═O)OR^{2A}, —O(C═O)R^{2A}, —O(C═O)OR^{2A}, —(C═O)NHR^{2A}, —NH(C═O)R^{2A}, substituted or unsubstituted alkyl, or substituted or unsubstituted heteroalkyl.
R³ is H, —OR^{3A}, —SR^{3A}, —(C═O)R^{3A}, —(C═O)OR^{3A}, —O(C═O)R^{3A}, —O(C═O)OR^{3A}, —(C═O)NHR^{3A}, —NH(C═O)R^{3A}, substituted or unsubstituted alkyl, or substituted or unsubstituted heteroalkyl.
R⁴ is H, —OR^{4A}, —SR^{4A}, —(C═O)R^{4A}, —(C═O)OR^{4A}, —O(C═O)R^{4A}, —O(C═O)OR^{4A}, —(C═O)NHR^{4A}, —NH(C═O)R^{4A}, substituted or unsubstituted alkyl, or substituted or unsubstituted heteroalkyl.
R⁵ is H, —OR^{5A}, —SR^{5A}, —(C═O)R^{5A}, —(C═O)OR^{5A}, —O(C═O)R^{5A}, —O(C═O)OR^{5A}, —(C═O)NHR^{5A}, —NH(C═O)R^{5A}, substituted or unsubstituted alkyl, or substituted or unsubstituted heteroalkyl.
B¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
B² and B³ are each independently a bond, substituted or unsubstituted alkylene, or substituted or unsubstituted heteroalkylene.
L¹ is a bond, —O(C═O)—, —(C═O)O—, —O(C═O)O—, —C(═O)—, —O—, —O(CR¹⁰¹R¹⁰²)ₛO—, —S—, —C(═O)S—, —SC(═O)—, —NR¹⁰¹C(═O)—, —C(═O)NR¹⁰¹—, —NR¹⁰¹C(═S)—, —C(═S)NR¹⁰¹—, —NR¹⁰¹C(═O)NR¹⁰²—, —NR¹⁰¹C(═S)NR¹⁰²—, —OC(═O)NR¹⁰¹—, —NR¹⁰¹C(═O)O—, —SC(═O)NR¹⁰¹— or —NR¹⁰¹C(═O)S—.
L² is a bond, —O(C═O)—, —(C═O)O—, —O(C═O)O—, —C(═O)—, —O—, —O(CR²⁰¹R²⁰²)SO—, —S—, —C(═O)S—, —SC(═O)—, —NR²⁰¹C(═O)—, —C(═O)NR²⁰¹—, —NR²⁰¹C(═O)NR²⁰²—, —NR²⁰¹C(═S)—, —C(═S)NR²⁰¹—, —NR²⁰¹C(═S)NR²⁰²—, —OC(═O)NR²⁰¹—, —NR²⁰¹C(═O)O—, —SC(═O)NR²⁰¹— or —NR²⁰¹C(═O)S—.
L³ is a bond, —O(C═O)—, —(C═O)O—, —O(C═O)O—, —C(═O)—, —O—, —O(CR³⁰¹R³⁰²)SO—, —S—, —C(═O)S—, —SC(═O)—, —NR³⁰¹C(═O)—, —C(═O)NR³⁰¹—, —NR³⁰¹C(═O)NR³⁰²—, —NR³⁰¹C(═S)—, —C(═S)NR³⁰¹—, —NR³⁰¹C(═S)NR³⁰²—, —OC(═O)NR³⁰¹—, —NR³⁰¹C(═O)O—, —SC(═O)NR³⁰¹— or —NR³⁰¹C(═O)S—.
L⁴ is a bond, —O(C═O)—, —(C═O)O—, —O(C═O)O—, —C(═O)—, —O—, —O(CR⁴⁰¹R⁴⁰²)ₛO—, —S—, —C(═O)S—, —SC(═O)—, —NR⁴⁰¹C(═O)—, —C(═O)NR⁴⁰¹—, —NR⁴⁰¹C(═O)NR⁴⁰²—, —NR⁴⁰¹C(═S)—, —C(═S)NR⁴⁰¹—, —NR⁴⁰¹C(═S)NR⁴⁰²—, —OC(═O)NR⁴⁰¹—, —NR⁴⁰¹C(═O)O—, —SC(═O)NR⁴⁰¹— or —NR⁴⁰¹C(═O)S—.
L⁵ is a bond, —O(C═O)—, —(C═O)O—, —O(C═O)O—, —C(═O)—, —O—, —O(CR⁵⁰¹R⁵⁰²)SO—, —S—, —C(═O)S—, —SC(═O)—, —NR⁵⁰¹C(═O)—, —C(═O)NR⁵⁰¹—, —NR⁵⁰¹C(═O)NR⁵⁰²—, —NR⁵⁰¹C(═S)—, —C(═S)NR⁵⁰¹—, —NR⁵⁰¹C(═S)NR⁵⁰²—, —OC(═O)NR⁵⁰¹—, —NR⁵⁰¹C(═O)O—, —SC(═O)NR⁵⁰¹— or —NR⁵⁰¹C(═O)S—.
L⁶ is a bond, —O(C═O)—, —(C═O)O—, —O(C═O)O—, —C(═O)—, —O—, —O(CR⁶⁰¹R⁶⁰²)SO—, —S—, —C(═O)S—, —SC(═O)—, —NR⁶⁰¹C(═O)—, —C(═O)NR⁶⁰¹—, —NR⁶⁰¹C(═O)NR⁶⁰²—, —NR⁶⁰¹C(═S)—, —C(═S)NR⁶⁰¹—, —NR⁶⁰¹C(═S)NR⁶⁰²—, —OC(═O)NR⁶⁰¹—, —NR⁶⁰¹C(═O)O—, —SC(═O)NR⁶⁰¹— or —NR⁶⁰¹C(═O)S—.
L⁷ is a bond, —O(C═O)—, —(C═O)O—, —O(C═O)O—, —C(═O)—, —O—, —O(CR⁷⁰¹R⁷⁰²)ₛO—, —S—, —C(═O)S—, —SC(═O)—, —NR⁷⁰¹C(═O)—, —C(═O)NR⁷⁰¹—, —NR⁷⁰¹C(═O)NR⁷⁰²—, —NR⁷⁰¹C(═S)—, —C(═S)NR⁷⁰¹—, —NR⁷⁰¹C(═S)NR⁷⁰²—, —OC(═O)NR⁷⁰¹—, —NR⁷⁰¹C(═O)O—, —SC(═O)NR⁷⁰¹— or —NR⁷⁰¹C(═O)S—.
L^{a1} and L^{a2} are each independently

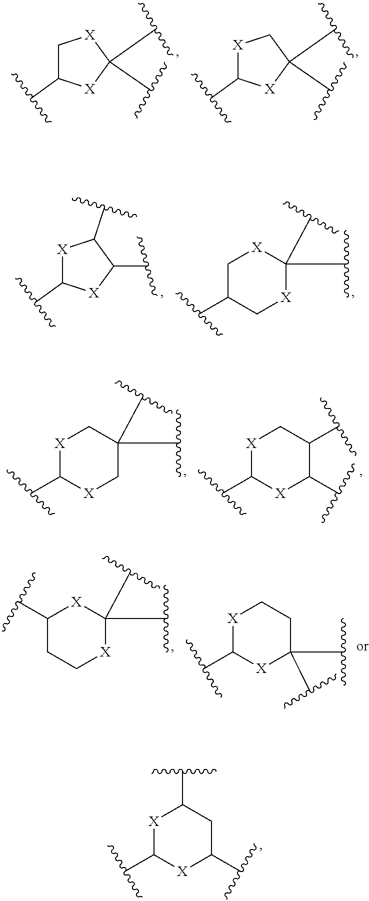

where each X is independently O, S, or CH₂.
W¹, W², W³, W⁴, W⁵, and W⁶ are each independently a bond, substituted or unsubstituted C₁-C₁₂ alkylene, or substituted or unsubstituted 2- to 12-membered heteroalkylene.
Each R^{1A} and R^{1B} is independently H, substituted or unsubstituted C₁-C₁₂ alkyl, or substituted or unsubstituted 2- to 12-membered heteroalkyl.
Each R^{2A}, R^{3A}, R^{4A}, and R^{5A} is independently H, substituted or unsubstituted C₁-C₃₀ alkyl, or substituted or unsubstituted 2- to 30-membered heteroalkyl.
Each R¹⁰¹, R¹⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently H, substituted or unsubstituted C₁-C₁₂ alkyl, or substituted or unsubstituted 2- to 12-membered heteroalkyl.
Each s is independently an integer from 1 to 4.

In an aspect, provided herein is a cationic lipid of formula (II)

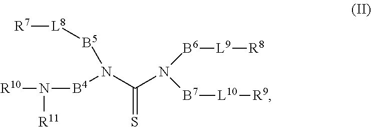

or a pharmaceutically acceptable salt, solvate, hydrate, stereoisomer, or prodrug thereof.
B⁴ is W⁷-L^{a3}-W⁸, where W⁷ and W^{s} are each independently a bond, substituted or unsubstituted alkylene, or substituted or unsubstituted heteroalkylene, and L^{a3} is a bond, —O(C═O)—, —(C═O)O—, —O(C═O)O—, —C(═O)—, —O—, —O(CR^{a31}R^{a32})ₛO—, —S—, —C(═O)S—, —SC(═O)—, —NR^{a31}C(═O)—, —C(═O)NR^{a31}—, —NR^{a31}C(═O)NR^{a32}, —NR^{a31}C(═S)—, —C(═S)NR^{a31}—, —NR^{a31}C(═S)NR^{a32}—, —OC(═O)NR^{a31}—, —NR^{a31}C(═O)O—, —SC(═O)NR^{a31}— or —NR^{a31}C(═O)S—.
R¹⁰ and R¹¹ are each independently H, substituted or unsubstituted alkyl, substituted or unsubstituted heteroalkyl, or R¹⁰ and R¹¹ together with the nitrogen atom to which they are connected form a substituted or unsubstituted heterocycloalkyl or substituted or unsubstituted heteroaryl.
B⁵, B⁶, and B⁷ are each independently a bond, substituted or unsubstituted alkylene, or substituted or unsubstituted heteroalkylene.
L⁸ is a bond, —O(C═O)—, —(C═O)O—, —O(C═O)O—, —C(═O)—, —O—, —O(CR⁸⁰¹R⁸⁰²)SO—, —S—, —C(═O)S—, —SC(═O)—, —NR⁸⁰¹C(═O)—, —C(═O)NR⁸⁰¹—, —NR⁸⁰¹C(═O)NR⁸⁰²—, —NR⁸⁰¹C(═S)—, —C(═S)NR⁸⁰¹—, —NR⁸⁰¹C(═S)NR⁸⁰²—, —OC(═O)NR⁸⁰¹—, —NR⁸⁰¹C(═O)O—, —SC(═O)NR⁸⁰¹ or —NR⁸⁰¹C(═O)S—.
L⁹ is a bond, —O(C═O)—, —(C═O)O—, —O(C═O)O—, —C(═O)—, —O—, —O(CR⁹⁰¹R⁹⁰²)SO—, —S—, —C(═O)S—, —SC(═O)—, —NR⁹⁰¹C(═O)—, —C(═O)NR⁹⁰¹—, —NR⁹⁰¹C(═O)NR⁹⁰²—, —NR⁹⁰¹C(═S)—, —C(═S)NR⁹⁰¹—, —NR⁹⁰¹C(═S)NR⁹⁰²—, —OC(═O)NR⁹⁰¹—, —NR⁹⁰¹C(═O)O—, —SC(═O)NR⁹⁰¹— or —NR⁹⁰¹C(═O)S—.
L¹⁰ is a bond, —O(C═O)—, —(C═O)O—, —O(C═O)O—, —C(═O)—, —O—, —O(CR¹¹⁰R¹¹¹)ₛO—, —S—, —C(═O)S—, —SC(═O)—, —NR¹¹⁰C(═O)—, —C(═O)NR¹¹⁰—, —NR¹¹⁰C(═O)NR¹¹¹—, —NR¹¹⁰C(═S)—, —C(═S)NR¹¹⁰—, —NR¹¹⁰C(═S)NR¹¹¹—, —OC(═O)NR¹¹⁰—, —NR¹¹⁰C(═O)O—, —SC(═O)NR¹¹⁰— or —NR¹¹⁰C═O)S—.
R⁷, R⁸, and R⁹ are each independently H, substituted or unsubstituted C₁-C₃₀ alkyl, or substituted or unsubstituted 2- to 30-membered heteroalkyl.
Each R^{a31} and R^{a32} is independently H, substituted or unsubstituted C₁-C₁₂ alkyl, or substituted or unsubstituted 2- to 12-membered heteroalkyl.
Each R⁸⁰¹, R⁸⁰², R⁹⁰¹, R⁹⁰², R¹¹⁰, and R¹¹¹ is independently H, substituted or unsubstituted C₁-C₁₂ alkyl, or substituted or unsubstituted 2- to 12-membered heteroalkyl.
Each s is independently an integer from 1 to 4.

In an aspect, provided herein is a cationic lipid of formula (III)

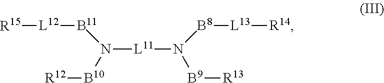

or a pharmaceutically acceptable salt, solvate, hydrate, stereoisomer, or prodrug thereof.
L¹¹ is

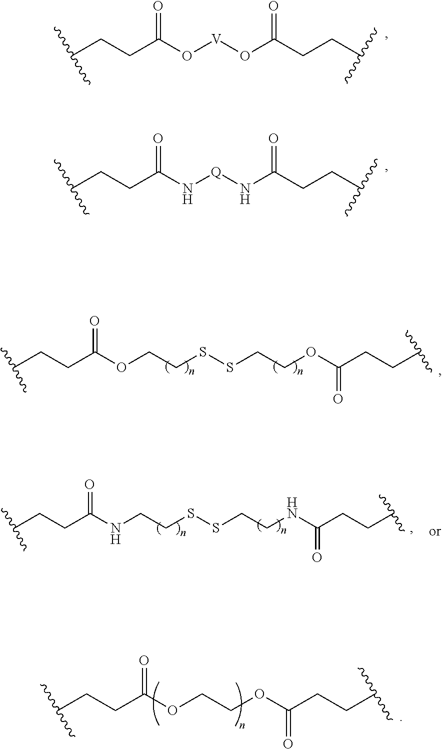

Q is substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene.
V is substituted or unsubstituted alkylene, substituted or unsubstituted cycloalkylene, substituted or unsubstituted arylene.
B⁸, B⁹, B¹⁰, and B¹¹ are each independently a bond, substituted or unsubstituted alkylene, or substituted or unsubstituted heteroalkylene.
L¹² is a bond, —O(C═O)—, —(C═O)O—, —O(C═O)O—, —C(═O)—, —O—, —O(CR²¹⁰R²¹¹)ₛO—, —S—, —C(═O)S—, —SC(═O)—, —NR²¹⁰C(═O)—, —C(═O)NR²¹⁰—, —NR²¹⁰C(═O)NR²¹¹—, —NR²¹⁰C(═S)—, —C(═S)NR²¹⁰, —NR²¹⁰C(═S)NR²¹¹—, —OC(═O)NR²¹⁰—, —NR²¹⁰C(═O)O—, —SC(═O)NR²¹⁰— or —NR²¹⁰C(═O)S—.
L¹³ is a bond, —O(C═O)—, —(C═O)O—, —O(C═O)O—, —C(═O)—, —O—, —O(CR³¹⁰R³¹¹)ₛO—, —S—, —C(═O)S—, —SC(═O)—, —NR³¹⁰C(═O)—, —C(═O)NR³¹⁰—, —NR³¹⁰C(═O)NR³¹¹—, —NR³¹⁰C(═S)—, —C(═S)NR³¹⁰—, —NR³¹⁰C(═S)NR³¹¹—, —OC(═O)NR³¹⁰—, —NR³¹⁰C(═O)O—, —SC(═O)NR³¹⁰— or —NR³¹⁰C(═O)S—.
R¹² is H, —OR^{12A}, —SR^{12A}, —NR^{12A}, —CN, —(C═O)R^{12A}, —O(C═O)R^{12A}, —(C═O)OR^{12A}, —NR^{12A}(C═O)—R^{12B}, —(C═O)NR^{12A}R^{12B}.
R¹³ is H, —OR^{13A}, —SR^{13A}, —NR^{13A}, —CN, —(C═O)R^{13A}, —O(C═O)R^{13A}, —(C═O)OR^{13A}, —NR^{13A}(C═O)—R^{13B}, —(C═O)NR^{13A}R^{13B}.
R¹⁴ and R¹⁵ are each independently substituted or unsubstituted C₂-C₃₀ alkyl, or substituted or unsubstituted 2- to 30-membered heteroalkyl.
R^{12A}, R^{12B}, R^{13A}, and R^{13B} are each independently H, substituted or unsubstituted C₁-C₂₀ alkyl, or substituted or unsubstituted 2 to 20 membered heteroalkyl.
Each R²¹⁰, R²¹¹, R³¹⁰, and R³¹¹ is independently H, substituted or unsubstituted C₁-C₁₂ alkyl, or substituted or unsubstituted 2- to 12-membered heteroalkyl.
Each n is independently an integer from 0 to 8, and
Each s is independently an integer from 1 to 4.

In an aspect, provided herein is a cationic lipid of formula (IV)

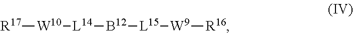

or a pharmaceutically acceptable salt, solvate, hydrate, stereoisomer, or prodrug thereof.
B¹² is —W⁷-L^{a3}-W⁸—.
W⁷ and W⁸ are each independently a bond, substituted or unsubstituted C₁-C₁₂ alkylene, or substituted or unsubstituted 2- to 12-membered heteroalkylene.
L^{a3} is a bond, —S—S—, —O—(CH₂O)ₘ—,

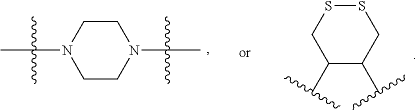 [SMILES: *N1CCN(*)CC1 ; *C1CSSCC1*]

W⁹ and W¹⁰ are each independently a bond, substituted or unsubstituted C₁-C₁₂ alkylene, substituted or unsubstituted 2- to 12-membered heteroalkylene, substituted or unsubstituted cycloalkylene, substituted or unsubstituted heterocycloalkylene, or any combination thereof.
L¹⁴ is a bond, —O(C═O)—, —(C═O)O—, —O(C═O)O—, —C(═O)—, —O—, —O(CR⁴¹⁰R⁴¹¹)ₛO—, —S—, —C(═O)S—, —SC(═O)—, —NR⁴¹⁰C(═O)—, —C(═O)NR⁴¹⁰—, —NR⁴¹⁰C(═O)NR⁴¹¹—, —NR⁴¹⁰C(═S)—, —C(═S)NR⁴¹⁰—, —NR⁴¹⁰C(═S)NR⁴¹—, —OC(═O)NR⁴¹⁰—, —NR⁴¹⁰C(═O)O—, —SC(═O)NR⁴¹⁰— or —NR⁴¹⁰C(═O)S—.
L¹⁵ is a bond, —O(C═O)—, —(C═O)O—, —O(C═O)O—, —C(═O)—, —O—, —O(CR⁵¹⁰R⁵¹¹)ₛO—, —S—, —C(═O)S—, —SC(═O)—, —NR⁵¹⁰C(═O)—, —C(═O)NR⁵¹⁰—, —NR⁵¹⁰C(═O)NR⁵¹¹—, —NR⁵¹⁰C(═S)—, —C(═S)NR⁵¹⁰—, —NR⁵¹⁰C(═S)NR⁵¹¹—, —OC(═O)NR⁵¹⁰—, —NR⁵¹⁰C(═O)O—, —SC(═O)NR⁵¹⁰— or —NR⁵¹⁰C(═O)S—.
R¹⁶ and R¹⁷ are each independently

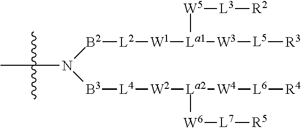

fragment of cationic lipid of formula (I),

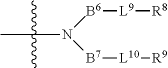

a fragment of cationic lipid of formula (II),

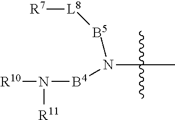

a fragment of cationic lipid of formula (II),

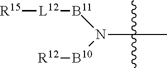

a fragment of cationic lipid of formula (III), or

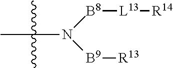

a fragment of cationic lipid of formula (III).
each R⁴¹⁰, R⁴¹¹, R⁵¹⁰, and R⁵¹¹ is independently H, substituted or unsubstituted C₁-C₁ alkyl, or substituted or unsubstituted 2- to 12-membered heteroalkyl.
each m is independently an integer from 0 to 8, and
each s is independently an integer from 1 to 4.

In an aspect, provided herein is a lipid nanoparticle comprising the cationic lipid of formulas I-IV as described herein.

In an aspect, provided herein is a pharmaceutical composition comprising a lipid nanoparticle which includes the cationic lipid of formulas I-IV as described herein, and a pharmaceutically acceptable carrier.

In an aspect, provided herein is a method for the in vivo delivery of a therapeutic agent, the method comprising administering the lipid nanoparticle which includes the cationic lipid of formulas I-IV as described herein.

In an aspect, provided herein is a method for treating a disease in a mammal in need thereof, the method comprising administering to the mammal a therapeutically effective amount of a lipid nanoparticle which includes the cationic lipid of formulas I-IV as described herein.

### BRIEF DESCRIPTION OF THE DRAWINGS

FIGS. 1A-D show the amount of anti-Spike antibody (of SARS-CoV-2) produced in mice, in response to injection of lipid nanoparticles comprising Spike mRNA. FIG. 1A shows the amount of anti-Spike antibody (of SARS-CoV-2) produced in mice, in response to injection of lipid nanoparticles comprising Spike mRNA Washington (WA) wild-type. FIG. 1B shows the amount of anti-Spike antibody (of SARS-CoV-2) produced in mice, in response to injection of lipid nanoparticles comprising Spike mRNA WA furin. FIG. 1C shows the amount of anti-Spike antibody (of SARS-CoV-2) produced in mice, in response to injection of lipid nanoparticles comprising Spike mRNA beta-furin. FIG. 1D shows the amount of anti-Spike antibody (of SARS-CoV-2) produced in mice, in response to injection of lipid nanoparticles comprising Spike mRNA beta-furin.

FIG. 2 shows the composition of the lipid nanoparticles used to generate the graphs shown in FIGS. 1A-D.

### DETAILED DESCRIPTION OF CERTAIN EMBODIMENTS

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, NY, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art. The features of any embodiment described herein can be combined with the features of any one or more other embodiments described herein, provided that the embodiments are not inconsistent with each other.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. In some embodiments, “about” encompasses variation within 10%, 5%, 2%, 1%, or 0.5% of a stated value.

Numeric ranges are inclusive of the numbers defining the range. Measured and measurable values are understood to be approximate, taking into account significant digits and the error associated with the measurement. Also, all ranges are to be interpreted as encompassing the endpoints in the absence of express exclusions such as “not including the endpoints”; thus, for example, “ranging from 1 to 10” includes the values 1 and 10 and all integer and (where appropriate) non-integer values greater than I and less than 10.

The term “coronavirus infection” refers to a human or animal that has cells that have been infected by a coronavirus. The infection can be established by performing a detection and/or viral titration from respiratory samples, or by assaying blood-circulating coronavirus-specific antibodies. The detection in the individuals infected with coronavirus is made by conventional diagnostic methods, such as molecular biology (e.g., PCR), which are known to those skilled in the art.

The use of “comprise”, “comprises”, “comprising”, “contain”, “contains”, “containing”, “include”, “includes”, and “including” and their grammatical variants, as used herein are intended to be non-limiting so that one item or multiple items in a list do not exclude other items that can be substituted or added to the listed items. It is to be understood that both the foregoing general description and detailed description are exemplary and explanatory only and are not restrictive of the teachings. Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The terms “effective amount”, “therapeutically effective amount” or “effective dose” or related terms may be used interchangeably and refer to an amount of the therapeutic agent that when administered to a subject, is sufficient to affect a measurable improvement or prevention of a disease or disorder associated with, for example, coronavirus infection. For example, administering an effective dose sufficient to inhibit the proliferation and/or replication of the coronavirus, and/or the development of the viral infection within the subject. Therapeutically effective amounts of the therapeutic agents provided herein, when used alone or in combination with another drug, will vary depending upon the relative activity of the therapeutic agent, and depending upon the subject and disease condition being treated, the weight and age and sex of the subject, the severity of the disease condition in the subject, the manner of administration and the like, which can readily be determined by one of ordinary skill in the art. In one embodiment, a therapeutically effective amount will depend on certain aspects of the subject to be treated and the disorder to be treated and may be ascertained by one skilled in the art using known techniques. In addition, as is known in the art, adjustments for age as well as the body weight, general health, sex, diet, time of administration, drug interaction, and the severity of the disease may be necessary.

The terms “subject” and “patient” as used herein refer to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The terms “administering”, “administered”, and grammatical variants thereof refer to the physical introduction of a therapeutic agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treating” is to be understood broadly and encompasses any beneficial effect, including, e.g., delaying, slowing, or arresting the worsening of symptoms associated with pulmonary inflammatory disease or remedying such symptoms, at least in part. Treating also encompasses bringing about any form of improved patient function, as discussed in detail below. In some embodiments, treatment also means prolonging survival as compared to expected survival if not receiving treatment. Those in need of treatment include those who already have the disease or disorder, as well as those who tend to have the disease or disorder or who should prevent the disease or disorder. In embodiments, the terms “treatment” and “treating” refer to fighting the coronavirus infection in a human or animal subject. By virtue of the administration of at least one embodiment of the compositions described herein, the viral infection rate (infectious titer) in the subject will decrease, and the virus may completely disappear from the subject. The terms “treatment” and “treating” also refers to attenuating symptoms associated with the viral infection (e.g., respiratory syndrome, kidney failure, fever, and other symptoms relating to coronavirus infections).

The term “synergistic effect” refers to a situation where the combination of two or more agents produces a greater effect than the sum of the effects of each of the individual agents. The term encompasses not only a reduction in symptoms of the disorder to be treated, but also an improved side effect profile, improved tolerability, improved patient compliance, improved efficacy, or any other improved clinical outcome.

The term a “sub-therapeutic amount” of an agent or therapy is an amount less than the effective amount for that agent or therapy as a single agent, but when combined with an effective or sub-therapeutic amount of another agent or therapy can produce a result desired by the physician, due to, for example, synergy in the resulting efficacious effects, or reduced side effects.

Combination therapy or “in combination with” refer to the use of more than one therapeutic agent to treat a particular disorder or condition. By “in combination with,” it is not intended to imply that the therapeutic agents must be administered at the same time and/or formulated for delivery together, although these methods of delivery are within the scope of this disclosure. A therapeutic agent can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. The therapeutic agents in a combination therapy can also be administered on an alternating dosing schedule, with or without a resting period (e.g., no therapeutic agent is administered on certain days of the schedule). The administration of a therapeutic agent “in combination with” another therapeutic agent includes, but is not limited to, sequential administration and concomitant administration of the two agents. In general, each therapeutic agent is administered at a dose and/or on a time schedule determined for that particular agent.

The terms “nucleic acid”, “polynucleotide” and “oligonucleotide” and other related terms used herein are used interchangeably and refer to polymers of nucleotides and are not limited to any particular length. The term refers to a polymer containing at least two deoxyribonucleotides or ribonucleotides in either single- or double-stranded form and includes DNA, RNA, and hybrids thereof. Nucleic acids include recombinant and chemically-synthesized forms. Nucleic acids include DNA molecules (cDNA or genomic DNA), RNA molecules (e.g., mRNA, siRNA, dsRNA, shRNA, miRNA, tRNA, rRNA, vRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In some embodiments, the nucleic acid molecules of the disclosure comprise a contiguous open reading frame encoding an Fc-coronavirus antigen fusion protein, or a derivative, mutein, or variant thereof. In one embodiment, nucleic acids comprise a one type of polynucleotides or a mixture of two or more different types of polynucleotides.

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules.

An “antibody” and “antibodies” and related terms used herein refers to an intact immunoglobulin or to an antigen binding portion thereof (or an antigen binding fragment thereof) that binds specifically to an antigen. Antigen binding portions (or the antigen binding fragment) may be produced by recombinant DNA techniques or by enzymatic or chemical cleavage of intact antibodies. Antigen binding portions (or antigen binding fragments) include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations.

A “neutralizing antibody” and related terms refers to an antibody that is capable of specifically binding to the neutralizing epitope of its target antigen (e.g., coronavirus spike protein) and substantially inhibiting or eliminating the biological activity of the target antigen (e.g., coronavirus spike protein). The neutralizing antibody can reduce the biological activity of the target antigen by at least 20%, 30%, 40%, 50%, 60%, 70%, 80%, 90% or higher levels of reduced biological activity.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes.

The terms “lipid” or “lipid moiety” are used in accordance with its ordinary meaning in chemistry and refer to a hydrophobic molecule which is typically characterized by an aliphatic hydrocarbon chain. In embodiments, the lipid moiety includes a carbon chain of 3 to 100 carbons. In embodiments, the lipid moiety includes a carbon chain of 5 to 50 carbons. In embodiments, the lipid moiety includes a carbon chain of 5 to 25 carbons. In embodiments, the lipid moiety includes a carbon chain of 8 to 525 carbons. Lipid moieties may include saturated or unsaturated carbon chains, and may be optionally substituted. In embodiments, the lipid moiety is optionally substituted with a charged moiety at the terminal end. In embodiments, the lipid moiety is an alkyl or heteroalkyl optionally substituted with a carboxylic acid moiety at the terminal end. Lipids are also a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The terms “cationic lipid” or “ionizable cationic lipid” are used interchangeably herein and refer to lipids that are protonated (e.g., >50% protonated) at low pH (e.g., pH 4), which makes them positively charged, but they may remain neutral at physiological pH (e.g., pH 7.4).

The term “lipid nanoparticle” includes a lipid formulation that can be used to deliver an active agent or therapeutic agent, such as a nucleic acid (e.g., an mRNA), to a target site of interest (e.g., cell, tissue, organ, and the like). In embodiments, the lipid particle described herein is a nucleic acid-lipid particle, e.g., formed from a cationic lipid, a non-cationic lipid, and optionally a conjugated lipid that prevents aggregation of the particle. In some embodiments, the active agent or therapeutic agent, such as a nucleic acid, may be encapsulated in the lipid portion of the particle, thereby protecting it from enzymatic degradation.

The term “lipid conjugate” refers to a conjugated lipid that inhibits aggregation of lipid particles. Such lipid conjugates include, but are not limited to, PEG-lipid conjugates such as, e.g., PEG coupled to dimyristoylglycerols (e.g., PEG-DMG conjugates), PEG coupled to diacylglycerols (e.g., PEG-DAG conjugates), PEG coupled to cholesterol, PEG coupled to phosphatidylethanolamines, and PEG conjugated to ceramides.

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C₁₂), myristoyl (C₁₄), palmitoyl (C₁₆), stearoyl (C₁₈), and icosoyl (C₂₀). In preferred embodiments, R¹ and R² are the same, i.e., R¹ and R² are both myristoyl (i.e., dimyristoyl), R and R² are both stearoyl (i.e., distearoyl), etc. Diacylglycerols have the following general formula:

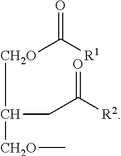

The term “dialkyloxypropyl” or “DAA” includes a compound having 2 alkyl chains, R¹ and R², both of which have independently between 2 and 30 carbons. The alkyl groups can be saturated or have varying degrees of unsaturation. Dialkyloxypropyls have the following general formula:

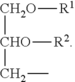

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The abbreviations used herein have their conventional meaning within the chemical and biological arts. The chemical structures and formulae set forth herein are constructed according to the standard rules of chemical valency known in the chemical arts. Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 30 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, B, Se, and S), and wherein the nitrogen and sulfur atoms may optionally be oxidized, and the nitrogen heteroatom may optionally be quaternized. The heteroatom(s) (e.g., O, N, S, Si, B, Se, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, B, Se, or P). A heteroalkyl moiety may include two optionally different heteroatoms (e.g., O, N, S, Si, B, Se, or P). A heteroalkyl moiety may include three optionally different heteroatoms (e.g., O, N, S, Si, B, Se, or P). A, heteroalkyl moiety may include four optionally different heteroatoms (e.g., O, N, S, Si, B, Se, or P). A heteroalkyl moiety may include five optionally different heteroatoms (e.g., O, N, S, Si, B, Se, or P). A heteroalkyl moiety may include up to 8 optionally different heteroatoms (e.g., O, N, S, Si, B, Se, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R¹¹ or the like, it will be understood that the terms heteroalkyl and —NR′R¹¹ are not redundant or mutually exclusive. Rather, the specific heteroalkyl groups are recited to add clarity. Thus, the term “heteroalkyl” should not be interpreted herein as excluding specific heteroalkyl groups, such as —NR′R¹¹ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5- or 6-membered monocyclic cycloalkyl ring fused to either a phenyl ring, a 5- or 6-membered monocyclic cycloalkyl, a 5- or 6-membered monocyclic cycloalkenyl, a 5- or 6-membered monocyclic heterocyclyl, or a 5- or 6-membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3-, 4-, 5-, 6- or 7-membered ring containing at least one heteroatom independently selected from the group consisting of O, N, and S where the ring is saturated or unsaturated, but not aromatic. The 3- or 4-membered ring contains 1 heteroatom selected from the group consisting of O, N and S. The 5-membered ring can contain zero or one double bond and one, two or three heteroatoms selected from the group consisting of O, N and S. The 6- or 7-membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5- or 6-membered monocyclic heterocyclyl ring fused to a phenyl ring, a 5- or 6-membered monocyclic cycloalkyl, a 5- or 6-membered monocyclic cycloalkenyl, a 5- or 6-membered monocyclic heterocyclyl, or a 5- or 6-membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

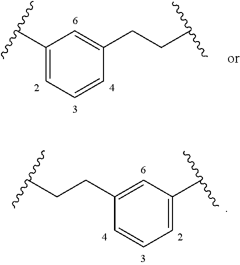 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2- to 5-membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R¹¹ are attached to the same nitrogen atom, they can be combined with the nitrogen atom to form a 4-, 5-, 6-, or 7-membered ring. For example, —NR′R¹¹ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR″, —S(O)R′, —S(O)₂R′, —S(O)₂NR′R″, —NRSO₂R′, —NR′NR″R′″, —ONR′R″, —NR′C(O)NR″NR′″R″″, —CN, —N₀₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂-, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —N₀₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₃₀ alkyl, C₁-C₈ alkyl, C₁-C₆ alkyl, or C₁-C₄ alkyl), unsubstituted heteroalkyl (e.g., 2- to 30-membered heteroalkyl, 2- to 8-membered heteroalkyl, 2- to 6-membered heteroalkyl, or 2- to 4-membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3- to 8-membered heterocycloalkyl, 3- to 6-membered heterocycloalkyl, or 5- to 6-membered heterocycloalkyl), unsubstituted aryl (e.g., C₆-C₁₀ aryl, C₁₀ aryl, or phenyl), or unsubstituted heteroaryl (e.g., 5- to 10-membered heteroaryl, 5- to 9-membered heteroaryl, or 5- to 6-membered heteroaryl), and
(B) alkyl (e.g., C₁-C₃₀ alkyl, C₁-C₈ alkyl, C₁-C₆ alkyl, or C₁-C₄ alkyl), heteroalkyl (e.g., 2- to 30-membered heteroalkyl, 2- to 8-membered heteroalkyl, 2- to 6-membered heteroalkyl, or 2- to 4-membered heteroalkyl), cycloalkyl (e.g., C₃-C₈ cycloalkyl, C₃-C₆ cycloalkyl, or C₅-C₆ cycloalkyl), heterocycloalkyl (e.g., 3- to 8-membered heterocycloalkyl, 3- to 6-membered heterocycloalkyl, or 5- to 6-membered heterocycloalkyl), aryl (e.g., C₆-C₁₀ aryl, C₁₀ aryl, or phenyl), heteroaryl (e.g., 5- to 10-membered heteroaryl, 5- to 9-membered heteroaryl, or 5- to 6-membered heteroaryl), substituted with at least one substituent selected from:
(i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —N₀₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₃₀ alkyl, C₁-C₈ alkyl, C₁-C₆ alkyl, or C₁-C₄ alkyl), unsubstituted heteroalkyl (e.g., 2- to 30-membered heteroalkyl, 2- to 8-membered heteroalkyl, 2- to 6-membered heteroalkyl, or 2- to 4-membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3- to 8-membered heterocycloalkyl, 3- to 6-membered heterocycloalkyl, or 5- to 6-membered heterocycloalkyl), unsubstituted aryl (e.g., C₆-C₁₀ aryl, C₁₀ aryl, or phenyl), or unsubstituted heteroaryl (e.g., 5- to 10-membered heteroaryl, 5- to 9-membered heteroaryl, or 5- to 6-membered heteroaryl), and
(ii) alkyl (e.g., C₁-C₃₀ alkyl, C₁-C₈ alkyl, C₁-C₆ alkyl, or C₁-C₄ alkyl), heteroalkyl (e.g., 2- to 30-membered heteroalkyl, 2- to 8-membered heteroalkyl, 2- to 6-membered heteroalkyl, or 2- to 4-membered heteroalkyl), cycloalkyl (e.g., C₃-C₈ cycloalkyl, C₃-C₆ cycloalkyl, or C₅-C₆ cycloalkyl), heterocycloalkyl (e.g., 3- to 8-membered heterocycloalkyl, 3- to 6-membered heterocycloalkyl, or 5- to 6-membered heterocycloalkyl), aryl (e.g., C₆-C₁₀ aryl, C₁₀ aryl, or phenyl), heteroaryl (e.g., 5- to 10-membered heteroaryl, 5- to 9-membered heteroaryl, or 5- to 6-membered heteroaryl), substituted with at least one substituent selected from:
(a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₃₀ alkyl, C₁-C₈ alkyl, C₁-C₆ alkyl, or C₁-C₄ alkyl), unsubstituted heteroalkyl (e.g., 2- to 30-membered heteroalkyl, 2- to 8-membered heteroalkyl, 2- to 6-membered heteroalkyl, or 2- to 4-membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3- to 8-membered heterocycloalkyl, 3- to 6-membered heterocycloalkyl, or 5- to 6-membered heterocycloalkyl), unsubstituted aryl (e.g., C₆-C₁₀ aryl, C₁₀ aryl, or phenyl), or unsubstituted heteroaryl (e.g., 5- to 10-membered heteroaryl, 5- to 9-membered heteroaryl, or 5- to 6-membered heteroaryl), and
(b) alkyl (e.g., C₁-C₃₀ alkyl, C₁-C₈ alkyl, C₁-C₆ alkyl, or C₁-C₄ alkyl), heteroalkyl (e.g., 2- to 30-membered heteroalkyl, 2- to 8-membered heteroalkyl, 2- to 6-membered heteroalkyl, or 2- to 4-membered heteroalkyl), cycloalkyl (e.g., C₃-C₈ cycloalkyl, C₃-C₆ cycloalkyl, or C₅-C₆ cycloalkyl), heterocycloalkyl (e.g., 3- to 8-membered heterocycloalkyl, 3- to 6-membered heterocycloalkyl, or 5- to 6-membered heterocycloalkyl), aryl (e.g., C₆-C₁₀ aryl, C₁₀ aryl, or phenyl), heteroaryl (e.g., 5- to 10-membered heteroaryl, 5- to 9-membered heteroaryl, or 5- to 6-membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₃₀ alkyl, C₁-C₈ alkyl, C₁-C₆ alkyl, or C₁-C₄ alkyl), unsubstituted heteroalkyl (e.g., 2- to 30-membered heteroalkyl, 2- to 8-membered heteroalkyl, 2- to 6-membered heteroalkyl, or 2- to 4-membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3- to 8-membered heterocycloalkyl, 3- to 6-membered heterocycloalkyl, or 5- to 6-membered heterocycloalkyl), unsubstituted aryl (e.g., C₆-C₁₀ aryl, C₁₀ aryl, or phenyl), or unsubstituted heteroaryl (e.g., 5- to 10-membered heteroaryl, 5- to 9-membered heteroaryl, or 5- to 6-membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₃₀ alkyl, each substituted or unsubstituted heteroalkyl is a substituted or unsubstituted 2- to 30-membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3- to 8-membered heterocycloalkyl, each substituted or unsubstituted aryl is a substituted or unsubstituted C₆-C₁₀ aryl, and each substituted or unsubstituted heteroaryl is a substituted or unsubstituted 5- to 10-membered heteroaryl.

A “lower substituent” or“lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2- to 8-membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3- to 7-membered heterocycloalkyl, each substituted or unsubstituted aryl is a substituted or unsubstituted phenyl, and each substituted or unsubstituted heteroaryl is a substituted or unsubstituted 5- to 6-membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₃₀ alkyl, each substituted or unsubstituted heteroalkyl is a substituted or unsubstituted 2- to 30-membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3- to 8-membered heterocycloalkyl, each substituted or unsubstituted aryl is a substituted or unsubstituted C₆-C₁₀ aryl, and/or each substituted or unsubstituted heteroaryl is a substituted or unsubstituted 5- to 10-membered heteroaryl. In some embodiments of the compounds herein, each substituted or unsubstituted alkylene is a substituted or unsubstituted C₁-C₃₀ alkylene, each substituted or unsubstituted heteroalkylene is a substituted or unsubstituted 2- to 30-membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3- to 8-membered heterocycloalkylene, each substituted or unsubstituted arylene is a substituted or unsubstituted C₆-C₁₀ arylene, and/or each substituted or unsubstituted heteroarylene is a substituted or unsubstituted 5- to 10-membered heteroarylene.

In some embodiments, each substituted or unsubstituted alkyl is a substituted or unsubstituted C₁-C₃₀ alkyl, each substituted or unsubstituted heteroalkyl is a substituted or unsubstituted 2- to 30-membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3- to 7-membered heterocycloalkyl, each substituted or unsubstituted aryl is a substituted or unsubstituted C₆-C₁₀ aryl, and/or each substituted or unsubstituted heteroaryl is a substituted or unsubstituted 5- to 9-membered heteroaryl. In some embodiments, each substituted or unsubstituted alkylene is a substituted or unsubstituted C₁-C₃₀ alkylene, each substituted or unsubstituted heteroalkylene is a substituted or unsubstituted 2- to 30-membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3- to 7-membered heterocycloalkylene, each substituted or unsubstituted arylene is a substituted or unsubstituted C₆-C₁₀ arylene, and/or each substituted or unsubstituted heteroarylene is a substituted or unsubstituted 5- to 9-membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)—, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

### Compounds

In an aspect, provided herein is cationic lipid of formula (I):

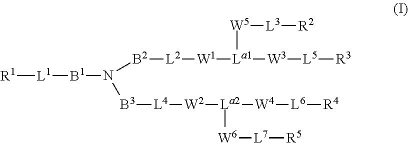

or a pharmaceutically acceptable salt, solvate, hydrate, stereoisomer, or prodrug thereof.
R¹ is H, —OR^{1A}, —YOR^{1A}, —NR^{1A}R^{1B}, —YNR^{1A}R^{1B}, —SR^{1A}, —YSR^{1A}, —(C═O)R^{1A}, —Y(C═O)R^{1A}, —(C═O)OR^{1A}, —Y(C═O)OR^{1A}, —O(C═O)R^{1A}, —YO(C═O)R^{1A}, —O(C═O)OR^{1A}, —YO(C═O)OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
Y is substituted or unsubstituted C₀-C₁₂ alkylene or substituted or unsubstituted 0- to 12-membered heteroalkylene.
R² is H, —OR^{2A}, —SR^{2A}, —(C═O)R^{2A}, —(C═O)OR^{2A}, —O(C═O)R^{2A}, —O(C═O)OR^{2A}, —(C═O)NHR^{2A}, —NH(C═O)R^{2A}, substituted or unsubstituted alkyl, or substituted or unsubstituted heteroalkyl.
R³ is H, —OR^{3A}, —SR^{3A}, —(C═O)R^{3A}, —(C═O)OR^{3A}, —O(C═O)R^{3A}, —O(C═O)OR^{3A}, —(C═O)NHR^{3A}, —NH(C═O)R^{3A}, substituted or unsubstituted alkyl, or substituted or unsubstituted heteroalkyl.
R⁴ is H, —OR^{4A}, —SR^{4A}, —(C═O)R^{4A}, —(C═O)OR^{4A}, —O(C═O)R^{4A}, —O(C═O)OR^{4A}, —(C═O)NHR^{4A}, —NH(C═O)R^{4A}, substituted or unsubstituted alkyl, or substituted or unsubstituted heteroalkyl.
R⁵ is H, —OR^{5A}, —SR^{5A}, —(C═O)R^{1A}, —(C═O)OR^{5A}, —O(C═O)R^{5A}, —O(C═O)OR^{5A}, —(C═O)NHR^{5A}, —NH(C═O)R^{5A}, substituted or unsubstituted alkyl, or substituted or unsubstituted heteroalkyl.
B¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
B² and B³ are each independently a bond, substituted or unsubstituted alkylene, or substituted or unsubstituted heteroalkylene.
L¹ is a bond, —O(C═O)—, —(C═O)O—, —O(C═O)O—, —C(═O)—, —O—, —O(CR¹⁰¹R¹⁰²)ₛO—, —S—, —C(═O)S—, —SC(═O)—, —NR¹⁰¹C(═O)—, —C(═O)NR¹⁰¹—, —NR¹⁰¹C(═S)—, —C(═S)NR¹⁰¹—, —NR¹⁰¹C(═O)NR¹⁰²—, —NR¹⁰¹C(═S)NR¹⁰²—, —OC(═O)NR¹⁰¹—, —NR¹⁰¹C(═O)O—, —SC(═O)NR¹⁰¹— or —NR¹⁰¹C(═O)S—.
L² is a bond, —O(C═O)—, —(C═O)O—, —O(C═O)O—, —C(═O)—, —O—, —O(CR²⁰¹R²⁰²)ₛO—, —S—, —C(═O)S—, —SC(═O)—, —NR²⁰¹C(═O)—, —C(═O)NR²⁰¹—, —NR²⁰¹C(═O)NR²⁰²—, —NR²⁰¹C(═S)—, —C(═S)NR²⁰¹—, —NR²⁰¹C(═S)NR²⁰²—, —OC(═O)NR²⁰¹—, —NR²⁰¹C(═O)O—, —SC(═O)NR²⁰¹— or —NR²⁰1C(═O)S—.
L³ is a bond, —O(C═O)—, —(C═O)O—, —O(C═O)O—, —C(═O)—, —O—, —O(CR³⁰¹R³⁰²)SO—, —S—, —C(═O)S—, —SC(═O)—, —NR³⁰¹C(═O)—, —C(═O)NR³⁰¹—, —NR³⁰¹C(═O)NR³⁰²—, —NR³⁰¹C(═S)—, —C(═S)NR³⁰¹—, —NR³⁰¹C(═S)NR³⁰²—, —OC(═O)NR³⁰¹—, —NR³⁰¹C(═O)O—, —SC(═O)NR³⁰¹— or —NR³⁰¹C(═O)S—.
L⁴ is a bond, —O(C═O)—, —(C═O)O—, —O(C═O)O—, —C(═O)—, —O—, —O(CR⁴⁰¹R⁴⁰²)ₛO—, —S—, —C(═O)S—, —SC(═O)—, —NR⁴⁰¹C(═O)—, —C(═O)NR⁴⁰¹—, —NR⁴⁰¹C(═O)NR⁴⁰²—, —NR⁴⁰¹C(═S)—, —C(═S)NR⁴⁰¹—, —NR⁴⁰¹C(═S)NR⁴⁰²—, —OC(═O)NR⁴⁰¹—, —NR⁴⁰¹C(═O)O—, —SC(═O)NR⁴⁰¹— or —NR⁴⁰¹C(═O)S—.
L⁵ is a bond, —O(C═O)—, —(C═O)O—, —O(C═O)O—, —C(═O)—, —O—, —O(CR⁵⁰¹R⁵⁰²)SO—, —S—, —C(═O)S—, —SC(═O)—, —NR⁵⁰¹C(═O)—, —C(═O)NR⁵⁰¹—, —NR⁵⁰¹C(═O)NR⁵⁰²—, —NR⁵⁰¹C(═S)—, —C(═S)NR⁵⁰¹—, —NR⁵⁰¹C(═S)NR⁵⁰²—, —OC(═O)NR⁵⁰¹—, —NR⁵⁰¹C(═O)O—, —SC(═O)NR⁵⁰¹— or —NR⁵⁰¹C(═O)S—.
L⁶ is a bond, —O(C═O)—, —(C═O)O—, —O(C═O)O—, —C(═O)—, —O—, —O(CR⁶⁰¹R⁶⁰²)ₛO—, —S—, —C(═O)S—, —SC(═O)—, —NR⁶⁰¹C(═O)—, —C(═O)NR⁶⁰¹—, —NR⁶⁰¹C(═O)NR⁶⁰²—, —NR⁶⁰¹C(═S)—, —C(═S)NR⁶⁰¹—, —NR⁶⁰¹C(═S)NR⁶⁰²—, —OC(═O)NR⁶⁰¹—, —NR⁶⁰¹C(═O)O—, —SC(═O)NR⁶⁰¹— or —NR⁶⁰¹C(═O)S—.
L⁷ is a bond, —O(C═O)—, —(C═O)O—, —O(C═O)O—, —C(═O)—, —O—, —O(CR⁷⁰¹R⁷⁰²)SO—, —S—, —C(═O)S—, —SC(═O)—, —NR⁷⁰¹C(═O)—, —C(═O)NR⁷⁰¹—, —NR⁷⁰¹C(═O)NR⁷⁰²—, —NR⁷¹C(═S)—, —C(═S)NR⁷⁰¹—, —NR⁷⁰¹C(═S)NR⁷⁰²—, —OC(═O)NR⁷⁰¹—, —NR⁷⁰¹C(═O)O—, —SC(═O)NR⁷⁰¹— or —NR⁷⁰¹C(═O)S—.
L^{a1} and L^{a2} are each independently

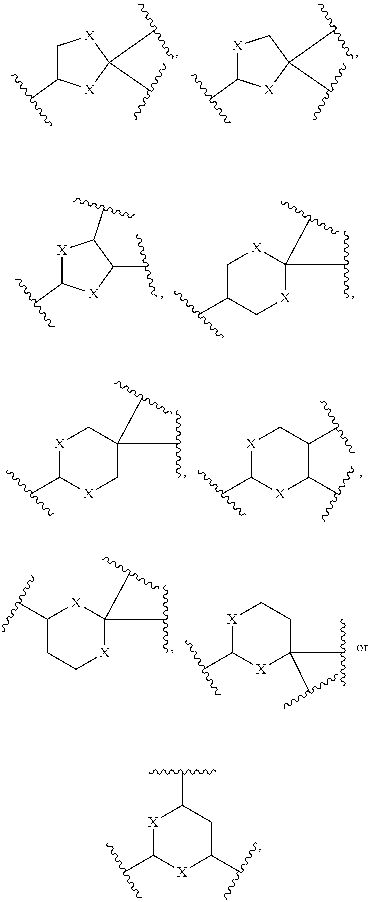

where each X is independently O, S, or CH₂.
W¹, W², W³, W⁴, W⁵, and W⁶ are each independently a bond, substituted or unsubstituted C₁-C₁₂ alkylene, or substituted or unsubstituted 2- to 12-membered heteroalkylene.
Each R^{1A} and R^{1B} is independently H, substituted or unsubstituted C₁-C₁₂ alkyl, or substituted or unsubstituted 2- to 12-membered heteroalkyl.
Each R^{2A}, R^{3A}, R^{4A}, and R^{5A} is independently H, substituted or unsubstituted C₁-C₃₀ alkyl, or substituted or unsubstituted 2- to 30-membered heteroalkyl.
Each R¹⁰¹, R¹⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently H, substituted or unsubstituted C₁-C₁₂ alkyl, or substituted or unsubstituted 2- to 12-membered heteroalkyl.
Each s is independently an integer from 1 to 4.

In embodiments, R¹ is independently H, —OR^{1A}, —YOR^{1A}, N^{1A}R^{1B}, —Y^{1A}R^{1B}, —SR^{1A}, —YSR^{1A}, —(C═O)R^{1A}, —Y(C═O)R^{1A}, —(C═O)OR^{1A}, —Y(C═O)OR^{1A}, —O(C═O)R^{1A}, —YO(C═O)R^{1A}, —O(C═O)OR^{1A}, —YO(C═O)OR^{1A}, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), substituted (e.g. with a substituent group, a size-limited substituent group or a lower substituent group) or unsubstituted heteroalkyl (e.g., 2- to 30-membered heteroalkyl, 2- to 8-membered heteroalkyl, or 2- to 4-membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3- to 8-membered heterocycloalkyl, 3- to 6-membered heterocycloalkyl, or 5- to 6-membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5- to 10-membered heteroaryl, 5- to 9-membered heteroaryl, or 5- to 6-membered heteroaryl). In embodiments, R¹ is substituted with one or more substituent groups. In embodiments, R¹ is substituted with one or more size-limited substituent groups. In embodiments, R¹ is substituted with one or more lower substituent groups.

In embodiments, R¹ is independently substituted (e.g. with a substituent group, a size-limited substituent group or a lower substituent group) alkyl (e.g., C₁-C₃₀ alkyl, C₁-C₈alkyl, or C₁-C₄ alkyl). In embodiments, R¹ is independently unsubstituted alkyl (e.g., C₁-C₃₀ alkyl, C₁-C₈ alkyl, or C₁-C₄ alkyl). In embodiments, R¹ is independently substituted (e.g. with a substituent group, a size-limited substituent group or a lower substituent group) heteroalkyl (e.g., 2- to 30-membered heteroalkyl, 2- to 8-membered heteroalkyl, or 2- to 4-membered heteroalkyl). In embodiments, R′ is independently unsubstituted heteroalkyl (e.g., 2- to 30-membered heteroalkyl, 2- to 8-membered heteroalkyl, or 2- to 4-membered heteroalkyl). In embodiments, R¹ is independently substituted (e.g. with a substituent group, a size-limited substituent group or a lower substituent group) cycloalkyl (e.g., C₃-C₈ cycloalkyl, C₃-C₆ cycloalkyl, or C₅-C₆ cycloalkyl). In embodiments, R¹ is independently unsubstituted cycloalkyl (e.g., C₃-C₈ cycloalkyl, C₃-C₆ cycloalkyl, or C₅-C₆ cycloalkyl). In embodiments, R¹ is independently substituted (e.g. with a substituent group, a size-limited substituent group or a lower substituent group) heterocycloalkyl (e.g., 3- to 8-membered heterocycloalkyl, 3- to 6-membered heterocycloalkyl, or 5- to 6-membered heterocycloalkyl). In embodiments, R¹ is independently unsubstituted heterocycloalkyl (e.g., 3- to 8-membered heterocycloalkyl, 3- to 6-membered heterocycloalkyl, or 5- to 6-membered heterocycloalkyl). In embodiments, R¹ is independently substituted (e.g. with a substituent group, a size-limited substituent group or a lower substituent group) aryl (e.g., C₆-C₁₀ aryl, C₁₀ aryl, or phenyl). In embodiments, R¹ is independently unsubstituted aryl (e.g., C₆-C₁₀ aryl, C₁₀ aryl, or phenyl). In embodiments, R¹ is independently substituted (e.g. with a substituent group, a size-limited substituent group or a lower substituent group) heteroaryl (e.g., 5- to 10-membered heteroaryl, 5- to 9-membered heteroaryl, or 5- to 6-membered heteroaryl). In embodiments, R¹ is independently unsubstituted heteroaryl (e.g., 5- to 10-membered heteroaryl, 5- to 9-membered heteroaryl, or 5- to 6-membered heteroaryl).

In embodiments, R¹ is independently H, —OR^{1A}, or substituted or unsubstituted heteroalkyl. In embodiments, R¹ is independently H, —OR^{1A}, or substituted (e.g. with a substituent group, a size-limited substituent group or a lower substituent group) or unsubstituted heteroalkyl (e.g., 2- to 30-membered heteroalkyl, 2- to 8-membered heteroalkyl, or 2- to 4-membered heteroalkyl). In embodiments, R¹ is independently H. In embodiments, R¹ is independently —OR^{1A}. In embodiments, R¹ is independently substituted or unsubstituted heteroalkyl. In embodiments, R′ is independently substituted (e.g. with a substituent group, a size-limited substituent group or a lower substituent group) or unsubstituted heteroalkyl (e.g., 2- to 30-membered heteroalkyl, 2- to 8-membered heteroalkyl, or 2- to 4-membered heteroalkyl).

In embodiments, R¹ is independently H, —OH, methoxy, ethoxy, or substituted or unsubstituted heteroalkyl. In embodiments, R′ is independently —OH or methoxy.

In embodiments, R¹ is independently H. In embodiments, R¹ is independently —OH. In embodiments, R′ is independently methoxy. In embodiments, R⁵ is independently ethoxy.

In embodiments, R² is H, —OR^{2A}, —SR^{2A}, —(C═O)R^{2A}, —(C═O)OR^{2A}, —O(C═O)R^{2A}, —O(C═O)OR^{2A}, —(C═O)NHR^{2A}, —NH(C═O)R^{2A}, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), or substituted (e.g. with a substituent group, a size-limited substituent group or a lower substituent group) or unsubstituted heteroalkyl (e.g., 2- to 30-membered heteroalkyl, 2- to 8-membered heteroalkyl, or 2- to 4-membered heteroalkyl). In embodiments, R² is substituted with one or more substituent groups. In embodiments, R² is substituted with one or more size-limited substituent groups. In embodiments, R² is substituted with one or more lower substituent groups.

In embodiments, R² is substituted (e.g. with a substituent group, a size-limited substituent group or a lower substituent group) alkyl (e.g., C₁-C₃₀ alkyl, C₁-C₈ alkyl, or C₁-C₄ alkyl). In embodiments, R² is unsubstituted alkyl (e.g., C₁-C₃₀ alkyl, C₁-C₈ alkyl, or C₁-C₄ alkyl). In embodiments, R² is substituted (e.g. with a substituent group, a size-limited substituent group or a lower substituent group) heteroalkyl (e.g., 2- to 30-membered heteroalkyl, 2- to 8-membered heteroalkyl, or 2- to 4-membered heteroalkyl). In embodiments, R² is unsubstituted heteroalkyl (e.g., 2- to 30-membered heteroalkyl, 2- to 8-membered heteroalkyl, or 2- to 4-membered heteroalkyl).

In embodiments, R² is H or substituted or unsubstituted alkyl. In embodiments, R² is H or substituted (e.g. with a substituent group, a size-limited substituent group or a lower substituent group) or unsubstituted alkyl (e.g., C₁-C₁₂ alkyl, C₁-C₈ alkyl, or C₁-C₄ alkyl). In embodiments, R² is H. In embodiments, R² is substituted (e.g. with a substituent group, a size-limited substituent group or a lower substituent group) alkyl (e.g., C₁-C₁₂ alkyl, C₁-C₈ alkyl, or C₁-C₄ alkyl). In embodiments, R² is unsubstituted alkyl (e.g., C₁-C₁₂ alkyl, C₁-C₈ alkyl, or CL-C₄ alkyl).

In embodiments, R² is H or substituted or unsubstituted C₁-C₁₂ alkyl. In embodiments, R² is substituted (e.g. with a substituent group, a size-limited substituent group or a lower substituent group) C₁-C₁₂ alkyl. In embodiments, R² is unsubstituted C₁-C₁₂ alkyl.

In embodiments, R³ is H, —OR^{3A}, —SR^{3A}, —(C═O)R^{3A}, —(C═O)OR^{3A}, —O(C═O)R^{3A}, —O(C═O)OR^{3A}, —(C═O)NHR^{3A}, —NH(C═O)R^{3A}, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), or substituted (e.g. with a substituent group, a size-limited substituent group or a lower substituent group) or unsubstituted heteroalkyl (e.g., 2- to 30-membered heteroalkyl, 2- to 8-membered heteroalkyl, or 2- to 4-membered heteroalkyl). In embodiments, R³ is substituted with one or more substituent groups. In embodiments, R³ is substituted with one or more size-limited substituent groups. In embodiments, R³ is substituted with one or more lower substituent groups.

In embodiments, R³ is substituted (e.g. with a substituent group, a size-limited substituent group or a lower substituent group) alkyl (e.g., C₁-C₃₀ alkyl, C₁-C₈ alkyl, or C₁-C₄ alkyl). In embodiments, R³ is unsubstituted alkyl (e.g., C₁-C₃₀ alkyl, C₁-C₈ alkyl, or C₁-C₄ alkyl). In embodiments, R³ is substituted (e.g. with a substituent group, a size-limited substituent group or a lower substituent group) heteroalkyl (e.g., 2- to 30-membered heteroalkyl, 2- to 8-membered heteroalkyl, or 2- to 4-membered heteroalkyl). In embodiments, R³ is unsubstituted heteroalkyl (e.g., 2- to 30-membered heteroalkyl, 2- to 8-membered heteroalkyl, or 2- to 4-membered heteroalkyl).

In embodiments, R³ is H or substituted or unsubstituted alkyl. In embodiments, R³ is H or substituted (e.g. with a substituent group, a size-limited substituent group or a lower substituent group) or unsubstituted alkyl (e.g., C₁-C₁₂ alkyl, C₁-C₈ alkyl, or C₁-C₄ alkyl). In embodiments, R³ is H. In embodiments, R³ is substituted (e.g. with a substituent group, a size-limited substituent group or a lower substituent group) alkyl (e.g., C₁-C₁₂ alkyl, C₁-C₈ alkyl, or C₁-C₄ alkyl). In embodiments, R³ is unsubstituted alkyl (e.g., C₁-C₁₂ alkyl, C₁-C₈ alkyl, or C₁-C₄ alkyl).

In embodiments, R³ is H or substituted or unsubstituted C₁-C₁₂ alkyl. In embodiments, R³ is substituted (e.g. with a substituent group, a size-limited substituent group or a lower substituent group) C₁-C₁₂ alkyl. In embodiments, R³ is unsubstituted C₁-C₁₂ alkyl.

In embodiments, R⁴ is H, —OR^{4A}, —SR^{4A}, —(C═O)R^{4A}, —(C═O)OR^{4A}, —O(C═O)R^{4A}, —O(C═O)OR^{4A}, —(C═O)NR^{4A}, —NH(C═O)R^{4A}, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), or substituted (e.g. with a substituent group, a size-limited substituent group or a lower substituent group) or unsubstituted heteroalkyl (e.g., 2- to 30-membered heteroalkyl, 2- to 8-membered heteroalkyl, or 2- to 4-membered heteroalkyl). In embodiments, R⁴ is substituted with one or more substituent groups. In embodiments, R⁴ is substituted with one or more size-limited substituent groups. In embodiments, R⁴ is substituted with one or more lower substituent groups.

In embodiments, R⁴ is substituted (e.g. with a substituent group, a size-limited substituent group or a lower substituent group) alkyl (e.g., C₁-C₃₀ alkyl, C₁-C₈ alkyl, or C₁-C₄ alkyl). In embodiments, R⁴ is unsubstituted alkyl (e.g., C₁-C₃₀ alkyl, C₁-C₈ alkyl, or C₁-C₄ alkyl). In embodiments, R⁴ is substituted (e.g. with a substituent group, a size-limited substituent group or a lower substituent group) heteroalkyl (e.g., 2- to 30-membered heteroalkyl, 2- to 8-membered heteroalkyl, or 2- to 4-membered heteroalkyl). In embodiments, R⁴ is unsubstituted heteroalkyl (e.g., 2- to 30-membered heteroalkyl, 2- to 8-membered heteroalkyl, or 2- to 4-membered heteroalkyl).

In embodiments, R⁴ is H or substituted or unsubstituted alkyl. In embodiments, R⁴ is H or substituted (e.g. with a substituent group, a size-limited substituent group or a lower substituent group) or unsubstituted alkyl (e.g., C₁-C₁₂ alkyl, C₁-C₈ alkyl, or C₁-C₄ alkyl). In embodiments, R⁴ is H. In embodiments, R⁴ is substituted (e.g. with a substituent group, a size-limited substituent group or a lower substituent group) alkyl (e.g., C₁-C₁₂ alkyl, C₁-C₈ alkyl, or C₁-C₄ alkyl). In embodiments, R⁴ is unsubstituted alkyl (e.g., C₁-C₁₂ alkyl, C₁-C₈ alkyl, or C₁-C₄ alkyl).

In embodiments, R⁴ is H or substituted or unsubstituted C₁-C₁₂ alkyl. In embodiments, R⁴ is substituted (e.g. with a substituent group, a size-limited substituent group or a lower substituent group) C₁-C₁₂ alkyl. In embodiments, R⁴ is unsubstituted C₁-C₁₂ alkyl.

In embodiments, R⁵ is H, —OR^{SA}, —SR^{5A}, —(C═O)R^{5A}, —(C═O)OR^{5A}, —O(C═O)R^{5A}, —O(C═O)OR^{5A}, —(C═O)NHR^{5A}, —NH(C═O)R^{5A}, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), or substituted (e.g. with a substituent group, a size-limited substituent group or a lower substituent group) or unsubstituted heteroalkyl (e.g., 2- to 30-membered heteroalkyl, 2- to 8-membered heteroalkyl, or 2- to 4-membered heteroalkyl). In embodiments, R⁵ is substituted with one or more substituent groups. In embodiments, R⁵ is substituted with one or more size-limited substituent groups. In embodiments, R⁵ is substituted with one or more lower substituent groups.

In embodiments, R⁵ is substituted (e.g. with a substituent group, a size-limited substituent group or a lower substituent group) alkyl (e.g., C₁-C₃₀ alkyl, C₁-C₈ alkyl, or C₁-C₄ alkyl). In embodiments, R⁵ is unsubstituted alkyl (e.g., C₁-C₃₀ alkyl, C₁-C₈ alkyl, or C₁-C₄ alkyl). In embodiments, R⁵ is substituted (e.g. with a substituent group, a size-limited substituent group or a lower substituent group) heteroalkyl (e.g., 2- to 30-membered heteroalkyl, 2- to 8-membered heteroalkyl, or 2- to 4-membered heteroalkyl). In embodiments, R⁵ is unsubstituted heteroalkyl (e.g., 2- to 30-membered heteroalkyl, 2- to 8-membered heteroalkyl, or 2- to 4-membered heteroalkyl).

In embodiments, R⁵ is H or substituted or unsubstituted alkyl. In embodiments, R⁵ is H or substituted (e.g. with a substituent group, a size-limited substituent group or a lower substituent group) or unsubstituted alkyl (e.g., C₁-C₁₂ alkyl, C₁-C₈ alkyl, or C₁-C₄ alkyl). In embodiments, R⁵ is H. In embodiments, R⁵ is substituted (e.g. with a substituent group, a size-limited substituent group or a lower substituent group) alkyl (e.g., C₁-C₁₂ alkyl, C₁-C₈ alkyl, or C₁-C₄ alkyl). In embodiments, R⁵ is unsubstituted alkyl (e.g., C₁-C₁₂ alkyl, C₁-C₈ alkyl, or C₁-C₄ alkyl).

In embodiments, R⁵ is H or substituted or unsubstituted C₁-C₁₂ alkyl. In embodiments, R′ is substituted (e.g. with a substituent group, a size-limited substituent group or a lower substituent group) C₁-C₁₂ alkyl. In embodiments, R⁵ is unsubstituted C₁-C₁₂ alkyl.

In embodiments, Y is substituted (e.g. with a substituent group, a size-limited substituent group or a lower substituent group) or unsubstituted C₀-C₁₂ alkylene or substituted (e.g. with a substituent group, a size-limited substituent group or a lower substituent group) or unsubstituted 0- to 12-membered heteroalkylene. In embodiments, Y is substituted with one or more substituent groups. In embodiments, Y is substituted with one or more size-limited substituent groups. In embodiments, Y is substituted with one or more lower substituent groups.

In embodiments, Y is substituted (e.g. with a substituent group, a size-limited substituent group or a lower substituent group) C₀-C₁₂ alkylene. In embodiments, Y is unsubstituted C₀-C₁₂ alkylene. In embodiments, Y is substituted (e.g. with a substituent group, a size-limited substituent group or a lower substituent group) 0- to 12-membered heteroalkylene. In embodiments, Y is unsubstituted 0- to 12-membered heteroalkylene.

In embodiments, Y is substituted (e.g. with a substituent group, a size-limited substituent group or a lower substituent group) C₁-C₈ alkylene. In embodiments, Y is unsubstituted C₁-C₈ alkylene. In embodiments, Y is substituted (e.g. with a substituent group, a size-limited substituent group or a lower substituent group) 1- to 8-membered heteroalkylene. In embodiments, Y is unsubstituted 1- to 8-membered heteroalkylene.

In embodiments, Y is substituted (e.g. with a substituent group, a size-limited substituent group or a lower substituent group) C₁-C₄ alkylene. In embodiments, Y is unsubstituted C₁-C₄ alkylene. In embodiments, Y is substituted (e.g. with a substituent group, a size-limited substituent group or a lower substituent group) 1- to 4-membered heteroalkylene. In embodiments, Y is unsubstituted 1- to 4-membered heteroalkylene.

In embodiments, Y is substituted (e.g. with a substituent group, a size-limited substituent group or a lower substituent group) methylene, ethylene or propylene. In embodiments, Y is unsubstituted methylene, ethylene or propylene.

In embodiments, B¹ is a bond, substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), substituted (e.g. with a substituent group, a size-limited substituent group or a lower substituent group) or unsubstituted heteroalkylene (e.g., 2- to 30-membered heteroalkylene, 2- to 8-membered heteroalkylene, or 2- to 4-membered heteroalkylene), substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene), substituted (e.g. with a substituent group, a size-limited substituent group or a lower substituent group) or unsubstituted heterocycloalkylene (e.g., 3- to 8-membered heterocycloalkylene, 3- to 6-membered heterocycloalkylene, or 5- to 6-membered heterocycloalkylene), substituted (e.g. with a substituent group, a size-limited substituent group or a lower substituent group) or unsubstituted arylene (e.g., C₆-C₁₀ arylene, C₁₀ arylene, or phenylene), or substituted (e.g. with a substituent group, a size-limited substituent group or a lower substituent group) or unsubstituted heteroarylene (e.g., 5- to 10-membered heteroarylene, 5- to 9-membered heteroarylene, or 5- to 6-membered heteroarylene). In embodiments, B¹ is substituted with one or more substituent groups. In embodiments, B¹ is substituted with one or more size-limited substituent groups. In embodiments, B¹ is substituted with one or more lower substituent groups. In embodiments, B¹ is a bond.

In embodiments, B¹ is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, B¹ is unsubstituted alkylene (e.g., C₁-C₃₀ alkylene, C₁-C₈ alkylene, or C₁-C₄ alkylene). In embodiments, B¹ is substituted (e.g. with a substituent group, a size-limited substituent group or a lower substituent group) heteroalkylene (e.g., 2- to 30-membered heteroalkylene, 2- to 8-membered heteroalkylene, or 2- to 4-membered heteroalkylene). In embodiments, B¹ is unsubstituted heteroalkylene (e.g., 2- to 30-membered heteroalkylene, 2- to 8-membered heteroalkylene, or 2- to 4-membered heteroalkylene). In embodiments, B¹ is substituted (e.g. with a substituent group, a size-limited substituent group or a lower substituent group) cycloalkylene (e.g., C₃-C₈ cycloalkylene, C₃-C₆ cycloalkylene, or C₅-C₆ cycloalkylene). In embodiments, B¹ is unsubstituted cycloalkylene (e.g., C₃-C₈ cycloalkylene, C₃-C₆ cycloalkylene, or C₅-C₆ cycloalkylene). In embodiments, B¹ is substituted (e.g. with a substituent group, a size-limited substituent group or a lower substituent group) heterocycloalkylene (e.g., 3- to 8-membered heterocycloalkylene, 3- to 6-membered heterocycloalkylene, or 5- to 6-membered heterocycloalkylene). In embodiments, B¹ is unsubstituted heterocycloalkylene (e.g., 3- to 8-membered heterocycloalkylene, 3- to 6-membered heterocycloalkylene, or 5- to 6-membered heterocycloalkylene). In embodiments, B¹ is substituted (e.g. with a substituent group, a size-limited substituent group or a lower substituent group) arylene (e.g., C₆-C₁₀ arylene, C₁₀ arylene, or phenylene). In embodiments, B¹ is unsubstituted arylene (e.g., C₆-C₁₀ arylene, C₁₀ arylene, or phenylene). In embodiments, B¹ is substituted (e.g. with a substituent group, a size-limited substituent group or a lower substituent group) heteroarylene (e.g., 5- to 10-membered heteroarylene, 5- to 9-membered heteroarylene, or 5- to 6-membered heteroarylene). In embodiments, B¹ is unsubstituted heteroarylene (e.g., 5- to 10-membered heteroarylene, 5- to 9-membered heteroarylene, or 5- to 6-membered heteroarylene).

In embodiments, B¹ is a bond or a substituted or unsubstituted alkylene. In embodiments, B¹ is a bond or substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene).

In embodiments, B¹ is a bond or unsubstituted alkylene. In embodiments, B¹ is a bond or unsubstituted C₁-C₈ alkylene. In embodiments, B¹ is unsubstituted alkylene. In embodiments, B¹ is unsubstituted C₁-C₈ alkylene. In embodiments, B¹ is a bond.

In embodiments, B² and B³ are each independently a bond, substituted or unsubstituted alkylene, or substituted or unsubstituted heteroalkylene. In embodiments, B² and B³ are each independently a bond, substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), or substituted (e.g. with a substituent group, a size-limited substituent group or a lower substituent group) or unsubstituted heteroalkylene (e.g., 2- to 30-membered heteroalkylene, 2- to 8-membered heteroalkylene, or 2- to 4-membered heteroalkylene). In embodiments, B² is substituted with one or more substituent groups. In embodiments, B² is substituted with one or more size-limited substituent groups. In embodiments, B 2 is substituted with one or more lower substituent groups. In embodiments, B² is a bond. In embodiments, B³ is substituted with one or more substituent groups. In embodiments, B 3 is substituted with one or more size-limited substituent groups. In embodiments, B 3 is substituted with one or more lower substituent groups. In embodiments, B³ is a bond.

In embodiments, B² is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, B² is unsubstituted alkylene (e.g., C₁-C₃₀ alkylene, C₁-C₈ alkylene, or C₁-C₄ alkylene). In embodiments, B² is substituted (e.g. with a substituent group, a size-limited substituent group or a lower substituent group) heteroalkylene (e.g., 2- to 30-membered heteroalkylene, 2- to 8-membered heteroalkylene, or 2- to 4-membered heteroalkylene). In embodiments, B² is unsubstituted heteroalkylene (e.g., 2- to 30-membered heteroalkylene, 2- to 8-membered heteroalkylene, or 2- to 4-membered heteroalkylene).

In embodiments, B³ is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, B³ is unsubstituted alkylene (e.g., C₁-C₃₀ alkylene, C₁-C₈ alkylene, or C₁-C₄ alkylene). In embodiments, B³ is substituted (e.g. with a substituent group, a size-limited substituent group or a lower substituent group) heteroalkylene (e.g., 2- to 30-membered heteroalkylene, 2- to 8-membered heteroalkylene, or 2- to 4-membered heteroalkylene). In embodiments, B³ is unsubstituted heteroalkylene (e.g., 2- to 30-membered heteroalkylene, 2- to 8-membered heteroalkylene, or 2- to 4-membered heteroalkylene).

In embodiments, B² and B³ are each independently a bond or substituted or unsubstituted alkylene. In embodiments, B² and B³ are each independently a bond or substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene).

In embodiments, B² and B³ are each independently a bond or substituted or unsubstituted C₁-C₈ alkylene. In embodiments, B² and B³ are each independently a bond or substituted (e.g. with a substituent group, a size-limited substituent group or a lower substituent group) or unsubstituted C₁-C₈ alkylene.

In embodiments, B² is a bond. In embodiments, B² is substituted C₁-C₈ alkylene. In embodiments, B² is unsubstituted C₁-C₈ alkylene. In embodiments, B³ is a bond. In embodiments, B³ is substituted C₁-C₈ alkylene. In embodiments, B³ is unsubstituted C₁-C₈ alkylene.

In embodiments, B² is butylene. In embodiments, B² is propylene. In embodiments, B² is ethylene. In embodiments, B² is methylene. In embodiments, B³ is butylene. In embodiments, B³ is propylene. In embodiments, B³ is ethylene. In embodiments, B³ is methylene.

In embodiments, L² is a bond, —O(C═O)—, —(C═O)O—, —O(C═O)O—, —C(═O)—, —O—, or —S—. In embodiments, L² is a bond, —O(C═O)—, —(C═O)O—, or —C(═O)—. In embodiments, L² is a bond, —O(C═O)— or —(C═O)O—.

In embodiments, L¹ is a bond, —NR¹⁰¹C(═S)—, —C(═S)NR¹⁰¹—, —O(C═O)—, —(C═O)O—, or —O—. In embodiments, L¹ is a bond, —NR¹⁰¹C(═S)—, or —C(═S)NR¹⁰¹—.

In embodiments, L¹ is a bond. In embodiments, L¹ is —NR¹⁰¹C(═S)—. In embodiments, L¹ is —C(═S)NR¹⁰¹. In embodiments, L¹ is —O(C═O)—. In embodiments, L¹ is —(C═O)O—. In embodiments, L¹ is —O—. In embodiments, L¹ is —C(═S)NR¹⁰¹, where the carbon atom is connected to the nitrogen atom in formula (I). In embodiments, L¹ is —C(═S)NH, where the carbon atom is connected to the nitrogen atom in formula (I).

In embodiments, each R¹⁰¹ is independently H, substituted or unsubstituted C₁-C₁₂ alkyl, or substituted or unsubstituted 2- to 12-membered heteroalkyl. In embodiments, each R¹⁰¹ is independently H, substituted (e.g. with a substituent group, a size-limited substituent group or a lower substituent group) or unsubstituted C₁-C₁₂ alkyl, or substituted or unsubstituted 2- to 12-membered heteroalkyl. In embodiments, each R¹⁰¹ is substituted with one or more substituent groups. In embodiments, each R¹⁰¹ is substituted with one or more size-limited substituent groups. In embodiments, each R¹⁰¹ is substituted with one or more lower substituent groups.

In embodiments, each R¹⁰¹ is independently H or substituted (e.g. with a substituent group, a size-limited substituent group or a lower substituent group) or unsubstituted 2- to 12-membered heteroalkyl. In embodiments, each R¹⁰¹ is independently substituted (e.g. with a substituent group, a size-limited substituent group or a lower substituent group) or unsubstituted 2- to 12-membered heteroalkyl.

In embodiments, each R¹⁰¹ is independently H. In embodiments, each R¹⁰¹ is independently substituted (e.g. with a substituent group, a size-limited substituent group or a lower substituent group) 2- to 12-membered heteroalkyl. In embodiments, each R¹⁰¹ is independently unsubstituted 2- to 12-membered heteroalkyl.

In embodiments, L² is a bond. In embodiments, L² is —O(C═O)—. In embodiments, L² is —(C═O)O—. In embodiments, L² is —C(═O)—. In embodiments, L² is —O(C═O)O—. In embodiments, L² is —S—. In embodiments, L² is —O—.

In embodiments, L³ is a bond, —O(C═O)—, —(C═O)O—, —O(C═O)O—, —C(═O)—, —O—, or —S—. In embodiments, L³ is a bond, —O(C═O)—, —(C═O)O—, or —C(═O)—. In embodiments, L³ is a bond, —O(C═O)— or —(C═O)O—.

In embodiments, L³ is a bond. In embodiments, L³ is —O(C═O)—. In embodiments, L³ is —(C═O)O—. In embodiments, L³ is —C(═O)—. In embodiments, L³ is —O(C═O)O—. In embodiments, L³ is —S—. In embodiments, L³ is —O—.

In embodiments, L⁴ is a bond, —O(C═O)—, —(C═O)O—, —O(C═O)O—, —C(═O)—, —O—, or —S—. In embodiments, L⁴ is a bond, —O(C═O)—, —(C═O)O—, or —C(═O)—. In embodiments, L⁴ is a bond, —O(C═O)— or —(C═O)O—.

In embodiments, L⁴ is a bond. In embodiments, L⁴ is —O(C═O)—. In embodiments, L⁴ is —(C═O)O—. In embodiments, L⁴ is —C(═O)—. In embodiments, L⁴ is —O(C═O)O—. In embodiments, L⁴ is —S—. In embodiments, L⁴ is —O—.

In embodiments, L⁵ is a bond, —O(C═O)—, —(C═O)O—, —O(C═O)O—, —C(═O)—, —O—, or —S—. In embodiments, L^{S} is a bond, —O(C═O)—, —(C═O)O—, or —C(═O)—. In embodiments, L⁵ is a bond, —O(C═O)— or —(C═O)O—.

In embodiments, L⁵ is a bond. In embodiments, L^{S} is —O(C═O)—. In embodiments, L⁵ is —(C═O)O—. In embodiments, L⁵ is —C(═O)—. In embodiments, L⁵ is —O(C═O)O—. In embodiments, L⁵ is —S—. In embodiments, L⁵ is —O—.

In embodiments, L⁶ is a bond, —O(C═O)—, —(C═O)O—, —O(C═O)O—, —C(═O)—, —O—, or —S—. In embodiments, L⁶ is a bond, —O(C═O)—, —(C═O)O—, or —C(═O)—. In embodiments, L⁶ is a bond, —O(C═O)— or —(C═O)O—.

In embodiments, L⁶ is a bond. In embodiments, L⁶ is —O(C═O)—. In embodiments, L⁶ is —(C═O)O—. In embodiments, L⁶ is —C(═O)—. In embodiments, L⁶ is —O(C═O)O—. In embodiments, L⁶ is —S—. In embodiments, L⁶ is —O—.

In embodiments, L⁷ is a bond, —O(C═O)—, —(C═O)O—, —O(C═O)O—, —C(═O)—, —O—, or —S—. In embodiments, L⁷ is a bond, —O(C═O)—, —(C═O)O—, or —C(═O)—. In embodiments, L⁷ is a bond, —O(C═O)— or —(C═O)O—.

In embodiments, L⁷ is a bond. In embodiments, L⁷ is —O(C═O)—. In embodiments, L⁷ is —(C═O)O—. In embodiments, L⁷ is —C(═O)—. In embodiments, L⁷ is —O(C═O)O—. In embodiments, L⁷ is —S—. In embodiments, L⁷ is —O—.

In embodiments, L^{a1} and L^{a2} are each independently

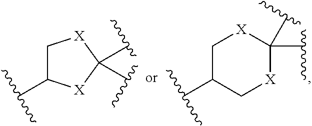

where each X is independently O or S. In embodiments, L^{a1} and L^{a2} are each independently

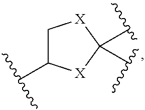

where each X is independently O or S. In embodiments, L^{a1} and L^{a2} are each independently

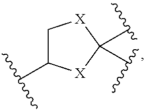

where each X is independently O.

In embodiments, L^{a1} and L^{a2} are each independently

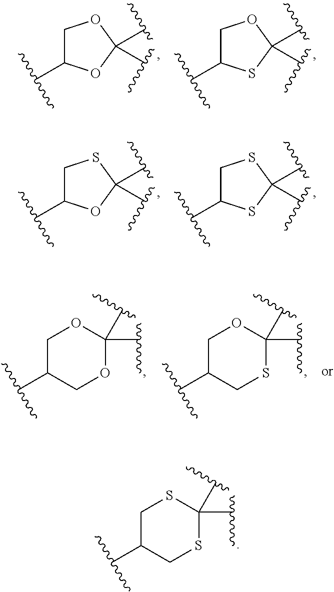

In embodiments, L^{a1} and L^{a2} are each independently

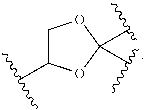 [SMILES: *C1COC(*)(*)O1]

In embodiments, L^{a1} and L^{a2} are each independently

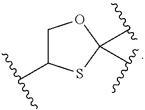 [SMILES: *C1COC(*)(*)S1]

In embodiments, L^{a1} and L^{a2} are each independently

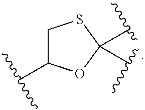 [SMILES: *C1CSC(*)(*)O1]

In embodiments, L^{a1} and L^{a2} are each independently

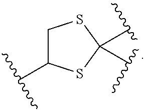 [SMILES: *C1CSC(*)(*)S1]

In embodiments, L^{a1} and L^{a2} are each independently

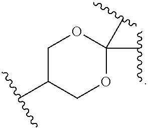 [SMILES: *C1COC(*)(*)OC1]

In embodiments, L^{a1} and L^{a2} are each independently

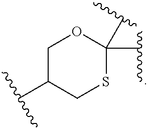 [SMILES: *C1COC(*)(*)SC1]

In embodiments, L^{a1} and L^{a2} are each independently

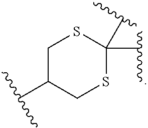 [SMILES: *C1CSC(*)(*)SC1]

In embodiments, W¹, W², W³, W⁴, W⁵, and W⁶ are each independently a bond, substituted (e.g. with a substituent group, a size-limited substituent group or a lower substituent group) or unsubstituted C₁-C₁₂ alkylene, or substituted (e.g. with a substituent group, a size-limited substituent group or a lower substituent group) or unsubstituted 2- to 12-membered heteroalkylene. In embodiments, W¹, W², W³, W⁴, W⁵, and W⁶ are each independently substituted with one or more substituent groups. In embodiments, W¹, W², W³, W⁴, W⁵, and W⁶ are each independently substituted with one or more size-limited substituent groups. In embodiments, W¹, W², W³, W⁴, W⁵, and W⁶ are each independently substituted with one or more lower substituent groups.

In embodiments, W¹, W², W³, W⁴, W⁵, and W⁶ are each independently a bond or substituted (e.g. with a substituent group, a size-limited substituent group or a lower substituent group) or unsubstituted C₁-C₁₂ alkylene. In embodiments, W¹, W², W³, W⁴, W⁵, and W⁶ are each independently a bond. In embodiments, W¹, W², W³, W⁴, W⁵, and W⁶ are each independently substituted (e.g. with a substituent group, a size-limited substituent group or a lower substituent group) C₁-C₁₂ alkylene. In embodiments, W¹, W², W³, W⁴, W⁵, and W⁶ are each independently unsubstituted C₁-C₁₂ alkylene.

In embodiments, each R^{1A} and R^{1B} is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2- to 12-membered heteroalkyl. In embodiments, each R^{1A} and R^{1B} is independently substituted with is independently substituted with one or more substituent groups. In embodiments, each R^{1A} and R^{1B} is independently substituted with one or more size-limited substituent groups. In embodiments, each R^{1A} and R^{1B} is independently substituted with one or more lower substituent groups.

In embodiments, R^{1A} and R^{1B} substituents bonded to the same nitrogen atom may optionally be joined to form a substituted or unsubstituted heterocycloalkyl (e.g., 3- to 8-membered, 3- to 6-membered, 4- to 6-membered, 4- to 5-membered, or 5- to 6-membered) or substituted or unsubstituted heteroaryl (e.g., 5- to 12-membered, 5- to 10-membered, 5- to 9-membered, or 5- to 6-membered). In embodiments, a substituted heterocycloalkyl or substituted heteroaryl formed by the joining of R^{1A} and R^{1B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{1A} and R^{1B} substituents bonded to the same nitrogen atom is substituted, it is substituted with at least one substituent group. In embodiments, when a heterocycloalkyl formed by the joining of R^{1A} and R^{1B} substituents bonded to the same nitrogen atom is substituted, it is substituted with at least one size-limited substituent group. In embodiments, when a heterocycloalkyl formed by the joining of R^{1A} and R^{1B} substituents bonded to the same nitrogen atom is substituted, it is substituted with at least one lower substituent group. In embodiments, when a heteroaryl formed by the joining of R^{1A} and R^{1B} substituents bonded to the same nitrogen atom is substituted, it is substituted with at least one substituent group. In embodiments, when a heteroaryl formed by the joining of R^{1A} and R^{1B} substituents bonded to the same nitrogen atom is substituted, it is substituted with at least one size-limited substituent group. In embodiments, when a heteroaryl formed by the joining of R^{1A} and R^{1B} substituents bonded to the same nitrogen atom is substituted, it is substituted with at least one lower substituent group.

In embodiments, each R^{1A} is independently H or substituted or unsubstituted C₁-C₁₂ alkyl. In embodiments, each R^{1A} is independently H or substituted (e.g. with a substituent group, a size-limited substituent group or a lower substituent group) or unsubstituted C₁-C₁₂ alkyl. In embodiments, each R^{1A} is independently H. In embodiments, each R^{1A} is independently substituted (e.g. with a substituent group, a size-limited substituent group or a lower substituent group) C₁-C₁₂ alkyl. In embodiments, each R^{1A} is independently unsubstituted C₁-C₁₂ alkyl.

In embodiments, R¹ is H, —ORA or substituted or unsubstituted heteroalkyl.
L¹ is a bond, —NR¹C(═S)—, —C(═S)NR¹⁰¹—, —O(C═O)—, —(C═O)O—, or —O—.
B¹ is a bond or a substituted or unsubstituted alkylene.
B² and B³ are each independently a bond or substituted or unsubstituted alkylene.
L² is a bond, —O(C═O)—, —(C═O)O—, —O(C═O)O—, —C(═O)—, —O—, or —S—.
L⁴ is a bond, —O(C═O)—, —(C═O)O—, —O(C═O)O—, —C(═O)—, —O—, or —S—.
W¹, W², W³, W⁴, W⁵, and W⁶ are each independently a bond or substituted or unsubstituted C₁-C₁₂ alkylene.
L^{a1} and L^{a2} are each independently

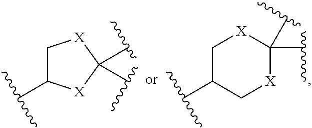

where each X is independently O or S.
L³ is a bond, —O(C═O)—, —(C═O)O—, —O(C═O)O—, —C(═O)—, —O—, or —S—.
L⁵ is a bond, —O(C═O)—, —(C═O)O—, —O(C═O)O—, —C(═O)—, —O—, or —S—.
L^{e} is a bond, —O(C═O)—, —(C═O)O—, —O(C═O)O—, —C(═O)—, —O—, or —S—.
L⁷ is a bond, —O(C═O)—, —(C═O)O—, —O(C═O)O—, —C(═O)—, —O—, or —S—.
R² is H or substituted or unsubstituted alkyl.
R³ is H or substituted or unsubstituted alkyl.
R⁴ is H or substituted or unsubstituted alkyl.
R⁵ is H or substituted or unsubstituted alkyl.
each R^{1A} is independently H or substituted or unsubstituted C₁-C₁₂ alkyl, and
each R¹⁰¹ is independently H or substituted or unsubstituted 2- to 12-membered heteroalkyl.

In embodiments, R¹ is H, —OH, methoxy, ethoxy, or substituted or unsubstituted heteroalkyl.
L¹ is a bond, —NR¹⁰¹C(═S)—, or —C(═S)NR¹⁰¹—.
B¹ is a bond or an unsubstituted C₁-C₈ alkylene.
B² and B³ are each independently a bond or substituted or unsubstituted C₁-C₈ alkylene.
L² is a bond, —O(C═O)—, or —(C═O)O—.
L⁴ is a bond, —O(C═O)—, or —(C═O)O—.
W¹, W², W³, W⁴, W⁵, and W⁶ are each independently a bond or substituted or unsubstituted C₁-C₁₂ alkylene.

L^{a1} and L^{a2} are each independently

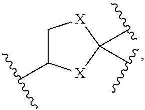

where each X is independently O or S.
L³ is a bond, —O(C═O)—, or —(C═O)O—.
L⁵ is a bond, —O(C═O)—, or —(C═O)O—.
L⁶ is a bond, —O(C═O)—, or —(C═O)O—.
L⁷ is a bond, —O(C═O)—, or —(C═O)O—.
R² is H or substituted or unsubstituted C₁-C₁₂ alkyl.
R³ is H or substituted or unsubstituted C₁-C₁₂ alkyl.
R⁴ is H or substituted or unsubstituted C₁-C₁₂ alkyl.
R⁵ is H or substituted or unsubstituted C₁-C₁₂ alkyl, and
each R¹⁰¹ is independently substituted or unsubstituted 2- to 12-membered heteroalkyl.

In embodiments, R¹ is —OH or methoxy. L¹ is a bond.
B¹ is an unsubstituted C₁-C₈ alkylene.
B² and B³ are each independently a bond or substituted or unsubstituted C₁-C₈ alkylene;
L² is a bond. L⁴ is a bond.
W¹, W², W³, W⁴, W⁵, and W⁶ are each independently a bond or substituted or unsubstituted C₁-C₁₂ alkylene.
L^{a1} and L^{a2} are each independently

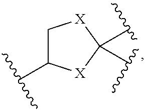

where each X is independently O.
L³ is a bond. L⁵ is a bond. L⁶ is a bond. L⁷ is a bond. R² is H or substituted or unsubstituted C₁-C₁₂ alkyl. R³ is H or substituted or unsubstituted C₁-C₁₂ alkyl.
R⁴ is H or substituted or unsubstituted C₁-C₁₂ alkyl, and R⁵ is H or substituted or unsubstituted C₁-C₁₂ alkyl;

In embodiments, R¹ is substituted or unsubstituted heteroalkyl.
L¹ is —C(═S)NR¹⁰¹—, where the carbon atom is connected to the nitrogen atom in formula (I).
B¹ is a bond. B² and B³ are each independently a bond or substituted or unsubstituted C₁-C₈ alkylene. L² is a bond, —O(C═O)—, or —(C═O)O—. L⁴ is a bond, —O(C═O)—, or —(C═O)O—.
W¹, W², W³, W⁴, W⁵, and W⁶ are each independently a bond or substituted or unsubstituted C₁-C₁₂ alkylene.
L^{a1} and L^{a2} are each independently

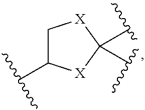

where each X is independently O. L³ is a bond.
L⁵ is a bond. L⁶ is a bond. L⁷ is a bond. R² is H or substituted or unsubstituted C₁-C₁₂ alkyl.
R³ is H or substituted or unsubstituted C₁-C₁₂ alkyl. R⁴ is H or substituted or unsubstituted C₁-C₁₂ alkyl, and R⁵ is H or substituted or unsubstituted C₁-C₁₂ alkyl.

In embodiments, each R^{2A}, R^{3A}, R^{4A}, and R^{5A} is independently H, substituted (e.g. with a substituent group, a size-limited substituent group or a lower substituent group) or unsubstituted C₁-C₃₀ alkyl, or substituted (e.g. with a substituent group, a size-limited substituent group or a lower substituent group) or unsubstituted 2- to 30-membered heteroalkyl. In embodiments, each R^{2A}, R^{3A}, R^{4A}, and R^{5A} is independently H. In embodiments, each R^{2A}, R^{3A}, R^{4A}, and R^{5A} is independently substituted (e.g. with a substituent group, a size-limited substituent group or a lower substituent group) C₁-C₃₀ alkyl. In embodiments, each R^{2A}, R^{3A}, R^{4A}, and R^{5A} is independently unsubstituted C₁-C₃₀ alkyl. In embodiments, each R^{2A}, R^{3A}, R^{4A}, and R^{5A} is independently substituted (e.g. with a substituent group, a size-limited substituent group or a lower substituent group) 2- to 30-membered heteroalkyl. In embodiments, each R^{2A}, R^{3A}, R^{4A} and R^{5A} is independently unsubstituted 2- to 30-membered heteroalkyl.

In embodiments, each R¹⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰, and R⁷⁰² is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2- to 12-membered heteroalkyl. In embodiments, each R¹⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently H. In embodiments, each R²⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰1, and R⁷⁰² is independently substituted (e.g. with a substituent group, a size-limited substituent group or a lower substituent group) C₁-C₁₂ alkyl. In embodiments, each R¹⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently unsubstituted C₁-C₁₂ alkyl. In embodiments, each R¹⁰², R²⁰¹, R²⁰², R³⁰¹, R³⁰², R⁴⁰ 1, R⁴⁰², R⁵⁰¹, R⁵⁰², R⁶⁰¹, R⁶⁰², R⁷⁰¹, and R⁷⁰² is independently substituted (e.g. with a substituent group, a size-limited substituent group or a lower substituent group) 2- to 12-membered heteroalkyl. In embodiments, each R¹⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently unsubstituted 2- to 12-membered heteroalkyl.

In embodiments, each s is an integer from 1 to 4. In embodiments, each s is 1. In embodiments, each s is 2. In embodiments, each s is 3. In embodiments, each s is 4.

In embodiments, the cationic lipid of formula (I) is:

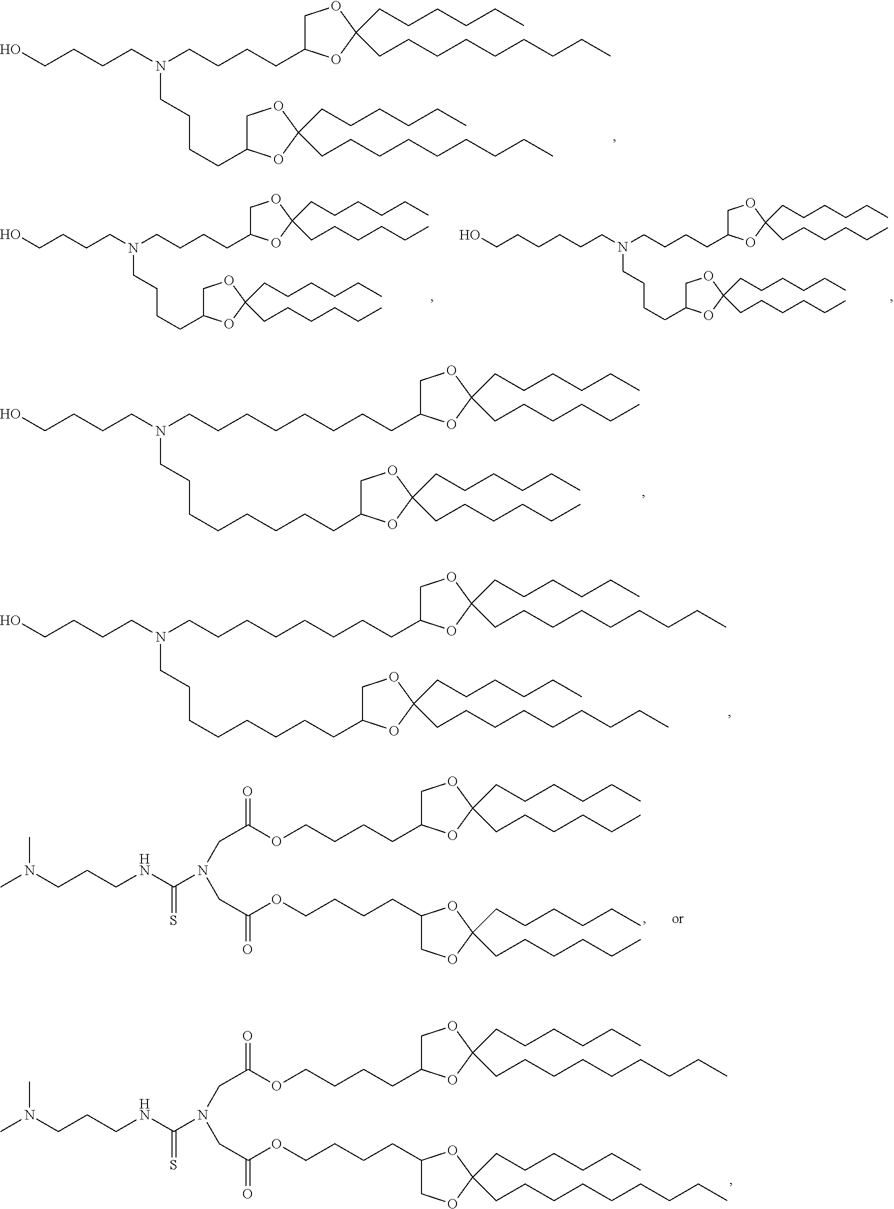

or a pharmaceutically acceptable salt thereof.

In an aspect, provided herein is cationic lipid of formula (II):

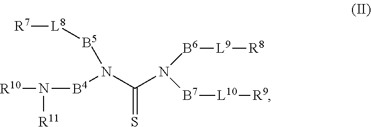

or a pharmaceutically acceptable salt, solvate, hydrate, stereoisomer, or prodrug thereof.
B⁴ is W⁷-L^{a3}-W^{g}, where W⁷ and W⁸ are each independently a bond, substituted or unsubstituted alkylene, or substituted or unsubstituted heteroalkylene, and L^{a3} is a bond, —O(C═O)—, —(C═O)O—, —O(C═O)O—, —C(═O)—, —O—, —O(CR^{a31}R^{a32})ₛO—, —S—, —C(═O)S—, —SC(═O)—, —NR^{a31}C(═O)—, —C(═O)NR^{a31}—, —NR^{a31}C(═O)NR^{a32}—, —NR^{a31}C(═S)—, —C(═S)NR^{a31}, —NR^{a31}C(═S)NR^{a32}, —OC(═O)NR^{a31}, —NR^{a31}C(═O)O—, —SC(═O)NR^{a31}— or —NR^{a31}C(═O)S—.
R¹⁰ and R¹¹ are each independently H, substituted or unsubstituted alkyl, substituted or unsubstituted heteroalkyl, or R¹⁰ and R¹¹ together with the nitrogen atom to which they are connected form a substituted or unsubstituted heterocycloalkyl or substituted or unsubstituted heteroaryl.
B⁵, B⁶, and B⁷ are each independently a bond, substituted or unsubstituted alkylene, or substituted or unsubstituted heteroalkylene.
L⁸ is a bond, —O(C═O)—, —(C═O)O—, —O(C═O)O—, —C(═O)—, —O—, —O(CR⁸⁰¹R⁸⁰²)ₛO—, —S—, —C(═O)S—, —SC(═O)—, —NR⁸⁰¹C(═O)—, —C(═O)NR⁸⁰¹—, —NR⁸⁰¹C(═O)NR⁸⁰²—, —NR⁸⁰¹C(═S)—, —C(═S)NR⁸⁰¹—, —NR⁸⁰¹C(═S)NR⁸⁰²—, —OC(═O)NR⁸⁰¹—, —NR⁸⁰¹C(═O)O—, —SC(═O)NR⁸⁰¹— or —NR⁸⁰¹C(═O)S—.
L⁹ is a bond, —O(C═O)—, —(C═O)O—, —O(C═O)O—, —C(═O)—, —O—, —O(CR⁹⁰¹R⁹⁰²)ₛO—, —S—, —C(═O)S—, —SC(═O)—, —NR⁹⁰¹C(═O)—, —C(═O)NR⁹⁰¹—, —NR⁹⁰¹C(═O)NR⁹⁰²—, —NR⁹⁰¹C(═S)—, —C(═S)NR⁹⁰¹—, —NR⁹⁰¹C(═S)NR⁹⁰²—, —OC(═O)NR⁹⁰¹—, —NR⁹⁰¹C(═O)O—, —SC(═O)NR⁹⁰¹— or —NR⁹⁰¹C(═O)S—.
L¹⁰ is a bond, —O(C═O)—, —(C═O)O—, —O(C═O)O—, —C(═O)—, —O—, —O(CR¹¹⁰R¹¹¹)ₛO—, —S—, —C(═O)S—, —SC(═O)—, —NR¹¹⁰C(═O)—, —C(═O)NR¹¹⁰—, —NR¹¹⁰C(═O)NR¹¹¹—, —NR¹¹⁰C(═S)—, —C(═S)NR¹¹⁰—, —NR¹¹⁰C(═S)NR¹¹¹—, —OC(═O)NR¹¹⁰—, —NR¹¹⁰C(═O)O—, —SC(═O)NR¹¹⁰— or —NR¹¹¹C(═O)S—.
R⁷, R⁸, and R⁹ are each independently H, substituted or unsubstituted C₁-C₃₀ alkyl, or substituted or unsubstituted 2- to 30-membered heteroalkyl.
each R^{a31} and R^{a32} is independently H, substituted or unsubstituted C₁-C₁₂ alkyl, or substituted or unsubstituted 2- to 12-membered heteroalkyl.
each R⁸⁰¹, R⁸⁰², R⁹⁰¹, R⁹⁰², R¹¹⁰, and R¹¹¹ is independently H, substituted or unsubstituted C₁-C₁₂ alkyl, or substituted or unsubstituted 2- to 12-membered heteroalkyl.
each s is independently an integer from 1 to 4.

In embodiments, W⁷ and W⁸ are each independently a bond or substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), or substituted (e.g. with a substituent group, a size-limited substituent group or a lower substituent group) or unsubstituted heteroalkylene (e.g., 2- to 30-membered heteroalkylene, 2- to 8-membered heteroalkylene, or 2- to 4-membered heteroalkylene). In embodiments, W⁷ and W⁸ are each independently substituted with one or more substituent groups. In embodiments, W⁷ and W⁸ are each independently substituted with one or more size-limited substituent groups. In embodiments, W⁷ and W⁸ are each independently substituted with one or more lower substituent groups.

In embodiments, W⁷ and W⁸ are each independently substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, W⁷ and W⁸ are each independently unsubstituted alkylene (e.g., C₁-C₃₀ alkylene, C₁-C₈ alkylene, or C₁-C₄ alkylene). In embodiments, W⁷ and W⁸ are each independently substituted (e.g. with a substituent group, a size-limited substituent group or a lower substituent group) heteroalkylene (e.g., 2- to 30-membered heteroalkylene, 2- to 8-membered heteroalkylene, or 2- to 4-membered heteroalkylene). In embodiments, W⁷ and W⁸ are each independently unsubstituted heteroalkylene (e.g., 2- to 30-membered heteroalkylene, 2- to 8-membered heteroalkylene, or 2- to 4-membered heteroalkylene). In embodiments, W⁷ and W⁸ are each independently a bond.

In embodiments, W⁷ and W⁸ are each independently a bond or substituted or unsubstituted C₁-C₈ alkylene. In embodiments, W⁷ and W^{g} are each independently substituted (e.g. with a substituent group, a size-limited substituent group or a lower substituent group) C₁-C₈ alkylene. In embodiments, W⁷ and W^{g} are each independently unsubstituted C₁-C₈ alkylene.

In embodiments, W⁷ and W⁸ are each independently a bond or substituted or unsubstituted C₂-C₄ alkylene. In embodiments, W⁷ and W⁸ are each independently substituted (e.g. with a substituent group, a size-limited substituent group or a lower substituent group) C₂-C₄ alkylene. In embodiments, W⁷ and W⁸ are each independently unsubstituted C₂-C₄ alkylene.

In embodiments, W⁷ and W⁸ are each independently a bond or unsubstituted C₂-C₄ alkylene. In embodiments, W⁷ and W⁸ are each independently a bond, ethylene, propylene, butylene, substituted (e.g. with a substituent group, a size-limited substituent group or a lower substituent group) ethylene, substituted (e.g. with a substituent group, a size-limited substituent group or a lower substituent group) propylene, or substituted (e.g. with a substituent group, a size-limited substituent group or a lower substituent group) butylene.

In embodiments, L^{a3} is a bond, —O(C═O)—, —(C═O)O—, —O(C═O)O—, or —C(═O)—. In embodiments, L^{a3} is a bond. In embodiments, L^{a3} is —O(C═O)—. In embodiments, L^{a3} is —(C═O)O—. In embodiments, L^{a3} is —O(C═O)O—. In embodiments, L^{a3} is —C(═O)—.

In embodiments, R¹⁰ and R¹¹ are each independently H, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), substituted (e.g. with a substituent group, a size-limited substituent group or a lower substituent group) or unsubstituted heteroalkyl (e.g., 2- to 30-membered heteroalkyl, 2- to 8-membered heteroalkyl, or 2- to 4-membered heteroalkyl), or R¹⁰ and R¹¹ together with the nitrogen atom to which they are connected form a substituted (e.g. with a substituent group, a size-limited substituent group or a lower substituent group) or unsubstituted heterocycloalkyl (e.g., 3- to 8-membered heterocycloalkyl, 3- to 6-membered heterocycloalkyl, or 5- to 6-membered heterocycloalkyl) or substituted (e.g. with a substituent group, a size-limited substituent group or a lower substituent group) or unsubstituted heteroaryl (e.g., 5- to 10-membered heteroaryl, 5- to 9-membered heteroaryl, or 5- to 6-membered heteroaryl). In embodiments, R¹⁰ and R¹¹ are each independently substituted with one or more substituent groups. In embodiments, R¹⁰ and R¹¹ are each independently substituted with one or more size-limited substituent groups. In embodiments, R¹⁰ and R¹¹ are each independently substituted with one or more lower substituent groups.

In embodiments, R¹⁰ and R¹¹ are each independently substituted (e.g. with a substituent group, a size-limited substituent group or a lower substituent group) alkyl (e.g., C₁-C₃₀ alkyl, C₁-C₈ alkyl, or C₁-C₄ alkyl). In embodiments, R¹⁰ and R¹¹ are each independently unsubstituted alkyl (e.g., C₁-C₃₀ alkyl, C₁-C₈ alkyl, or C₁-C₄ alkyl). In embodiments, R¹⁰ and R¹¹ are each independently substituted (e.g. with a substituent group, a size-limited substituent group or a lower substituent group) heteroalkyl (e.g., 2- to 30-membered heteroalkyl, 2- to 8-membered heteroalkyl, or 2- to 4-membered heteroalkyl). In embodiments, R¹⁰ and R¹¹ are each independently unsubstituted heteroalkyl (e.g., 2- to 30-membered heteroalkyl, 2- to 8-membered heteroalkyl, or 2- to 4-membered heteroalkyl). In embodiments, a substituted heterocycloalkyl or substituted heteroaryl formed by the joining of R¹⁰ and R¹¹ groups bonded to the same nitrogen atom is substituted with at least one substituent group, size-limited substituent group, or lower substituent group. If the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R¹⁰ and R¹¹ groups bonded to the same nitrogen atom is substituted, it is substituted with at least one substituent group. In embodiments, when a heterocycloalkyl formed by the joining of R¹⁰ and R¹¹ groups bonded to the same nitrogen atom is substituted, it is substituted with at least one size-limited substituent group. In embodiments, when a heterocycloalkyl formed by the joining of R¹⁰ and R¹¹ groups bonded to the same nitrogen atom is substituted, it is substituted with at least one lower substituent group. In embodiments, when a heteroaryl formed by the joining of R¹⁰ and R¹¹ groups bonded to the same nitrogen atom is substituted, it is substituted with at least one substituent group. In embodiments, when a heteroaryl formed by the joining of R¹⁰ and R¹¹ groups bonded to the same nitrogen atom is substituted, it is substituted with at least one size-limited substituent group. In embodiments, when a heteroaryl formed by the joining of R¹⁰ and R¹¹ groups bonded to the same nitrogen atom is substituted, it is substituted with at least one lower substituent group.

In embodiments, R¹⁰ and R¹¹ together with the nitrogen atom to which they are connected form substituted (e.g. with a substituent group, a size-limited substituent group or a lower substituent group) heterocycloalkyl (e.g., 3- to 8-membered heterocycloalkyl, 3- to 6-membered heterocycloalkyl, or 5- to 6-membered heterocycloalkyl). In embodiments, R¹⁰ and R¹¹ together with the nitrogen atom to which they are connected form unsubstituted heterocycloalkyl (e.g., 3- to 8-membered heterocycloalkyl, 3- to 6-membered heterocycloalkyl. or 5- to 6-membered heterocycloalkyl). In embodiments, R¹⁰ and R¹¹ together with the nitrogen atom to which they are connected form substituted (e.g. with a substituent group, a size-limited substituent group or a lower substituent group) heteroaryl (e.g., 5- to 10-membered heteroaryl, 5- to 9-membered heteroaryl, or 5- to 6-membered heteroaryl). In embodiments, R¹⁰ and R¹¹ together with the nitrogen atom to which they are connected form unsubstituted heteroaryl (e.g., 5- to 10-membered heteroaryl, 5- to 9-membered heteroaryl, or 5- to 6-membered heteroaryl).

In embodiments, R¹⁰ and R¹¹ are each independently H, substituted or unsubstituted alkyl or R¹⁰ and R¹¹ together with the nitrogen atom to which they are connected form a substituted or unsubstituted heterocycloalkyl. In embodiments, R¹⁰ and R¹¹ are each independently H, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl) or R¹⁰ and R¹¹ together with the nitrogen atom to which they are connected form a substituted (e.g. with a substituent group, a size-limited substituent group or a lower substituent group) or unsubstituted heterocycloalkyl. In embodiments, R¹⁰ and R¹¹ are each independently H

In embodiments, R¹⁰ and R¹¹ are each independently substituted or unsubstituted alkyl or R¹⁰ and R¹¹ together with the nitrogen atom to which they are connected form a substituted or unsubstituted heterocycloalkyl. In embodiments, R¹⁰ and R¹¹ are each independently substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl) or R¹⁰ and R¹¹ together with the nitrogen atom to which they are connected form a substituted or unsubstituted heterocycloalkyl (e.g., 3- to 8-membered heterocycloalkyl, 3- to 6-membered heterocycloalkyl, or 5- to 6-membered heterocycloalkyl).

In embodiments, R¹⁰ and R¹¹ are each independently substituted or unsubstituted methyl, ethyl, propyl, isopropyl, butyl, pentyl or hexyl. In embodiments, R¹⁰ and R¹¹ are each independently substituted or unsubstituted methyl, ethyl or propyl.

In embodiments, R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 3- to 8-membered heterocycloalkyl.

In embodiments, R¹⁰ and R¹¹ are each independently substituted or unsubstituted methyl, ethyl, propyl, isopropyl, or R¹⁰ and R¹¹ together with the nitrogen atom to which they are connected form a substituted (e.g. with a substituent group, a size-limited substituent group or a lower substituent group) or unsubstituted 3- to 8-membered heterocycloalkyl.

In embodiments, R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 5- to 6-membered heterocycloalkyl.

In embodiments, R¹⁰ and R¹¹ are each independently substituted or unsubstituted methyl, ethyl, propyl, isopropyl, or R¹⁰ and R¹¹ together with the nitrogen atom to which they are connected form a substituted (e.g. with a substituent group, a size-limited substituent group or a lower substituent group) or unsubstituted 5- to 6-membered heterocycloalkyl.

In embodiments, R¹⁰ and R¹¹ together with the nitrogen atom to which they are connected form a substituted (e.g. with a substituent group, a size-limited substituent group or a lower substituent group) 5- to 6-membered heterocycloalkyl. In embodiments, R¹⁰ and R¹¹ together with the nitrogen atom to which they are connected form unsubstituted 5- to 6-membered heterocycloalkyl.

In embodiments, B⁵, B⁶, and B¹ are each independently a bond, substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), or substituted (e.g. with a substituent group, a size-limited substituent group or a lower substituent group) or unsubstituted heteroalkylene (e.g., 2- to 30-membered heteroalkylene, 2- to 8-membered heteroalkylene, or 2- to 4-membered heteroalkylene). In embodiments, B⁵, B⁶, and B⁷ are each independently substituted with one or more substituent groups. In embodiments, B⁵, B⁶, and B⁷ are each independently substituted with one or more size-limited substituent groups. In embodiments, B⁵, B⁶, and B⁷ are each independently substituted with one or more lower substituent groups.

In embodiments, B⁵, B⁶, and B¹ are each independently a bond. In embodiments, B⁵, B⁶, and B⁷ are each independently substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, B⁵, B⁶, and B⁷ are each independently unsubstituted alkylene (e.g., C₁-C₃₀ alkylene, C₁-C₈ alkylene, or C₁-C₄ alkylene). In embodiments, B⁵, B⁶, and B⁷ are each independently substituted (e.g. with a substituent group, a size-limited substituent group or a lower substituent group) heteroalkylene (e.g., 2- to 30-membered heteroalkylene, 2- to 8-membered heteroalkylene, or 2- to 4-membered heteroalkylene). In embodiments, B⁵, B⁶, and B⁷ are each independently unsubstituted heteroalkylene (e.g., 2- to 30-membered heteroalkylene, 2- to 8-membered heteroalkylene, or 2- to 4-membered heteroalkylene).

In embodiments, B⁵ is a bond.

In embodiments, B⁶ and B⁷ are each independently a bond or substituted or unsubstituted alkylene. In embodiments, B⁶ and B⁷ are each independently a bond or substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene).

In embodiments, B⁶ and B⁷ are each independently a bond or substituted or unsubstituted C₁-C₈ alkylene. In embodiments, B⁶ and B⁷ are each independently a bond or substituted (e.g. with a substituent group, a size-limited substituent group or a lower substituent group) or unsubstituted C₁-C₅ alkylene. In embodiments, B⁶ and B⁷ are each independently a bond. In embodiments, B⁶ and B¹ are each independently substituted (e.g. with a substituent group, a size-limited substituent group or a lower substituent group) C₁-C₈ alkylene. In embodiments, B⁶ and B⁷ are each independently unsubstituted C₁-C₈ alkylene.

In embodiments, B⁶ and B⁷ are each independently a bond or substituted or unsubstituted C₂-C₄ alkylene. In embodiments, B⁶ and B⁷ are each independently a bond or substituted (e.g. with a substituent group, a size-limited substituent group or a lower substituent group) or unsubstituted C₂-C₄ alkylene. In embodiments, B⁶ and B¹ are each independently substituted (e.g. with a substituent group, a size-limited substituent group or a lower substituent group) C₂-C₄ alkylene. In embodiments, B⁶ and B⁷ are each independently unsubstituted C₂-C₄ alkylene.

In embodiments, B⁶ and B⁷ are each independently a bond or unsubstituted C₂-C₄ alkylene. In embodiments, B⁶ and B⁷ are each independently a bond, ethylene, propylene, or butylene. In embodiments, B⁶ and B¹ are each independently a bond. In embodiments, B⁶ and B⁷ are each independently ethylene. In embodiments, B⁶ and B⁷ are each independently propylene. In embodiments, B⁶ and B⁷ are each independently butylene.

In embodiments, L⁸ is a bond, —O(C═O)—, —(C═O)O—, or —C(═O)—. In embodiments, L⁸ is a bond. In embodiments, L⁸ is —O(C═O)—. In embodiments, L⁸ is —(C═O)O—. In embodiments, L⁸ is —C(═O)—.

In embodiments, L⁹ is a bond, —O(C═O)—, —(C═O)O—, or —C(═O)—. In embodiments, L⁹ is —O(C═O)— or —(C═O)O—. In embodiments, L⁹ is a bond. In embodiments, L⁹ is —O(C═O)—. In embodiments, L⁹ is —(C═O)O—. In embodiments, L⁹ is —C(═O)—.

In embodiments, L¹⁰ is a bond, —O(C═O)—, —(C═O)O—, or —C(═O)—. In embodiments, L¹⁰ is —O(C═O)— or —(C═O)O—. In embodiments, L¹⁰ is a bond. In embodiments, L¹⁰ is —O(C═O)—. In embodiments, L¹⁰ is —(C═O)O—. In embodiments, L¹⁰ is —C(═O)—.

In embodiments, R⁷, R⁸, and R⁹ are each independently H, substituted (e.g. with a substituent group, a size-limited substituent group or a lower substituent group) or unsubstituted C₁-C₃₀ alkyl, or substituted (e.g. with a substituent group, a size-limited substituent group or a lower substituent group) or unsubstituted 2- to 30-membered heteroalkyl. In embodiments, R⁷, R⁸, and R⁹ are each independently substituted with one or more substituent groups. In embodiments, R⁷, R⁸, and R⁹ are each independently substituted with one or more size-limited substituent groups. In embodiments, R⁷, R⁸, and R⁹ are each independently substituted with one or more lower substituent groups.

In embodiments, R⁷, R⁸, and R⁹ are each independently H. In embodiments, R⁷, R⁸, and R⁹ are each independently substituted (e.g. with a substituent group, a size-limited substituent group or a lower substituent group) C₁-C₃₀ alkyl. In embodiments, R⁷, R⁸, and R⁹ are each independently unsubstituted C₁-C₃ alkyl. In embodiments, R⁷, R⁸, and R⁹ are each independently substituted (e.g. with a substituent group, a size-limited substituent group or a lower substituent group) 2- to 30-membered heteroalkyl. In embodiments, R⁷, R⁸, and R⁹ are each independently unsubstituted 2- to 30-membered heteroalkyl.

In embodiments, R⁷, R⁸, and R⁹ are each independently H or substituted or unsubstituted C₁-C₃₀ alkyl. In embodiments, R⁷, R⁸, and R⁹ are each independently H or substituted (e.g. with a substituent group, a size-limited substituent group or a lower substituent group) or unsubstituted C₁-C₃₀ alkyl. In embodiments, R⁷, R⁸, and R⁹ are each independently substituted (e.g. with a substituent group, a size-limited substituent group or a lower substituent group) or unsubstituted C₁-C₃₀ alkyl.

In embodiments, R⁷, R⁸, and R⁹ are each independently substituted or unsubstituted C₁-C₂₀ alkyl. In embodiments, R⁷, R⁸, and R⁹ are each independently substituted (e.g. with a substituent group, a size-limited substituent group or a lower substituent group) or unsubstituted C₁-C₂₀ alkyl. In embodiments, R⁷, R⁸, and R⁹ are each independently substituted (e.g. with a substituent group, a size-limited substituent group or a lower substituent group) C₁-C₂₀ alkyl. In embodiments, R⁷, R⁸, and R⁹ are each independently unsubstituted C₁-C₂₀ alkyl.

In embodiments, each R^{a31} and R^{a32} is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2- to 12-membered heteroalkyl. In embodiments, each R^{a31} and R^{a32} is independently H. In embodiments, each R^{a3}i and R^{a32} is independently substituted (e.g. with a substituent group, a size-limited substituent group or a lower substituent group) C₁-C₁₂ alkyl. In embodiments, each R^{a31} and R^{a32} is independently unsubstituted C₁-C₁₂ alkyl. In embodiments, each R^{a31} and R^{a32} is independently substituted (e.g. with a substituent group, a size-limited substituent group or a lower substituent group) 2- to 12-membered heteroalkyl. In embodiments, each R^{a31} and R^{a32} is independently unsubstituted 2- to 12-membered heteroalkyl.

In embodiments, each R⁸⁰¹, R⁸⁰², R⁹⁰¹, R⁹⁰², R¹¹⁰, and R¹¹¹ is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2- to 12-membered heteroalkyl. In embodiments, each R⁸⁰¹, R⁸⁰², R⁹⁰¹, R⁹⁰², R¹¹⁰, and R¹¹¹ is independently H. In embodiments, each R⁸⁰¹, R⁸⁰², R⁹⁰¹, R⁹⁰², R¹¹⁰, and R¹¹¹ is independently substituted (e.g. with a substituent group, a size-limited substituent group or a lower substituent group) C₁-C₁₂ alkyl. In embodiments, each R⁸⁰¹, R⁸⁰², R⁹⁰¹, R⁹⁰², R¹¹⁰, and R¹¹¹ is independently unsubstituted C₁-C₁₂ alkyl. In embodiments, each R⁸⁰¹, R⁸⁰², R⁹⁰¹, R⁹⁰², R¹¹⁰, and R¹¹¹ is independently substituted (e.g. with a substituent group, a size-limited substituent group or a lower substituent group) 2- to 12-membered heteroalkyl. In embodiments, each R⁸⁰¹, R⁸⁰², R⁹⁰¹, R⁹⁰², R¹¹⁰, and R¹¹¹ is independently unsubstituted 2- to 12-membered heteroalkyl.

In embodiments, each s is independently an integer from 1 to 4. In embodiments, each s is 1. In embodiments, each s is 2. In embodiments, each s is 3. In embodiments, each s is 4.

In embodiments, W⁷ and W⁸ are each independently a bond or substituted or unsubstituted alkylene. L^{a3} is a bond.
R¹⁰ and R¹¹ are each independently H, substituted or unsubstituted alkyl or R¹⁰ and R¹¹ together with the nitrogen atom to which they are connected form a substituted or unsubstituted heterocycloalkyl. B⁵ is a bond. B⁶ and B⁷ are each independently a bond or substituted or unsubstituted alkylene. L⁸ is a bond. L⁹ is a bond, —O(C═O)—, —(C═O)O—, or —C(═O)—.
L¹⁰ is a bond, —O(C═O)—, —(C═O)O—, or —C(═O)—, and R⁷, R⁸, and R⁹ are each independently H or substituted or unsubstituted C₁-C₃₀ alkyl.

In embodiments, W⁷ and W⁸ are each independently a bond or substituted or unsubstituted C₁-C₈ alkylene. L^{a3} is a bond.
R¹⁰ and R¹¹ are each independently substituted or unsubstituted alkyl or R¹⁰ and R¹¹ together with the nitrogen atom to which they are connected form a substituted or unsubstituted heterocycloalkyl. B⁵ is a bond. B⁶ and B⁷ are each independently a bond or substituted or unsubstituted C₁-C₈ alkylene. L⁸ is a bond. L⁹ is —O(C═O)— or —(C═O)O—. L¹⁰-O(C═O)— or —(C═O)O—, and R⁷, R⁸, and R⁹ are each independently substituted or unsubstituted C₁-C₂₀ alkyl.

In embodiments, W⁷ and W⁸ are each independently a bond or substituted or unsubstituted C₂-C₄ alkylene. L^{a3} is a bond.
R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 3- to 8-membered heterocycloalkyl. B⁵ is a bond. B⁶ and B⁷ are each independently a bond or substituted or unsubstituted C₂-C₄ alkylene. L⁸ is a bond. L⁸ is —O(C═O)— or —(C═O)O—.
L¹⁰-O(C═O)— or —(C═O)O—. R⁷ is H or methyl, and R⁸, and R⁹ are each independently substituted or unsubstituted C₁-C₂₀ alkyl.

In embodiments, W⁷ and W⁸ are each independently a bond or unsubstituted C₂-C₄ alkylene. Las is a bond.
R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 5- to 6-membered heterocycloalkyl. B⁵ is a bond. B⁶ and B⁷ are each independently a bond or unsubstituted C₂-C₄ alkylene. L⁸ is a bond. L⁸ is —O(C═O)— or —(C═O)O—. L¹⁰ is —O(C═O)— or —(C═O)O—. R⁷ is H or methyl, and R′ and R′ are each independently substituted or unsubstituted C₁-C₂₀ alkyl.

In embodiments, W⁷ and W⁸ are each independently a bond or unsubstituted C₂-C₄ alkylene. Las is a bond.

R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 5- to 6-membered heterocycloalkyl. B⁵, B⁶, and B⁷ are each independently a bond. L⁸ is a bond. L⁹ is a bond. L¹⁰ is a bond. R⁷ is H or methyl, and R⁸ and R⁹ are each independently substituted or unsubstituted C₁-C₃₀ alkyl.

In embodiments, the cationic lipid of formula (II) is:

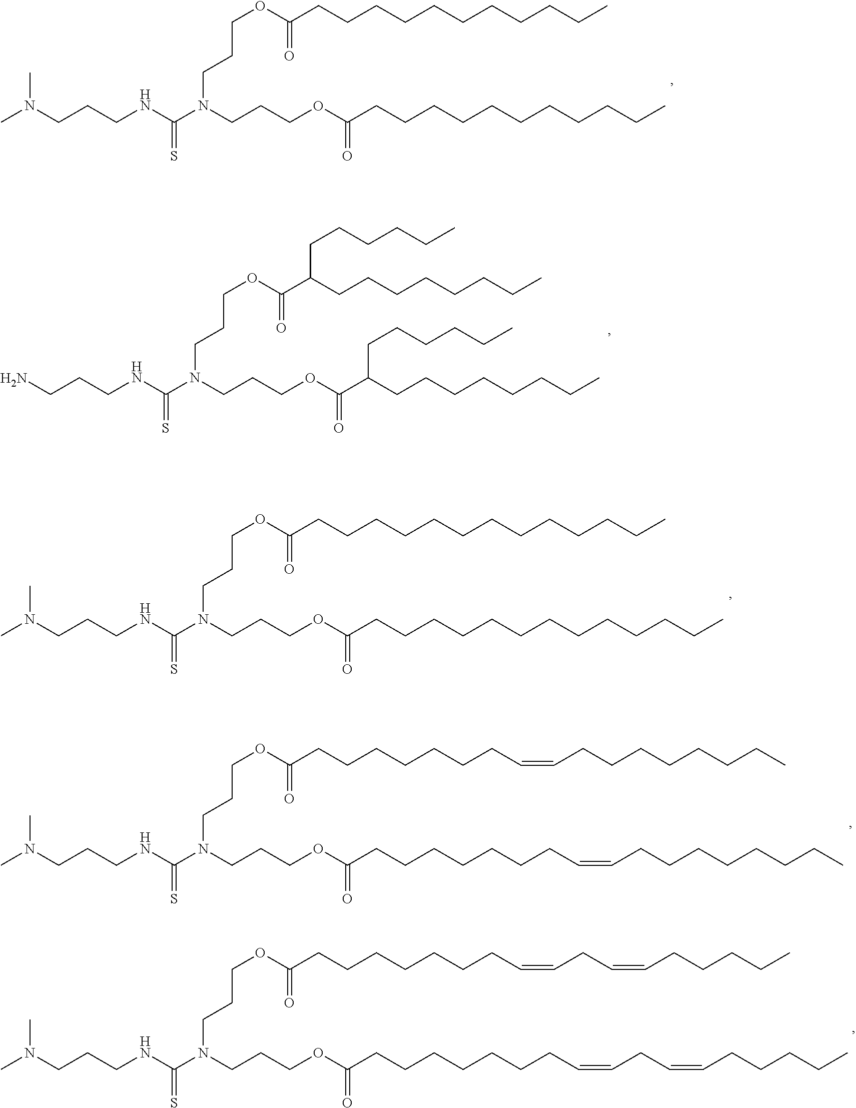

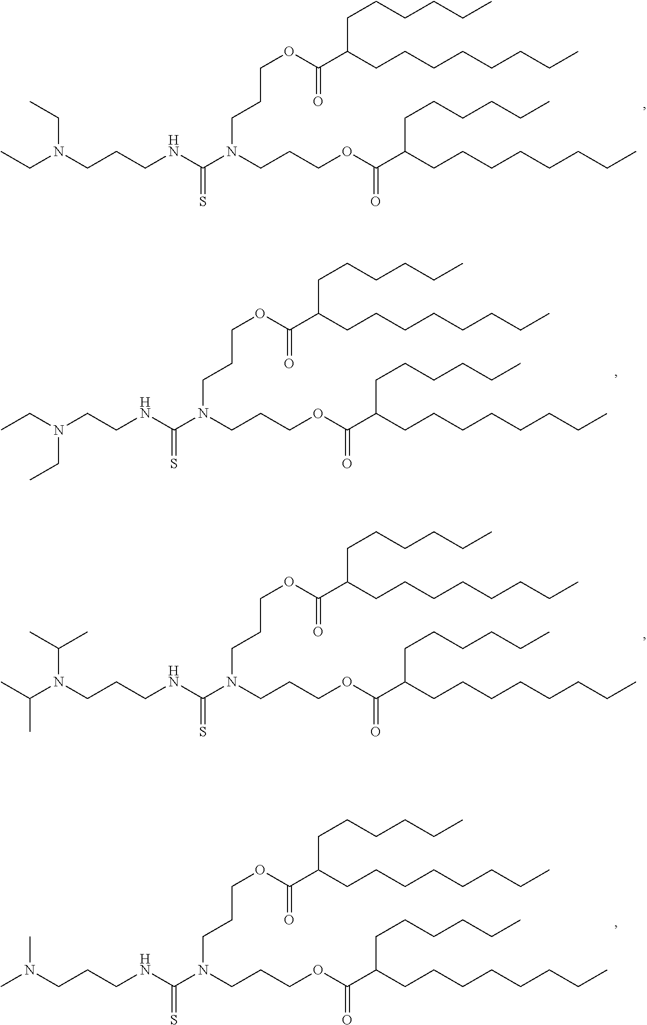

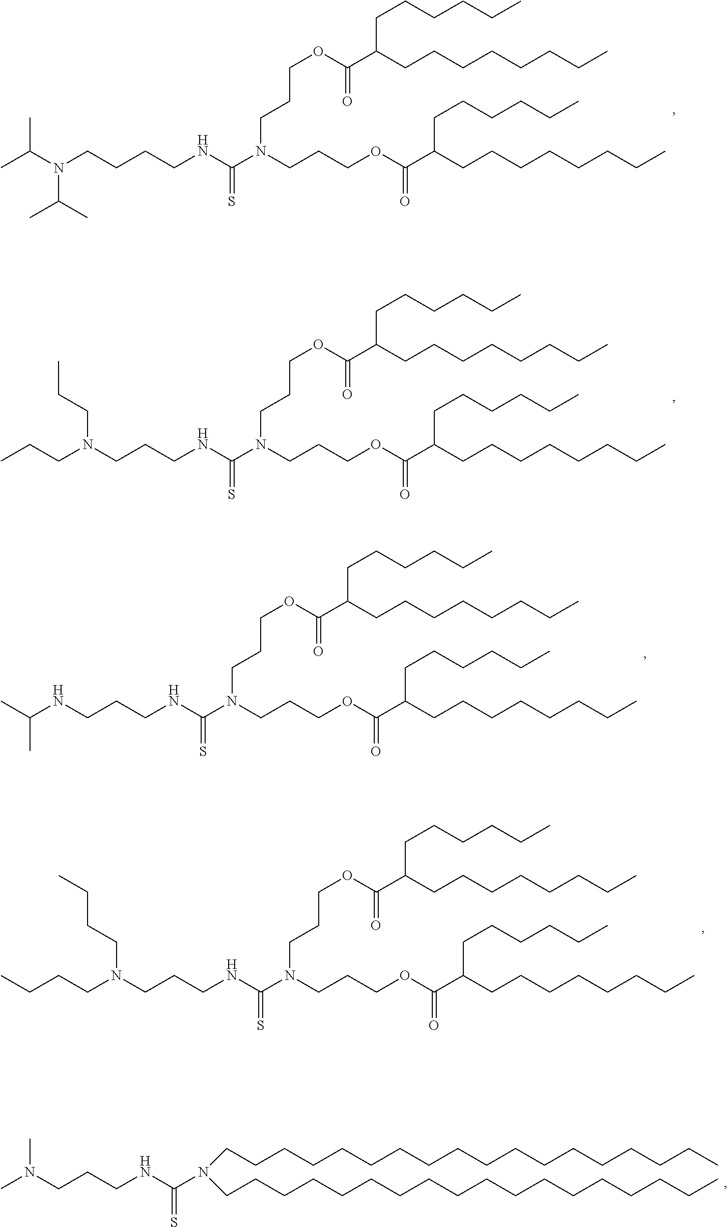

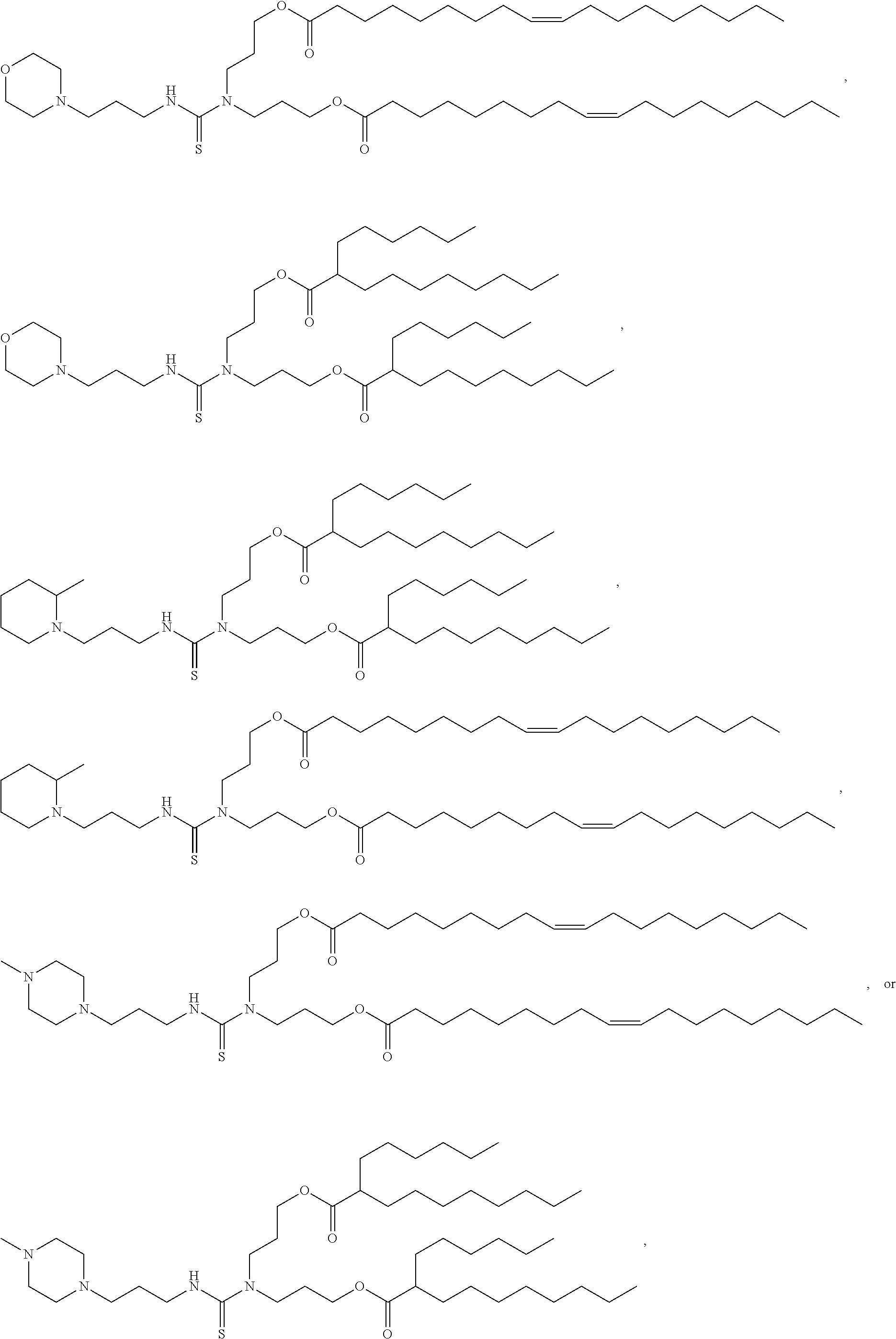

or a pharmaceutically acceptable salt thereof.

In an aspect, provided herein is cationic lipid of formula (III):

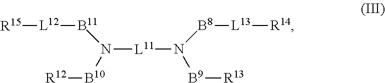

or a pharmaceutically acceptable salt, solvate, hydrate, stereoisomer, or prodrug thereof.

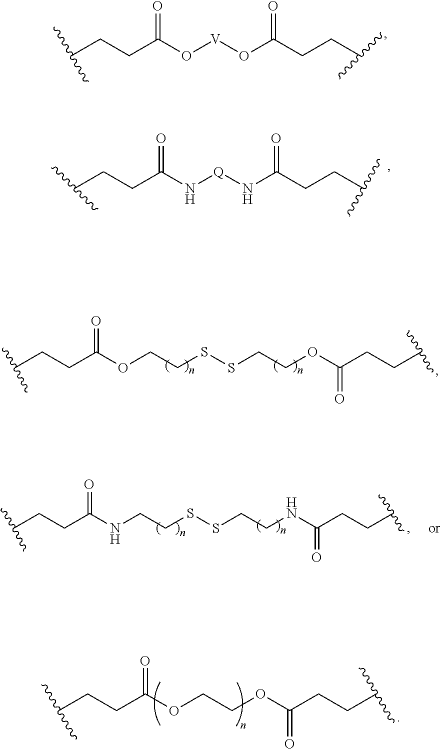

L¹¹ is
Q is substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene.
V is substituted or unsubstituted alkylene, substituted or unsubstituted cycloalkylene, substituted or unsubstituted arylene.
B⁸, B⁹, B¹⁰, and B¹¹ are each independently a bond, substituted or unsubstituted alkylene, or substituted or unsubstituted heteroalkylene.
L¹² is a bond, —O(C═O)—, —(C═O)O—, —O(C═O)O—, —C(═O)—, —O—, —O(CR²¹⁰R²¹¹)ₛO—, —S—, —C(═O)S—, —SC(═O)—, —NR²¹⁰C(═O)—, —C(═O)NR²¹⁰—, —NR²¹⁰C(═O)NR²¹¹—, —NR²¹⁰C(═S)—, —C(═S)NR²¹⁰, —NR²¹⁰C(═S)NR²¹¹—, —OC(═O)NR²¹⁰—, —NR²¹⁰C(═O)O—, —SC(═O)NR²¹⁰— or —NR²¹⁰C(═O)S—.
L¹³ is a bond, —O(C═O)—, —(C═O)O—, —O(C═O)O—, —C(═O)—, —O—, —O(CR³¹⁰R³¹¹)SO—, —S—, —C(═O)S—, —SC(═O)—, —NR³¹⁰C(═O)—, —C(═O)NR³¹⁰—, —NR³¹⁰C(═O)NR³¹¹—, —NR³¹⁰C(═S)—, —C(═S)NR³¹⁰—, —NR³¹⁰C(═S)NR³¹¹—, —OC(═O)NR³¹⁰—, —NR³¹⁰C(═O)O—, —SC(═O)NR³¹⁰— or —NR³¹⁰C(═O)S—.
R¹² is H, —OR^{12A}, —SR^{12A}, —NR^{12A}, —CN, —(C═O)R^{12A}, —O(C═O)R^{12A}, (C═O)OR^{12A}, —NR^{12A}(C═O)—R^{12B}, —(C═O)NR^{12A}R^{12B}.
R¹³ is H, —OR^{13A}, —SR^{13A}, —NR^{13A}, —CN, —(C═O)R^{13A}, —O(C═O)R^{13A}, —(C═O)OR^{13A}, —NR^{13A}(C═O)—R^{13B}, —(C═O)NR^{13A}R^{13B}.
R¹⁴ and R¹⁵ are each independently substituted or unsubstituted C₂-C₃₀ alkyl, or substituted or unsubstituted 2- to 30-membered heteroalkyl.
R^{12A}, R^{12B}, R^{13A}, and R^{13B} are each independently H, substituted or unsubstituted C₁-C₂₀ alkyl, or substituted or unsubstituted 2- to 20-membered heteroalkyl.
each R²¹⁰, R²¹¹, R³¹⁰, and R³¹¹ is independently H, substituted or unsubstituted C₁-C₁₂ alkyl, or substituted or unsubstituted 2- to 12-membered heteroalkyl.
each n is independently an integer from 0 to 8, and
each s is independently an integer from 1 to 4.

In embodiments, L¹¹ is

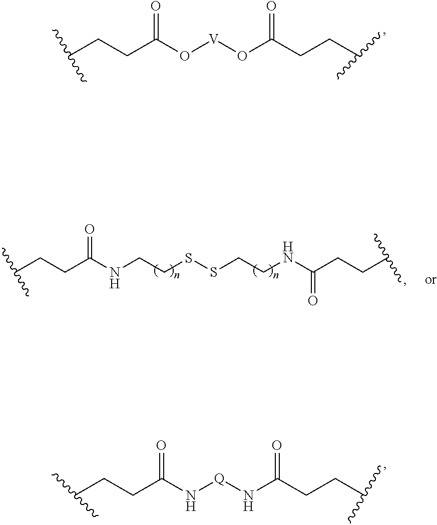

where n is an integer from 0 to 8, V is substituted or unsubstituted alkylene, and Q is substituted or unsubstituted alkylene.

In embodiments, L¹¹ is

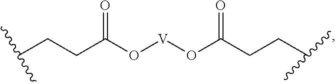

where V is substituted or unsubstituted alkylene. In embodiments, L¹¹ is

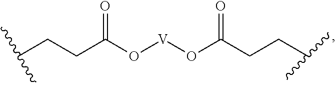

where V is substituted (e.g., with a substituent group, a size-limited substituent group or a lower substituent group) or unsubstituted alkylene (e.g., C₁-C₃₀ alkylene, C₁-C₈ alkylene, or C₁-C₄ alkylene).

In embodiments, L¹¹ is

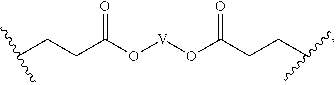

where V is substituted (e.g., with a substituent group, a size-limited substituent group or a lower substituent group) alkylene (e.g., C₁-C₃₀ alkylene, C₁-C₈ alkylene, or C₁-C₄ alkylene). In embodiments, L¹¹ is

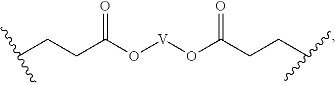

where V is unsubstituted alkylene (e.g., C₁-C₃₀ alkylene, C₁-C₈ alkylene, or C₁-C₄ alkylene).

In embodiments, L¹¹ is

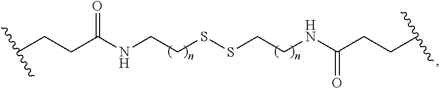

where n is an integer from 0 to 8. In embodiments, L¹¹ is

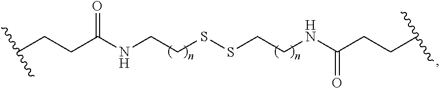

where n is an integer from 0 to 4.

In embodiments, L¹¹ is

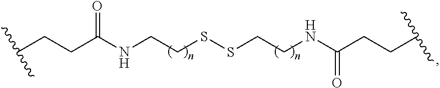

where n is 0. In embodiments, L¹¹ is

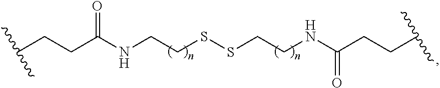

where n is 1.

In embodiments, L¹¹ is

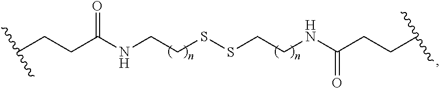

where n is 2.

In embodiments, L¹¹ is

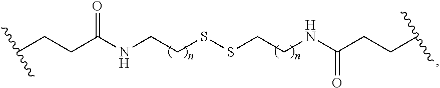

where n is 3.

In embodiments, L¹¹ is

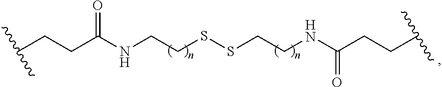

where n is 4.

In embodiments, L¹¹ is

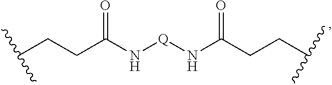

where Q is substituted or unsubstituted alkylene. In embodiments, L¹¹ is

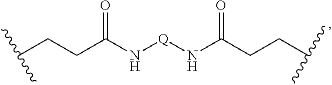

where Q is substituted (e.g., with a substituent group, a size-limited substituent group or a lower substituent group) or unsubstituted alkylene (e.g., C₁-C₃₀ alkylene, C₁-C₈ alkylene, or C₁-C₄ alkylene).

In embodiments, L¹¹ is

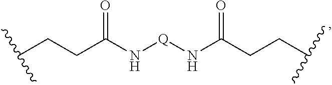

where Q is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, L¹¹ is

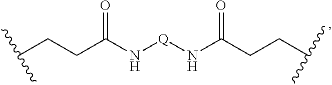

where Q is unsubstituted alkylene (e.g., C₁-C₃₀ alkylene, C₁-C₈ alkylene, or C₁-C₄ alkylene).

In embodiments, L¹¹ is

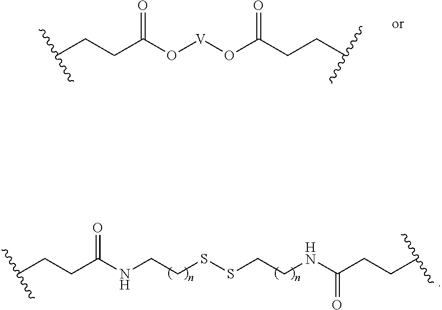

In embodiments, Q is substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), substituted (e.g. with a substituent group, a size-limited substituent group or a lower substituent group) or unsubstituted heteroalkylene (e.g., 2- to 30-membered heteroalkylene, 2- to 8-membered heteroalkylene, or 2- to 4-membered heteroalkylene), substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene), substituted (e.g. with a substituent group, a size-limited substituent group or a lower substituent group) or unsubstituted heterocycloalkylene (e.g., 3- to 8-membered heterocycloalkylene, 3- to 6-membered heterocycloalkylene, or 5- to 6-membered heterocycloalkylene), substituted (e.g. with a substituent group, a size-limited substituent group or a lower substituent group) or unsubstituted arylene (e.g., C₆-C₁₀ arylene, C₁₀ arylene, or phenylene), or substituted (e.g. with a substituent group, a size-limited substituent group or a lower substituent group) or unsubstituted heteroarylene (e.g., 5- to 10-membered heteroarylene, 5- to 9-membered heteroarylene, or 5- to 6-membered heteroarylene). In embodiments, Q is substituted with one or more substituent groups. In embodiments, Q is substituted with one or more size-limited substituent groups. In embodiments, Q is substituted with one or more lower substituent groups.

In embodiments, Q is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, Q is unsubstituted alkylene (e.g., C₁-C₃₀ alkylene, C₁-C₈ alkylene, or C₁-C₄ alkylene). In embodiments, Q is substituted (e.g. with a substituent group, a size-limited substituent group or a lower substituent group) heteroalkylene (e.g., 2- to 30-membered heteroalkylene, 2- to 8-membered heteroalkylene, or 2- to 4-membered heteroalkylene). In embodiments, Q is unsubstituted heteroalkylene (e.g., 2- to 30-membered heteroalkylene, 2- to 8-membered heteroalkylene, or 2- to 4-membered heteroalkylene). In embodiments, Q is substituted (e.g. with a substituent group, a size-limited substituent group or a lower substituent group) cycloalkylene (e.g., C₃-C₈ cycloalkylene, C₃-C₆ cycloalkylene, or C₅-C₆ cycloalkylene). In embodiments, Q is unsubstituted cycloalkylene (e.g., C₃-C₈ cycloalkylene, C₃-C₆ cycloalkylene, or C₅-C₆ cycloalkylene). In embodiments, Q is substituted (e.g. with a substituent group, a size-limited substituent group or a lower substituent group) heterocycloalkylene (e.g., 3- to 8-membered heterocycloalkylene, 3- to 6-membered heterocycloalkylene, or 5- to 6-membered heterocycloalkylene). In embodiments, Q is unsubstituted heterocycloalkylene (e.g., 3- to 8-membered heterocycloalkylene, 3- to 6-membered heterocycloalkylene, or 5- to 6-membered heterocycloalkylene). In embodiments, Q is substituted (e.g. with a substituent group, a size-limited substituent group or a lower substituent group) arylene (e.g., C₆-C₁₀ arylene, C₁₀ arylene, or phenylene). In embodiments, Q is unsubstituted arylene (e.g., C₆-C₁₀ arylene, C₁₀ arylene, or phenylene). In embodiments, Q is substituted (e.g. with a substituent group, a size-limited substituent group or a lower substituent group) heteroarylene (e.g., 5- to 10-membered heteroarylene, 5- to 9-membered heteroarylene, or 5- to 6-membered heteroarylene). In embodiments, Q is unsubstituted heteroarylene (e.g., 5- to 10-membered heteroarylene, 5- to 9-membered heteroarylene, or 5- to 6-membered heteroarylene).

In embodiments, Q is substituted or unsubstituted alkylene. In embodiments, Q is substituted (e.g. with a substituent group, a size-limited substituent group or a lower substituent group) alkylene (e.g., C₁-C₃₀ alkylene, C₁-C₈ alkylene, or C₁-C₄ alkylene).

In embodiments, V is substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene), or substituted (e.g. with a substituent group, a size-limited substituent group or a lower substituent group) or unsubstituted arylene (e.g., C₆-C₁₀ arylene, C₁₀ arylene, or phenylene). In embodiments, V is substituted with one or more substituent groups. In embodiments, V is substituted with one or more size-limited substituent groups. In embodiments, V is substituted with one or more lower substituent groups.

In embodiments, V is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, V is unsubstituted alkylene (e.g., C₁-C₃₀ alkylene, C₁-C₈ alkylene, or C₁-C₄ alkylene). In embodiments, V is substituted (e.g. with a substituent group, a size-limited substituent group or a lower substituent group) cycloalkylene (e.g., C₃-C₈ cycloalkylene, C₃-C₆ cycloalkylene, or C₅-C₆ cycloalkylene). In embodiments, V is unsubstituted cycloalkylene (e.g., C₃-C₈ cycloalkylene, C₃-C₆ cycloalkylene, or C₅-C₆ cycloalkylene). In embodiments, V is substituted (e.g. with a substituent group, a size-limited substituent group or a lower substituent group) arylene (e.g., C₆-C₁₀ arylene, C₁₀ arylene, or phenylene). In embodiments, V is unsubstituted arylene (e.g., C₆-C₁₀ arylene, C₁₀ arylene, or phenylene).

In embodiments, V is substituted or unsubstituted alkylene. In embodiments, V is substituted (e.g. with a substituent group, a size-limited substituent group or a lower substituent group) alkylene (e.g., C₁-C₃₀ alkylene, C₁-C₈ alkylene, or C₁-C₄ alkylene).

In embodiments, B⁸, B⁹, B¹⁰, and B¹¹ are each independently a bond, substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), or substituted (e.g. with a substituent group, a size-limited substituent group or a lower substituent group) or unsubstituted heteroalkylene (e.g., 2- to 30-membered heteroalkylene, 2- to 8-membered heteroalkylene, or 2- to 4-membered heteroalkylene). In embodiments, B⁸, B⁹, B¹⁰, and B¹¹ are each independently substituted with one or more substituent groups. In embodiments, B⁸, B⁹, B¹⁰, and B¹¹ are each independently substituted with one or more size-limited substituent groups. In embodiments, B⁸, B⁹, B¹⁰, and B¹¹ are each independently substituted with one or more lower substituent groups.

In embodiments, B⁸, B⁹, B¹⁰, and B¹′ are each independently a bond. In embodiments, B⁸, B⁹, B¹⁰, and B¹¹ are each independently substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, B⁸, B⁹, B¹⁰, and B¹¹ are each independently unsubstituted alkylene (e.g., C₁-C₃₀ alkylene, C₁-C₈ alkylene, or C₁-C₄ alkylene). In embodiments, B⁸, B⁹, B¹⁰, and B¹¹ are each independently substituted (e.g. with a substituent group, a size-limited substituent group or a lower substituent group) heteroalkylene (e.g., 2- to 30-membered heteroalkylene, 2- to 8-membered heteroalkylene, or 2- to 4-membered heteroalkylene). In embodiments, B⁸, B⁹, B¹⁰, and B¹¹ are each independently unsubstituted heteroalkylene (e.g., 2- to 30-membered heteroalkylene, 2- to 8-membered heteroalkylene, or 2- to 4-membered heteroalkylene).

In embodiments, B⁸, B⁹, B¹⁰, and B¹¹ are each independently substituted or unsubstituted alkylene. In embodiments, B⁸, B⁹, B¹⁰, and B¹¹ are each independently substituted (e.g. with a substituent group, a size-limited substituent group or a lower substituent group) or unsubstituted alkylene (e.g. with a substituent group, a size-limited substituent group or a lower substituent group).

In embodiments, B⁸, B⁹, B¹⁰, and B¹¹ are each independently substituted or unsubstituted C₁-C₂₀ alkylene. In embodiments, B⁸, B⁹, B¹⁰, and B¹¹ are each independently substituted (e.g. with a substituent group, a size-limited substituent group or a lower substituent group) or unsubstituted C₁-C₂₀ alkylene. In embodiments, B⁸, B⁹, B¹⁰, and B¹¹ are each independently substituted (e.g. with a substituent group, a size-limited substituent group or a lower substituent group) C₁-C₂₀ alkylene. In embodiments, B⁸, B⁹, B¹⁰, and B¹¹ are each independently unsubstituted C₁-C₂₀ alkylene.

In embodiments, B⁸, B⁹, B¹⁰, and B¹¹ are each independently substituted or unsubstituted C₁-C₈ alkylene. In embodiments, B⁸, B⁹, B¹⁰, and B¹¹ are each independently substituted (e.g. with a substituent group, a size-limited substituent group or a lower substituent group) or unsubstituted C₁-C₈ alkylene. In embodiments, B⁸, B⁹, B¹⁰, and B¹¹ are each independently substituted (e.g. with a substituent group, a size-limited substituent group or a lower substituent group) C₁-C₈ alkylene. In embodiments, B⁸, B⁹, B¹⁰, and B¹¹ are each independently unsubstituted C₁-C₈ alkylene.

In embodiments, L¹² is a bond, —O(C═O)—, —(C═O)O—, —O(C═O)O—, —C(═O)—, or —O—. In embodiments, L¹² is a bond. In embodiments, L¹² is —O(C═O)—. In embodiments, L¹² is —(C═O)O—. In embodiments, L¹² is —O(C═O)O—. In embodiments, L¹² is —C(═O)—. In embodiments, L¹² is —O—.

In embodiments, L¹² is —O(C═O)— or —(C═O)O—.

In embodiments, L¹³ is a bond, —O(C═O)—, —(C═O)O—, —O(C═O)O—, —C(═O)—, or —O—. In embodiments, L¹³ is a bond. In embodiments, L¹³ is —O(C═O)—. In embodiments, L¹³ is —(C═O)O—. In embodiments, L³ is —O(C═O)O—. In embodiments, L¹³ is —C(═O)—. In embodiments, L¹³ is —O—.

In embodiments, L¹³ is —O(C═O)— or —(C═O)O—.

In embodiments, R¹² is H, —OR^{12A}, —SR^{12A}, —NR^{12A}, —CN, or —(C═O)R^{12A}. In embodiments, R¹² is H, —OR^{12A}, or —NR^{12A}. In embodiments, R¹² is H or —OR^{12A}.

In embodiments, R¹² is H. In embodiments, R¹² is —OR^{12A}. In embodiments, R¹² is —SR^{12A}In embodiments, R¹² is —NR^{12A}. In embodiments, R¹² is CN. In embodiments, R¹² is —(C═O)R^{12A}.

In embodiments, R¹² is —OH, methoxy, or ethoxy.

In embodiments, R¹³ is H, —OR^{13A}, —SR^{13A}, —NR^{13A}, —CN, or —(C═O)R^{13A}. In embodiments, R¹³ is H, —OR^{13A}, or —NR^{13A}. In embodiments, R¹³ is H or —OR^{13A}.

In embodiments, R¹³ is H. In embodiments, R¹³ is —OR^{13A}. In embodiments, R¹³ is —SR^{13A}. In embodiments, R¹³ is —NR^{13A} In embodiments, R¹³ is CN. In embodiments, R¹³ is —(C═O)R^{13A}.

In embodiments, R¹³ is —OH, methoxy, or ethoxy.

In embodiments, R^{12A} and R^{13A} are each independently H, substituted (e.g. with a substituent group, a size-limited substituent group or a lower substituent group) or unsubstituted C₁-C₂₀ alkyl, or substituted (e.g. with a substituent group, a size-limited substituent group or a lower substituent group) or unsubstituted 2- to 30-membered heteroalkyl. In embodiments, R^{12A} and R^{13A} are each independently substituted with one or more substituent groups. In embodiments, R^{12A} and R^{13A} are each independently substituted with one or more size-limited substituent groups. In embodiments, R^{12A} and R^{13A} are each independently substituted with one or more lower substituent groups.

In embodiments, R^{12A} and R^{13A} are each independently H. In embodiments, R^{12A} and R^{13A} are each independently substituted (e.g. with a substituent group, a size-limited substituent group or a lower substituent group) C₁-C₂₀ alkyl. In embodiments, R^{12A} and R^{13A} are each independently unsubstituted C₁-C₂₀ alkyl. In embodiments, R^{12A} and R^{13A} are each independently substituted (e.g. with a substituent group, a size-limited substituent group or a lower substituent group) 2- to 30-membered heteroalkyl. In embodiments, R^{12A} and R^{13A} are each independently unsubstituted 2- to 30-membered heteroalkyl.

In embodiments, R^{12A} and R^{13A} are each independently H, substituted or unsubstituted C₁-C₂₀ alkyl. In embodiments, R^{12A} and R^{3A} are each independently H, substituted (e.g. with a substituent group, a size-limited substituent group or a lower substituent group) or unsubstituted C₁-C₂₀ alkyl. In embodiments, R^{12A} and R^{13A} are each independently substituted (e.g. with a substituent group, a size-limited substituent group or a lower substituent group) C₁-C₂₀ alkyl. In embodiments, R^{12A} and R^{13A} are each independently unsubstituted C₁-C₂₀ alkyl.

In embodiments, R^{12A} and R^{13A} are each independently H, substituted or unsubstituted C₁-C₈ alkyl. In embodiments, R^{12A} and R^{13A} are each independently H, substituted (e.g. with a substituent group, a size-limited substituent group or a lower substituent group) or unsubstituted C₁-C₈ alkyl. In embodiments, R^{12A} and R^{13A} are each independently substituted (e.g. with a substituent group, a size-limited substituent group or a lower substituent group) C₁-C₈ alkyl. In embodiments, R^{12A} and R^{13A} are each independently unsubstituted C₁-C₈alkyl.

In embodiments, R^{12B} and R^{13B} are each independently H, substituted (e.g. with a substituent group, a size-limited substituent group or a lower substituent group) or unsubstituted C₁-C₂₀ alkyl, or substituted (e.g. with a substituent group, a size-limited substituent group or a lower substituent group) or unsubstituted 2- to 30-membered heteroalkyl.

In embodiments, R^{12B} and R^{13B} are each independently H. In embodiments, R^{12B} and R^{13B} are each independently substituted (e.g. with a substituent group, a size-limited substituent group or a lower substituent group) CL-C₂₀ alkyl. In embodiments, R^{12B} and R^{13B} are each independently unsubstituted C₁-C₂₀ alkyl. In embodiments, R^{12B} and R^{13B} are each independently substituted (e.g. with a substituent group, a size-limited substituent group or a lower substituent group) 2- to 30-membered heteroalkyl. In embodiments, R^{12B} and R^{13B} are each independently unsubstituted 2- to 30-membered heteroalkyl.

In embodiments, R¹⁴ and R¹⁵ are each independently substituted (e.g. with a substituent group, a size-limited substituent group or a lower substituent group) or unsubstituted C₂-C₃₀ alkyl, or substituted (e.g. with a substituent group, a size-limited substituent group or a lower substituent group) or unsubstituted 2- to 30-membered heteroalkyl. In embodiments, R¹⁴ and R¹⁵ are each independently substituted with one or more substituent groups. In embodiments, R¹⁴ and R¹⁵ are each independently substituted with one or more size-limited substituent groups. In embodiments, R¹⁴ and R¹⁵ are each independently substituted with one or more lower substituent groups.

In embodiments, R¹⁴ and R¹⁵ are each independently substituted (e.g. with a substituent group, a size-limited substituent group or a lower substituent group) C₂-C₃₀ alkyl. In embodiments, R¹⁴ and R¹⁵ are each independently unsubstituted C₂-C₃₀ alkyl. In embodiments, R¹⁴ and R¹⁵ are each independently substituted (e.g. with a substituent group, a size-limited substituent group or a lower substituent group) 2- to 30-membered heteroalkyl. In embodiments, R¹⁴ and R¹⁵ are each independently unsubstituted 2- to 30-membered heteroalkyl.

In embodiments, R¹⁴ and R¹⁵ are each independently substituted or unsubstituted C₂-C₃₀ alkyl. In embodiments, R¹⁴ and R¹⁵ are each independently substituted (e.g. with a substituent group, a size-limited substituent group or a lower substituent group) or unsubstituted C₂-C₃₀ alkyl.

In embodiments, each R²¹⁰, R²¹¹, R³¹⁰, and R³¹¹ is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2- to 12-membered heteroalkyl. In embodiments, each R²¹⁰, R²¹¹, R³¹⁰, and R³¹¹ is independently substituted with one or more substituent groups. In embodiments, each R²¹⁰, R²¹¹, R³¹⁰, and R³¹¹ is independently substituted with one or more size-limited substituent groups. In embodiments, each R²¹, R²¹¹, R³¹⁰, and R³¹¹ is independently substituted with one or more lower substituent groups.

In embodiments, each R²¹⁰, R²¹¹, R³¹⁰, and R³¹¹ is independently H. In embodiments, each R²¹⁰, R²¹¹, R³¹⁰, and R³¹¹ is independently substituted (e.g. with a substituent group, a size-limited substituent group or a lower substituent group) C₁-C₁₂ alkyl. In embodiments, each R²¹⁰, R²¹¹, R³¹⁰, and R³¹¹ is independently unsubstituted C₁-C₁₂ alkyl. In embodiments, each R²¹, R²¹¹, R³¹⁰, and R³¹¹ is independently substituted (e.g. with a substituent group, a size-limited substituent group or a lower substituent group) 2- to 12-membered heteroalkyl. In embodiments, each R²¹⁰, R²¹¹, R³¹⁰, and R³¹¹ is independently unsubstituted 2- to 12-membered heteroalkyl.

In embodiments, each n is independently an integer from 0 to 8. In embodiments, each n is independently an integer from 0 to 4. In embodiments, each n is independently 8. In embodiments, each n is independently 7. In embodiments, each n is independently 6. In embodiments, each n is independently 5. In embodiments, each n is independently 4. In embodiments, each n is independently 3. In embodiments, each n is independently 2. In embodiments, each n is independently 1. In embodiments, each n is independently 0.

In embodiments, each s is an integer from 1 to 4. In embodiments, each s is 4. In embodiments, each s is 3. In embodiments, each s is 2. In embodiments, each s is 1.

In embodiments,
L¹¹ is

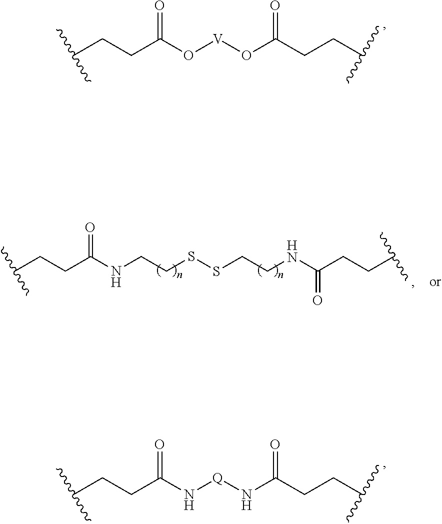

where Q is substituted or unsubstituted alkylene, V is substituted or unsubstituted alkylene and each n is independently an integer from 0 to 8.
B⁸, B⁹, B¹⁰, and B¹¹ are each independently substituted or unsubstituted alkylene.
L¹² is —O(C═O)— or —(C═O)O—. L¹³ is —O(C═O)— or —(C═O)O—. R¹² is H, —OR^{12A}, or —NR^{12A}.
R¹³ is H, —OR¹³A, or —NR^{13A}. R¹¹ and R¹⁵ are each independently substituted or unsubstituted C₂-C₃₀ alkyl. R^{12A} and R^{13A} are each independently H, substituted or unsubstituted C₁-C₂₀ alkyl.

In embodiments,
L¹¹ is

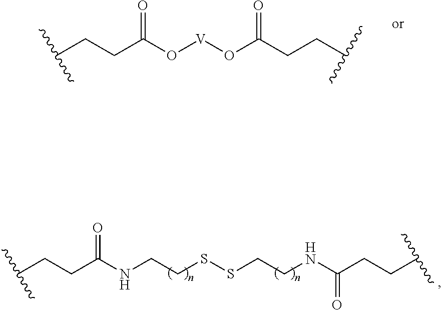

where V is substituted or unsubstituted alkylene and each n is independently an integer from 0 to 4. B⁸, B⁹, B¹⁰, and B¹¹ are each independently substituted or unsubstituted C₁-C₂₀ alkylene. L¹² is —O(C═O)— or —(C═O)O—. L¹³ is —O(C═O)— or —(C═O)O—. R′² is H or —OR¹²A. R¹³ is H or —OR^{13A}. R¹⁴ and R¹⁵ are each independently substituted or unsubstituted C₂-C₂₀ alkyl. R^{12A} and R^{13A} are each independently H, substituted or unsubstituted C₁-C₈ alkyl.

In embodiments,
L¹¹ is

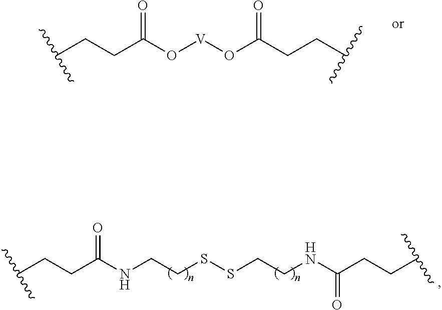

where V is substituted or unsubstituted alkylene and each n is independently an integer from 0 to 4.
B⁸, B⁹, B¹⁰, and B¹¹ are each independently substituted or unsubstituted C₁-C₈ alkylene.
L¹² is —O(C═O)— or —(C═O)O—. L¹³ is —O(C═O)— or —(C═O)O—. R′² is —OH, methoxy, or ethoxy.
R¹³ is —OH, methoxy, or ethoxy. R¹⁴ and R¹⁵ are each independently substituted or unsubstituted C₂-C₂₀ alkyl.

In embodiments, the cationic lipid of formula (III) is:

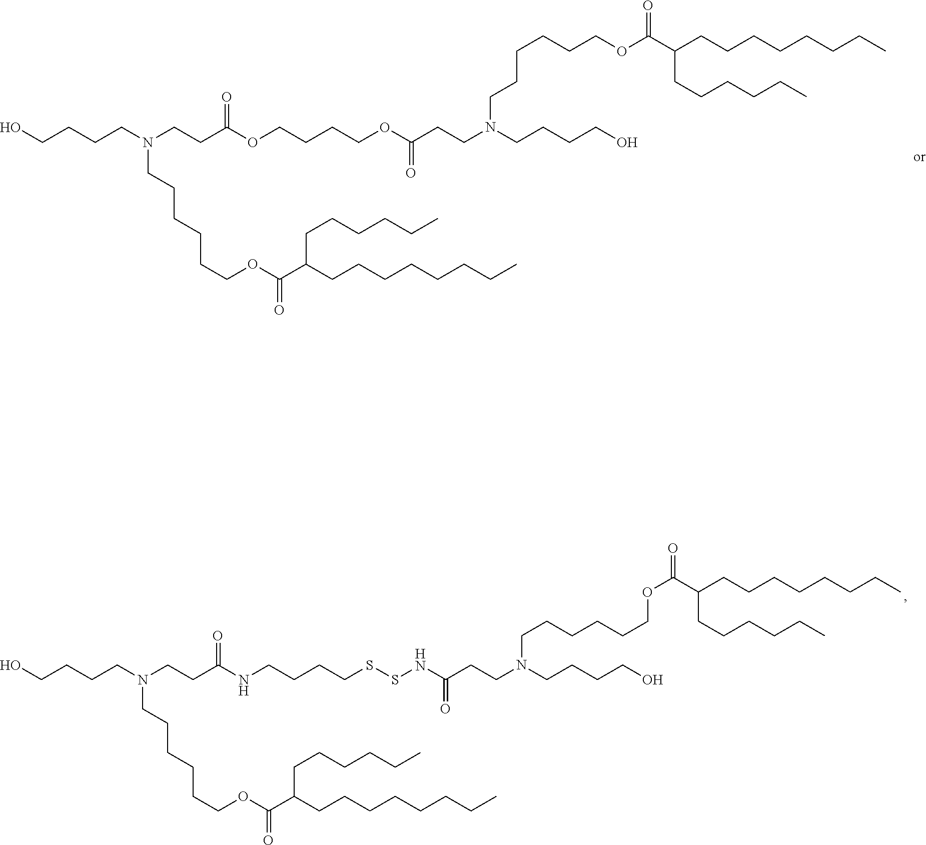 [SMILES: CCCCCCCCC(CCCCCC)C(=O)OCCCCCCN(CCCCO)CCC(=O)OCCCCOC(=O)CCN(CCCCO)CCCCCCOC(=O)C(CCCCCC)CCCCCCCC ; CCCCCCCCC(CCCCCC)C(=O)OCCCCCCN(CCCCO)CCC(=O)NCCCCSSNC(=O)CCN(CCCCO)CCCCCCOC(=O)C(CCCCCC)CCCCCCCC]

or a pharmaceutically acceptable salt thereof.

In an aspect, provided herein is cationic lipid of formula (IV):

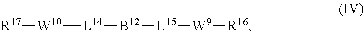

or a pharmaceutically acceptable salt, solvate, hydrate, stereoisomer, or prodrug thereof.
B¹² is —W⁷-L^{a3}-W⁸—.
W⁷ and W⁸ are each independently a bond, substituted or unsubstituted C₁-C₁₂ alkylene, or substituted or unsubstituted 2- to 12-membered heteroalkylene.
L^{a3} is a bond, —S—S—, —O—(CH₂O)m-,

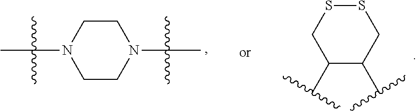 [SMILES: *N1CCN(*)CC1 ; *C1CSSCC1*]

W⁹ and W¹⁰ are each independently a bond, substituted or unsubstituted C₁-C₁₂ alkylene, substituted or unsubstituted 2- to 12-membered heteroalkylene, substituted or unsubstituted cycloalkylene, substituted or unsubstituted heterocycloalkylene, or any combination thereof.
L¹⁴ is a bond, —O(C═O)—, —(C═O)O—, —O(C═O)O—, —C(═O)—, —O—, —O(CR⁴¹⁰R⁴¹¹)ₛO—, —S—, —C(═O)S—, —SC(═O)—, —NR⁴¹⁰C(═O)—, —C(═O)NR⁴¹⁰—, —NR⁴¹⁰C(═O)NR⁴¹¹—, —NR⁴¹⁰C(═S)—, —C(═S)NR⁴¹⁰—, —NR⁴¹⁰C(═S)NR⁴¹¹—, —OC(═O)NR⁴¹⁰—, —NR⁴¹⁰C(═O)O—, —SC(═O)NR⁴¹⁰— or —NR⁴¹⁰C(═O)S—.
L¹⁵ is a bond, —O(C═O)—, —(C═O)O—, —O(C═O)O—, —C(═O)—, —O—, —O(CR^{51O}R⁵¹¹)ₛO—, —S—, —C(═O)S—, —SC(═O)—, —NR⁵¹⁰C(═O)—, —C(═O)NR⁵¹⁰—, —NR⁵¹⁰C(═O)NR⁵¹¹—, —NR⁵¹⁰C(═S)—, —C(═S)NR⁵¹⁰—, —NR⁵¹⁰C(═S)NR⁵¹¹—, —OC(═O)NR⁵¹⁰—, —NR⁵¹⁰C(═O)O—, —SC(═O)NR⁵¹⁰— or —NR⁵¹⁰C(═O)S—.
R¹⁶ and R¹⁷ are each independently

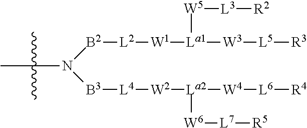

a fragment of cationic lipid of formula (I),

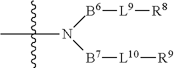

fragment of cationic lipid of formula (II),

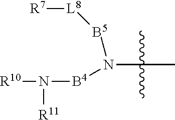

fragment of cationic lipid of formula (II),

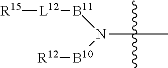

a fragment of cationic lipid of formula (III), or

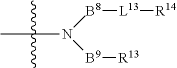

a fragment of cationic lipid of formula (ITI).
each R⁴¹⁰, R⁴¹¹, R⁵¹⁰, and R⁵¹¹ is independently H, substituted or unsubstituted C₁-C₁₂ alkyl, or substituted or unsubstituted 2- to 12-membered heteroalkyl.
each m is independently an integer from 0 to 8, and
each s is independently an integer from 1 to 4.

In embodiments, W⁷ and W⁸ are each independently a bond, substituted (e.g. with a substituent group, a size-limited substituent group or a lower substituent group) or unsubstituted C₁-C₁₂ alkylene, or substituted (e.g. with a substituent group, a size-limited substituent group or a lower substituent group) or unsubstituted 2- to 12-membered heteroalkylene. In embodiments, W⁷ and W⁸ are each independently substituted with one or more substituent groups. In embodiments, W⁷ and W⁸ are each independently substituted with one or more size-limited substituent groups. In embodiments, W⁷ and W⁸ are each independently substituted with one or more lower substituent groups.

In embodiments, W⁷ and W⁸ are each independently a bond. In embodiments, W⁷ and W⁸ are each independently substituted (e.g. with a substituent group, a size-limited substituent group or a lower substituent group) CL-C₁₂ alkylene. In embodiments, W⁷ and W⁸ are each independently unsubstituted C₁-C₁₂ alkylene. In embodiments, W⁷ and W⁸ are each independently substituted (e.g. with a substituent group, a size-limited substituent group or a lower substituent group) 2- to 12-membered heteroalkylene. In embodiments, W⁷ and W⁸ are each independently unsubstituted 2- to 12-membered heteroalkylene.

In embodiments, W⁷ and W⁸ are each independently a bond or substituted or unsubstituted C₁-C₁₂ alkylene. W⁷ and W⁸ are each independently a bond or substituted (e.g. with a substituent group, a size-limited substituent group or a lower substituent group) or unsubstituted C₁-C₁₂ alkylene

In embodiments, W⁷ and W⁸ are each independently a bond or unsubstituted C₁-C₁₂ alkylene.

In embodiments, W⁷ and W⁸ are each independently a bond or unsubstituted C₁-C₈ alkylene. In embodiments, W⁷ and W⁸ are each independently unsubstituted C₁-C₈ alkylene.

In embodiments, L^{a3} is a bond, —S—S—,

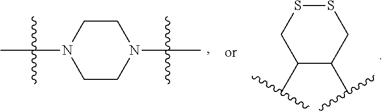 [SMILES: *N1CCN(*)CC1 ; *C1CSSCC1*]

In embodiments, L^{a3} is a bond. In embodiments, L^{a3} is —S—S—. In embodiments, L^{a3} is

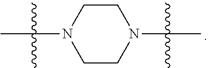 [SMILES: *N1CCN(*)CC1]

In embodiments, L^{a3} is

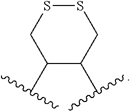 [SMILES: *C1CSSCC1*]

In embodiments, W⁹ and W¹⁰ are each independently a bond, substituted (e.g. with a substituent group, a size-limited substituent group or a lower substituent group) or unsubstituted C₁-C₁₂ alkylene, substituted (e.g. with a substituent group, a size-limited substituent group or a lower substituent group) or unsubstituted 2- to 12-membered heteroalkylene, substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene), substituted (e.g. with a substituent group, a size-limited substituent group or a lower substituent group) or unsubstituted heterocycloalkylene(e.g., 3- to 8-membered heterocycloalkylene, 3- to 6-membered heterocycloalkylene, or 5- to 6-membered heterocycloalkylene), or any combination thereof. In embodiments, W⁹ and W¹⁰ are each independently substituted with one or more substituent groups. In embodiments, W⁹ and W¹⁰ are each independently substituted with one or more size-limited substituent groups. In embodiments, W⁹ and W¹⁰ are each independently substituted with one or more lower substituent groups.

In embodiments, W⁹ and W¹⁰ are each independently a bond. In embodiments, W⁹ and W¹⁰ are each independently substituted (e.g. with a substituent group, a size-limited substituent group or a lower substituent group) C₁-C₁₂ alkylene. In embodiments, W⁹ and W¹⁰ are each independently unsubstituted C₁-C₁₂ alkylene. In embodiments, W⁹ and W¹⁰ are each independently substituted (e.g. with a substituent group, a size-limited substituent group or a lower substituent group) 2- to 12-membered heteroalkylene. In embodiments, W⁹ and W¹⁰ are each independently unsubstituted 2- to 12-membered heteroalkylene. In embodiments, W⁹ and W¹⁰ are each independently substituted (e.g. with a substituent group, a size-limited substituent group or a lower substituent group) cycloalkylene (e.g., C₃-C₈ cycloalkylene, C₃-C₆ cycloalkylene, or C₅-C₆ cycloalkylene). In embodiments, W⁹ and W¹⁰ are each independently unsubstituted cycloalkylene (e.g., C₃-C₈ cycloalkylene, C₃-C₆ cycloalkylene, or C₅-C₆ cycloalkylene). In embodiments, W⁹ and W¹⁰ are each independently substituted (e.g. with a substituent group, a size-limited substituent group or a lower substituent group) heterocycloalkylene(e.g., 3- to 8-membered heterocycloalkylene, 3- to 6-membered heterocycloalkylene, or 5- to 6-membered heterocycloalkylene). In embodiments, W⁹ and W¹⁰ are each independently unsubstituted heterocycloalkylene(e.g., 3- to 8-membered heterocycloalkylene, 3- to 6-membered heterocycloalkylene, or 5- to 6-membered heterocycloalkylene).

In embodiments, W⁹ and W¹⁰ are each independently a bond or substituted or unsubstituted C₁-C₁₂ alkylene. In embodiments, W⁹ and W¹⁰ are each independently a bond or substituted (e.g. with a substituent group, a size-limited substituent group or a lower substituent group) or unsubstituted C₁-C₁₂ alkylene. In embodiments, W⁹ and W¹⁰ are each independently substituted (e.g. with a substituent group, a size-limited substituent group or a lower substituent group) C₁-C₁₂ alkylene. In embodiments, W⁹ and W¹⁰ are each independently unsubstituted C₁-C₁₂ alkylene.

In embodiments, W⁹ and W¹⁰ are each independently a bond or unsubstituted C₁-C₈ alkylene. In embodiments, W⁹ and W¹⁰ are each independently unsubstituted C₁-C₈ alkylene.

In embodiments, L¹⁴ is-O(C═O)—, —(C═O)O—, —C(═O)—, —NR⁴¹⁰C(═O)—, —C(═O)NR⁴¹⁰—, —NR⁴¹⁰C(═S)—, —C(═S)NR⁴¹⁰—, —OC(═O)NR⁴¹⁰—, or —NR⁴¹⁰C(═O)O—. In embodiments, L¹⁴ is —O(C═O)—, —(C═O)O—, —NR⁴¹⁰C(═S)—, —C(═S)NR⁴¹⁰—, —OC(═O)NR⁴¹⁰—, or —NR⁴¹⁰C(═O)O—.

In embodiments, L¹⁴ is —O(C═O)—. In embodiments, L¹⁴ is —(C═O)O—. In embodiments, L¹⁴ is-C(═O)—. In embodiments, L¹⁴ is —NR⁴¹⁰C(═O)—. In embodiments, L¹⁴ is —C(═O)NR⁴¹⁰—. In embodiments, L¹⁴ is —NR⁴¹⁰C(═S)—. In embodiments, L¹⁴ is —C(═S)NR⁴¹⁰—. In embodiments, L¹⁴ is —OC(═O)NR⁴¹⁰—. In embodiments, L¹⁴ is —NR⁴¹⁰C(═O)O—.

In embodiments, L¹⁵ is-O(C═O)—, —(C═O)O—, —C(═O)—, —NR⁵¹⁰C(═O)—, —C(═O)NR⁵¹⁰—, —NR⁵¹⁰C(═S)—, —C(═S)NR⁵¹⁰—, —OC(═O)NR⁵¹⁰—, or —NR⁵¹⁰C(═O)O—. In embodiments, L¹⁵ is —O(C═O)—, —(C═O)O—, —NR⁵¹⁰C(═S)—, —C(═S)NR⁵¹⁰—, —OC(═O)NR⁵¹⁰—, or —NR⁵¹C(═O)O—.

In embodiments, L¹⁵ is —O(C═O)—. In embodiments, L¹⁵ is —(C═O)O—. In embodiments, L¹⁵ is-C(═O)—. In embodiments, L¹⁵ is —NR⁵¹⁰C(═O)—. In embodiments, L¹⁵ is —C(═O)NR⁵¹⁰—. In embodiments, L¹⁵ is —NR⁵¹⁰C(═S)—. In embodiments, L¹⁵ is —C(═S)NR⁵¹⁰—. In embodiments, L¹⁵ is —OC(═O)NR⁵¹—. In embodiments, L⁵ is —NR⁵¹⁰C(═O)O—.

In embodiments, each R⁴¹⁰, R⁴¹¹, R⁵¹⁰, and R⁵¹¹ is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2- to 12-membered heteroalkyl. In embodiments, each R⁴¹⁰, R⁴¹¹, R⁵¹, and R⁵¹¹ is independently substituted with one or more substituent groups. In embodiments, each R⁴¹⁰, R⁴¹¹, R⁵¹⁰, and R⁵¹¹ is independently substituted with one or more size-limited substituent groups. In embodiments, each R⁴¹⁰, R⁴¹¹, R⁵¹⁰, and R⁵¹¹ is independently substituted with one or more lower substituent groups.

In embodiments, each R⁴¹⁰, R⁴¹¹, R⁵¹⁰, and R⁵¹¹ is independently H. In embodiments, each R⁴¹⁰, R⁴¹¹, R⁵¹⁰, and R⁵¹¹ is independently substituted (e.g. with a substituent group, a size-limited substituent group or a lower substituent group) C₁-C₁₂ alkyl. In embodiments, each R⁴¹⁰, R⁴¹¹, R⁵¹⁰, and R⁵¹¹ is independently unsubstituted C₁-C₁₂ alkyl. In embodiments, each R⁴¹⁰, R⁴¹¹, R⁵¹⁰, and R⁵¹¹ is independently substituted (e.g. with a substituent group, a size-limited substituent group or a lower substituent group) 2- to 12-membered heteroalkyl. In embodiments, each R⁴¹⁰, R⁴¹¹, R⁵¹⁰, and R⁵¹¹ is independently unsubstituted 2- to 12-membered heteroalkyl.

In embodiments, each R⁴¹⁰ and R⁵¹⁰ is independently H or substituted or unsubstituted C₁-C₁₂ alkyl. In embodiments, each R⁴¹⁰ and R¹⁰ is independently H or substituted (e.g. with a substituent group, a size-limited substituent group or a lower substituent group) or unsubstituted C₁-C₁₂ alkyl.

In embodiments, each R⁴¹⁰ and R⁵¹⁰ is independently H or unsubstituted C₁-C₈alkyl. In embodiments, each R⁴¹⁰ and R⁵¹⁰ is independently unsubstituted C₁-C₈ alkyl.

In embodiments, each R⁴¹⁰ and R⁵¹⁰ is independently H, methyl, ethyl, propyl, butyl, pentyl, hexyl, heptyl, or octyl. In embodiments, each R⁴¹⁰ and R⁵¹⁰ is independently H or methyl. In embodiments, each R⁴¹⁰ and R⁵¹⁰ is independently H. In embodiments, each R⁴¹⁰ and R⁵¹ is independently methyl.

In embodiments, R¹⁶ and R¹⁷ are each independently

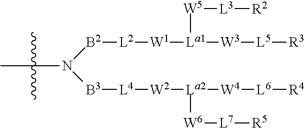

a fragment of cationic lipid of formula (I),

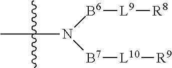

a fragment of cationic lipid of formula (II),

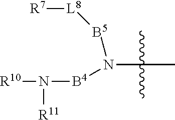

fragment of cationic lipid of formula (II),

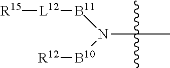

a fragment of cationic lipid of formula (III), or

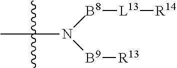

a fragment of cationic lipid of formula (III), where B², B³, B⁴, B⁵, B⁶, B⁷, B⁸, B⁹, B¹⁰, B¹¹, L⁹, L², L³, L⁴, L⁵, L⁶, L⁷, L⁸, L⁹, L¹⁰, L¹², L¹³, R², R³, R⁴, R⁵, R⁷, R⁸, R⁹, R¹⁰, R¹¹, R¹², R¹³, R¹⁴, and R¹⁵ are as described herein including embodiments.

In embodiments, R¹⁶ and R¹⁷ are each independently

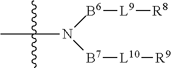

a fragment of cationic lipid of formula (II), where B⁶, B⁷, L⁹, L¹⁰, R⁸, and R⁹ are as described herein including embodiments.

In embodiments, R¹⁶ and R¹⁷ are each independently

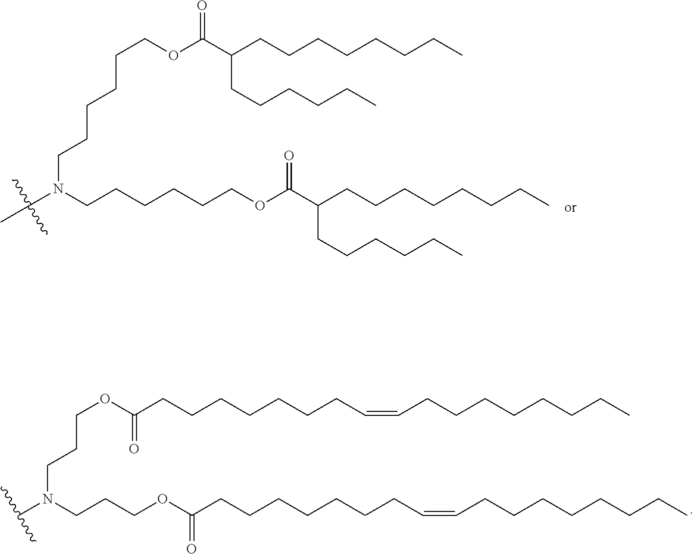 [SMILES: *N(CCCCCCOC(=O)C(CCCCCC)CCCCCCCC)CCCCCCOC(=O)C(CCCCCC)CCCCCCCC ; *N(CCCOC(=O)CCCCCCC/C=C\CCCCCCCC)CCCOC(=O)CCCCCCC/C=C\CCCCCCCC]

In embodiments, R¹⁶ and R¹⁷ are each independently

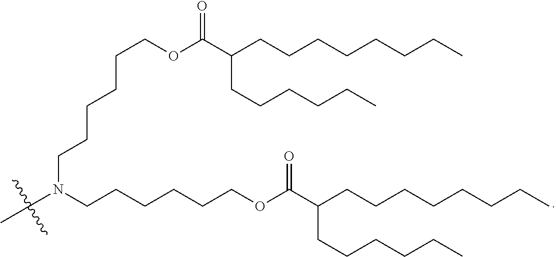 [SMILES: *N(CCCCCCOC(=O)C(CCCCCC)CCCCCCCC)CCCCCCOC(=O)C(CCCCCC)CCCCCCCC]

In embodiments, R¹⁶ and R¹⁷ are each independently

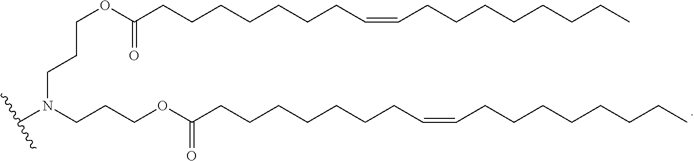 [SMILES: *N(CCCOC(=O)CCCCCCC/C=C\CCCCCCCC)CCCOC(=O)CCCCCCC/C=C\CCCCCCCC]

In embodiments, each m is independently an integer from 0 to 8. In embodiments, each m is independently 8. In embodiments, each m is independently 7. In embodiments, each m is independently 6. In embodiments, each m is independently 5. In embodiments, each m is independently 4. In embodiments, each m is independently 3. In embodiments, each m is independently 2. In embodiments, each m is independently 1. In embodiments, each m is independently 0.

In embodiments, each s is an integer from 1 to 4. In embodiments, each s is 4. In embodiments, each s is 3. In embodiments, each s is 2. In embodiments, each s is 1.

In embodiments, the cationic lipid of formula (IV) is:

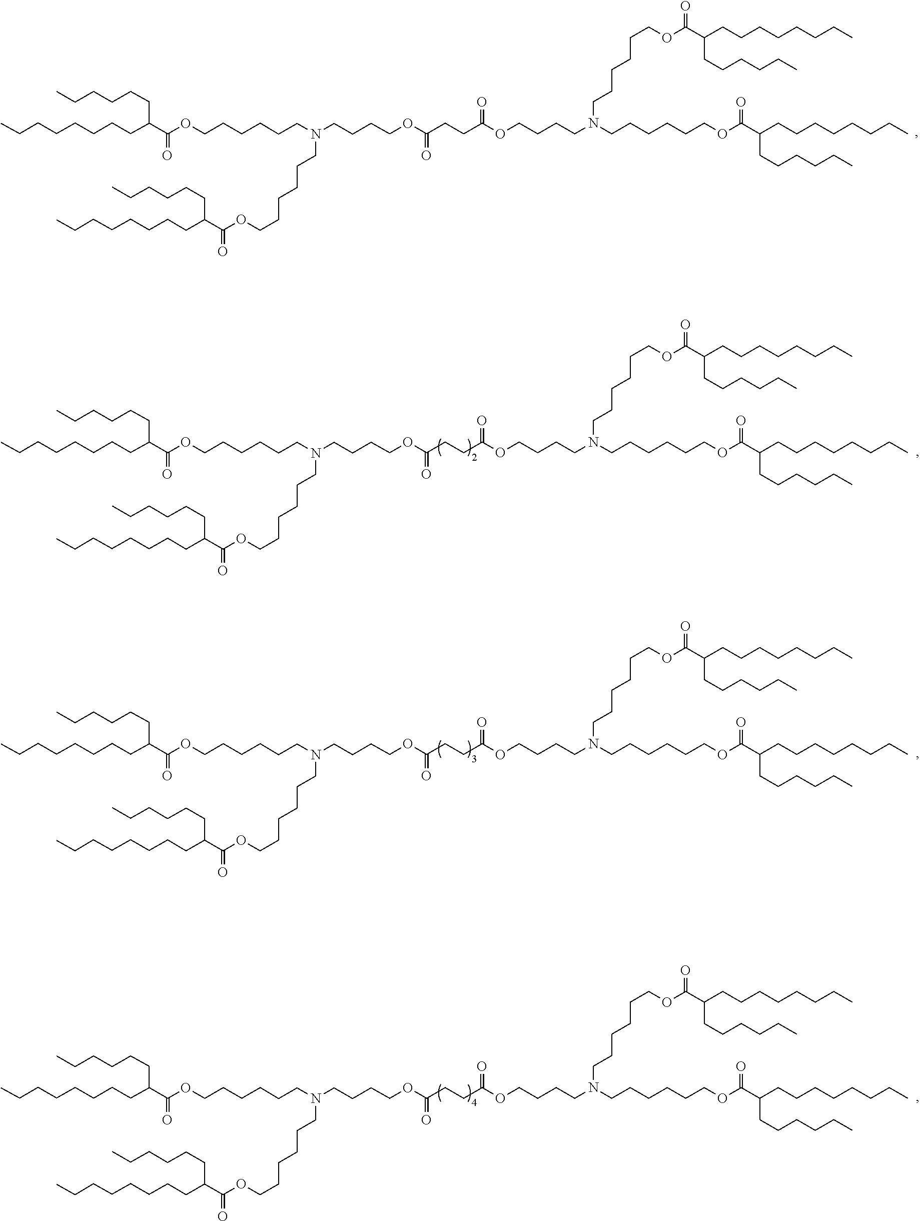

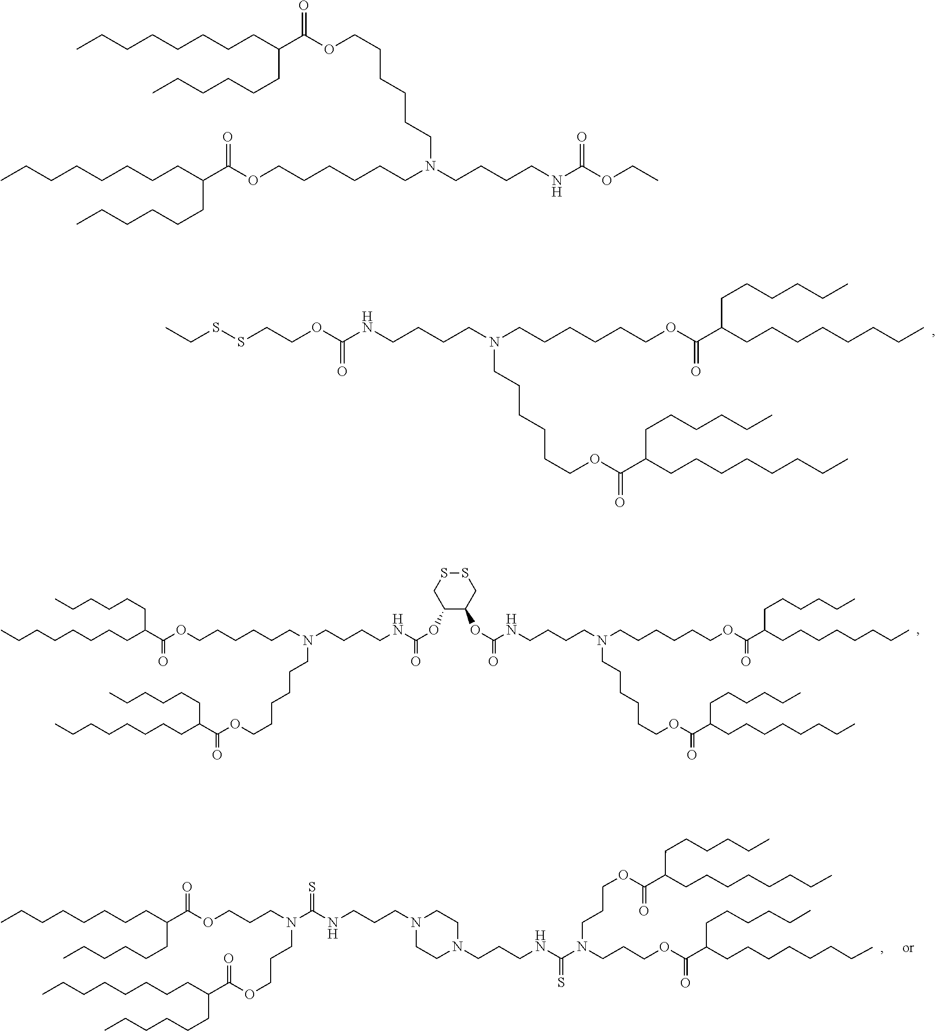

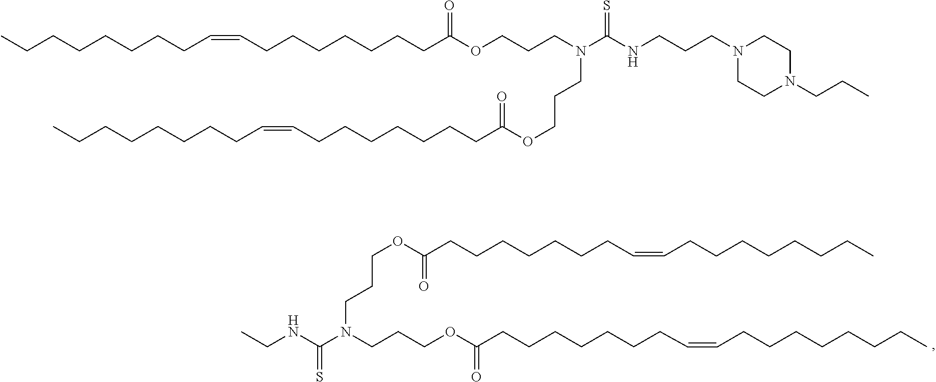

or a pharmaceutically acceptable salt thereof.

### Lipid Nanoparticles

In an aspect, provided herein are lipid nanoparticles comprising one or more of the ionizable cationic lipids or salts thereof described herein. In embodiments, the lipid nanoparticles described herein further include one or more non-cationic lipids. In embodiments, the lipid nanoparticles described herein further include one or more conjugated lipids capable of reducing or inhibiting particle aggregation. In other embodiments, the lipid nanoparticles described herein further include one or more therapeutic agents such as nucleic acids (e.g., mRNA).

In embodiments, lipid nanoparticles comprising one or more ionizable cationic lipids described herein are used to encapsulate nucleic acids (e.g., mRNA) within the lipid nanoparticles.

In embodiments, the lipid nanoparticles include a therapeutic agent such as nucleic acid (e.g., mRNA), a cationic lipid (one or more ionizable cationic lipids of formula I-IV or salts thereof, as described herein, or cationic lipids known in the art), a non-cationic lipid (e.g., mixtures of one or more phospholipids and cholesterol), and a conjugated lipid that inhibits aggregation of particles (e.g., one or more PEG-lipid conjugates).

In embodiments, non-cationic lipids that can be used in the lipid nanoparticles described herein include, without limitation, neutral, zwitterionic or anionic lipids, for example: phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyolphosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

In embodiments, non-cationic lipids may be sterols such as cholesterol and derivatives thereof. Non-limiting examples of cholesterol derivatives include polar analogues such as 5a-cholestanol, 5p-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5u-cholestanone, 5p-cholestanone, and cholesteryl decanoate; and mixtures thereof. In embodiments, the cholesterol derivative is a polar analogue such as cholesteryl-(4′-hydroxy)-butyl ether.

In embodiments, the non-cationic lipids included in the lipid nanoparticles include a mixture of one or more phospholipids and cholesterol or a derivative thereof.

In embodiments, non-cationic lipids suitable for use in the lipid nanoparticles include stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In embodiments, lipid conjugates that can be used in the lipid nanoparticles described herein include, without limitation, PEG-lipid conjugates, POZ-lipid conjugates, ATTA-lipid conjugates, cationic-polymer-lipid conjugates (CPLs), and mixtures thereof. In embodiments, the nanoparticles comprise PEG-lipid conjugate.

In embodiments, lipid conjugates that can be used in the lipid nanoparticles described herein include, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to ceramides, mPEG2000-1,2-di-O-alkyl-sn3-carbomoylglyceride (PEG-C-DOMG), 1-[8′-(1,2-dimyristoyl-3-propanoxy)-carboxamido-3′,6′-dioxaoctanyl]carbamoyl-co-methyl-poly(ethylene glycol) (2 KPEG-DMG), 1,2-Dimyristoyl-rac-glycero-3-methylpolyoxyethylene (DMG-PEG), PEG conjugated to cholesterol or a derivative thereof, and mixtures thereof.

In embodiments, lipid nanoparticles described herein are useful for the introduction of therapeutic agents such as nucleic acids (e.g., mRNA) into cells.

In an aspect, provided herein is a method for the in vivo delivery of a therapeutic agent comprising administering the lipid nanoparticle, composed of the ionizable cationic lipids of formula I-IV as described herein, to a mammal.

In embodiments, the lipid nanoparticles described herein can be administered either alone or in a mixture with a pharmaceutically acceptable carrier. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like.

The pharmaceutically acceptable carrier is usually added following lipid nanoparticle formation. Thus, after the lipid nanoparticle is formed, the nanoparticle can be diluted into pharmaceutically acceptable carriers such as normal buffered saline.

For in vivo administration, administration can be in any manner known in the art, e.g., by injection, oral administration, inhalation (e.g., intransal or intratracheal), transdermal application, or rectal administration.

In embodiments, the pharmaceutical compositions can be administered parenterally, i.e., intraarticularly, intravenously, intraperitoneally, subcutaneously, or intramuscularly. In embodiments, the pharmaceutical compositions are administered intravenously or intraperitoneally by a bolus injection.

In an aspect, provided herein is a method for preventing or treating a disease in a mammal in need thereof by administering to the mammal a therapeutically effective amount of a lipid nanoparticle composed of the ionizable cationic lipids of formula I-IV as described herein. In embodiments, provided herein is a method for preventing a disease in a mammal by administering to the mammal a therapeutically effective amount of a lipid nanoparticle composed of the ionizable cationic lipids of formula I-IV as described herein. In embodiments, provided herein is a method for treating a disease in a mammal, in need thereof, by administering to the mammal a therapeutically effective amount of a lipid nanoparticle composed of the ionizable cationic lipids of formula I-IV as described herein.

In embodiments the mammal is a dog, a cat or a human. In embodiments, the mammal is a dog. In embodiments, the mammal is a cat. In embodiments, the mammal is a human.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry.

The following examples are meant to be illustrative and can be used to further understand embodiments of the present disclosure and should not be construed as limiting the scope of the present teachings in any way.

The chemical reactions described in the Examples can be readily adapted to prepare a number of other compounds of the present disclosure, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by utilizing other suitable reagents known in the art other than those described, or by making routing modifications of reaction conditions, reagents, and starting materials. Alternatively, other reactions disclosed herein or known in the art will be recognized as having applicability for preparing other compounds of the present disclosure.

### DEFINITIONS OF ABBREVIATIONS USED

AcN Acetonitrile
AcOH Acetic acid
aq. Aqueous
Boc tert-butyloxycarbonyl
CSA Camphorsulfonic acid
CSCl₂ Thiophosgene
DCM Dichloromethane
DMF N,N-Dimethylformamide
DMAP Dimethylaminopyridine
EDC 1-Ethyl-3-(3-dimethylaminopropyl)carbodiimide
Et Ethyl
Eq Equivalents
g Gram(s)
hr Hour (hours)
HCl hydrochloric acid
HPLC High-performance liquid chromatography
LC/MS Liquid chromatography-mass spectrometry
Me Methyl
mg Milligrams
MeOH Methanol
mL Milliliter(s)
μL/μL Microliter(s)
mol Moles
mmol Millimoles
μmol/μmol Micromoles
MS Mass spectrometry
NaBH(OAc)₃ Sodium triacetoxyborohydride
PCC Pyridinium chlorochromate
r.t. Room temperature
t-Bu tert-Butyl
TEA or Et₃N Triethylamine
TFA Trifluoracetic acid
THF Tetrahydrofuran
TLC Thin Layer Chromatography SYNTHETIC EXAMPLES

### Example S1: Synthesis of Compound KT-001 (a Compound of Formula (I))

Compound KT-001 was prepared as shown in Scheme 1 below.

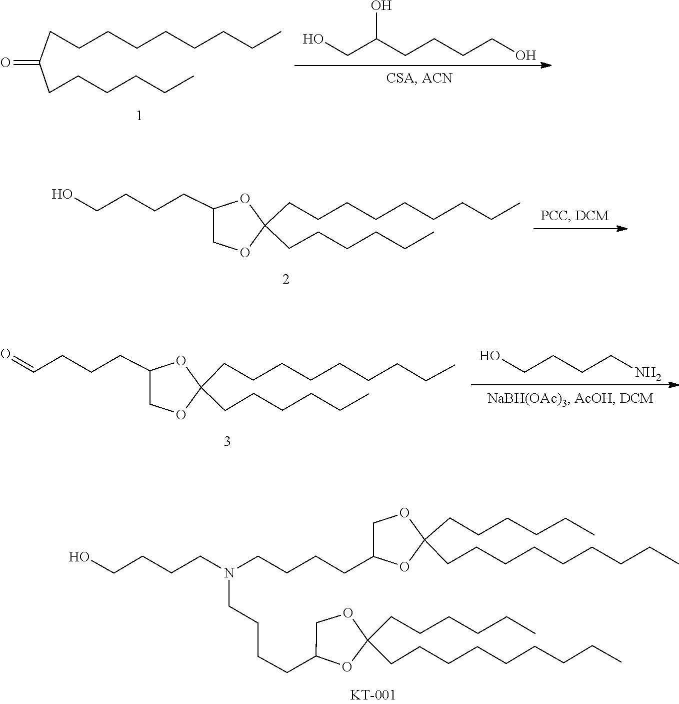

Compound 2. To a mixture of compound 1 (1.00 g, 4.16 mmol) and 1,2,6-hexanetriol (1.12 g, 8.32 mmol) in anhydrous acetonitrile (20 mL) was added camphorsulfonic acid (CSA) (290 mg, 1.25 mmol). The resulting solution was refluxed for 16 hr until TLC indicated completion of reaction. Compound 2 (650 mg) was obtained by silica gel chromatography.

Compound 3. Pyridinium chlorochromate (PCC) (785 mg, 3.64 mmol) was added to a solution of compound 2 (650 mg, 1.82 mmol) in dichloromethane (30 mL). The reaction was stirred at room temperature for 2 hr until TLC indicated completion of reaction. Compound 3 (320 mg) was obtained by silica gel chromatography.

Compound KT-001. Acetic acid (4 μL, 0.068 mmol) was added to a mixture of compound 3 (300 mg, 0.846 mmol) and 4-amino-1-butanol (30 mg, 0.338 mmol) in dichloromethane (5 mL). The resulting mixture was stirred at r.t. for 15 min followed by addition of sodium triacetoxyborohydride (NaBH(OAc)₃) (215 mg, 1.01 mmol) and stirred at r.t. for another 4 h. Compound KT-001 (165 mg) was obtained by silica gel chromatography. ¹HNMR (500 MHz, CDCl₃): δ=0.85-0.91 (m, 12H), 1.20-1.44 (m, 48H), 1.46-1.72 (m, 20H), 2.31-2.61 (br, 6H), 3.40-3.47 (m, 2H), 3.52-3.60 (m, 2H), 3.99-4.07 (m, 4H).

### Example S2: Synthesis of Compound TU-001 (a Compound of Formula (11))

Compound TU-001 was prepared as shown in Scheme 2 below.

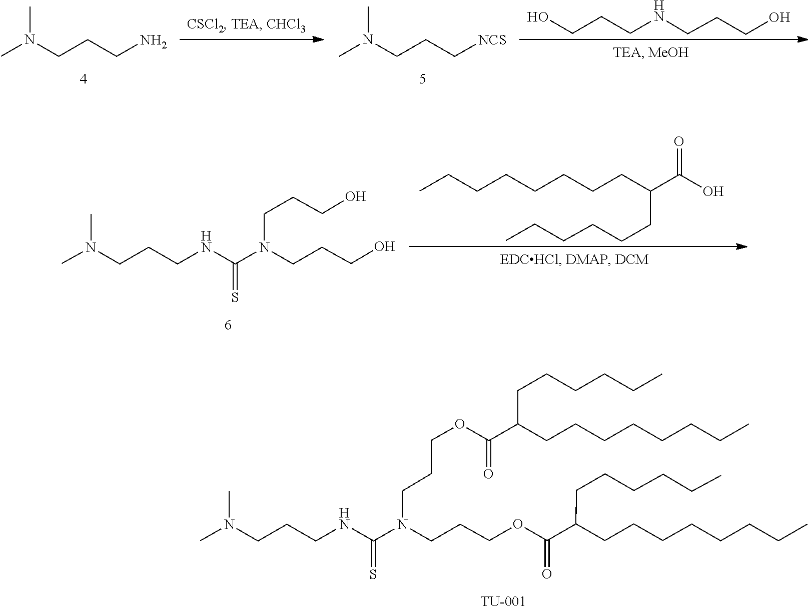

A solution of compound 4 (510 mg, 5.0 mmol) in DCM (10 mL) was cooled to 0° C. Thiophosgene (CSCl₂, 862 mg, 575 μL, 7.5 mmol) and TEA (1.4 mL, 10.0 mmol) were added to the solution. The resulting mixture was stirred at r.t. for 16 hr and then washed with sat. NaHCO₃. Compound 5 (720 mg) was used in the next step as a crude product.

TEA (1 mL) was added to the mixture of 5 (720 mg, 5.0 mmol) and bis(2-hydroxypropyl)amine (400 mg, 3.0 mmol) in MeOH (10 mL). The resulting mixture was stirred at r.t. for 16 hr until TLC indicated completion of reaction. Compound 6 (540 mg) was obtained by silica gel chromatography.

EDC·HCl (221 mg, 1.15 mmol) and DMAP (141 mg, 1.15 mmol) were added to the mixture of compound 6 (80 mg, 0.29 mmol) and 2-hexyldecanoic acid (296 mg, 338 μL, 1.15 mmol) in DCM (5 mL). The resulting mixture was stirred at r.t. for 16 hr until TLC indicated completion of reaction. Compound TU-001 (171 mg) was obtained by silica gel chromatography.

¹HNMR (500 MHz, CDCl₃): δ=0.88 (t, 12H, J=6.5 Hz), 1.15-1.65 (m, 54H), 1.93-2.05 (m, 6H), 2.26-2.40 (m, 4H), 3.62-3.81 (m, 6H), 4.13 (t, 4H, J=6.5 Hz).

### Example S3: Synthesis of Compound TU-002 (a Compound of Formula (II))

Compound TU-002 was prepared as shown in Scheme 3 below.

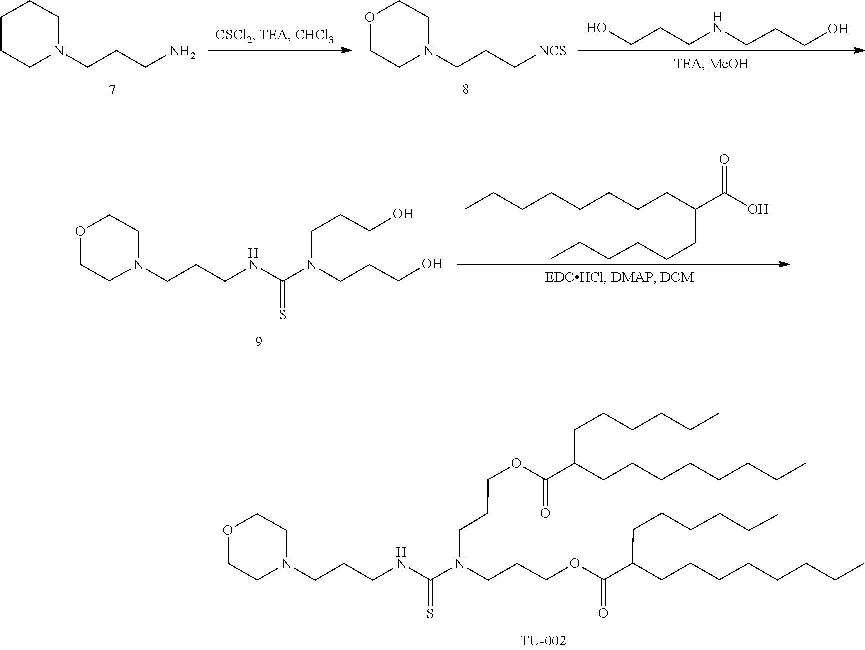

The solution of compound 7 (720 mg, 5.0 mmol) in DCM (10 mL) was cooled to 0° C. Thiophosgene (CSCl₂, 862 mg, 575 μL, 7.5 mmol) and TEA (1.4 mL, 10.0 mmol) were added to the solution. The resulting mixture was stirred at r.t. for 16 hr and then washed with sat. NaHCO₃·Compound 8 (930 mg) was used for next step as a crude product.

TEA (1 mL) was added to the mixture of compound 8 (930 mg, 5.0 mmol) and bis(2-hydroxypropyl)amine (400 mg, 3.0 mmol) in MeOH (10 mL). The resulting mixture was stirred at r.t. for 16 hr until TLC indicated completion of reaction. Compound 9 (630 mg) was obtained by silica gel chromatography.

EDC·HCl (192 mg, 1.00 mmol) and DMAP (122 mg, 1.00 mmol) were added to the mixture of compound 9 (80 mg, 0.25 mmol) and 2-hexyldecanoic acid (256 mg, 293 μL, 1.00 mmol) in DCM (5 mL). The resulting mixture was stirred at r.t. for 16 hr until TLC indicated completion of reaction. Compound TU-002 (158 mg) was obtained by silica gel chromatography.

¹HNMR (500 MHz, CDCl₃): δ=0.88 (t, 12H, J=7.0 Hz), 1.18-1.33 (m, 40H), 1.40-1.50 (m, 4H), 1.52-1.65 (m, 8H), 1.95-2.06 (m, 4H), 2.27-2.37 (m, 2H), 2.42-2.68 (m, 4H), 3.54-3.90 (m, 10H), 4.13 (t, 4H, J=6.5 Hz).

### Example S4: Synthesis of Compound TU-003 (a Compound of Formula (II))

Compound TU-003 was prepared as shown in Scheme 4 below.

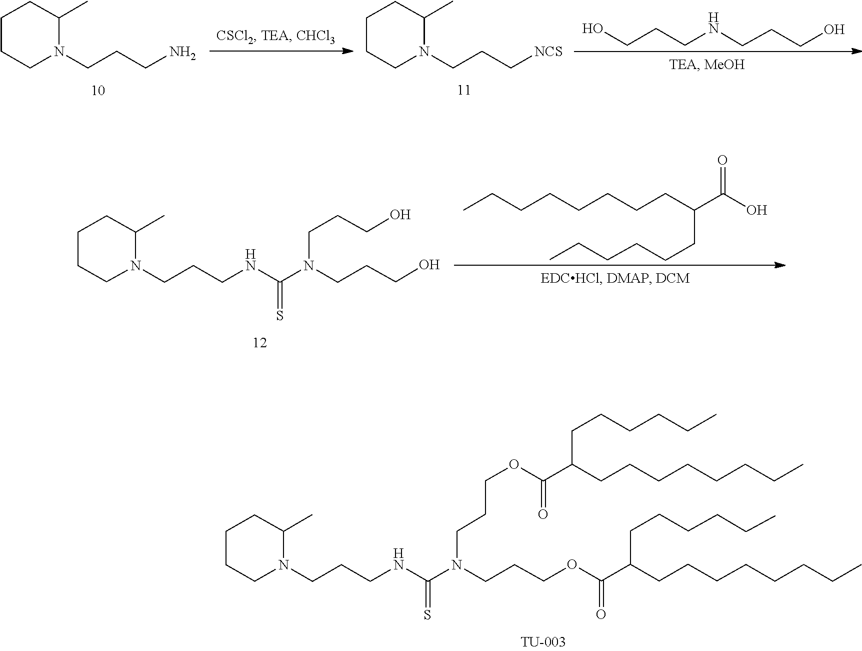

The solution of compound 10 (780 mg, 5.0 mmol) in DCM (10 mL) was cooled to 0° C. Thiophosgene (CSCl₂, 862 mg, 575 μL, 7.5 mmol) and TEA (1.4 mL, 10.0 mmol) were added to the solution. The resulting mixture was stirred at r.t. for 16 hr and then washed with sat. NaHCO₃. Compound 11 (990 mg) was used for next step as a crude product.

TEA (1 mL) was added to the mixture of compound 11 (990 mg, 5.0 mmol) and bis(2-hydroxypropyl)amine (400 mg, 3.0 mmol) in MeOH (10 mL). The resulting mixture was stirred at r.t. for 16 hr until TLC indicated completion of reaction. Compound 12 (670 mg) was obtained by silica gel chromatography.

EDC·HCl (174 mg, 0.91 mmol) and DMAP (111 mg, 0.91 mmol) were added to the mixture of compound 12 (75 mg, 0.23 mmol) and 2-hexyldecanoic acid (232 mg, 265 μL, 0.91 mmol) in DCM (5 mL). The resulting mixture was stirred at r.t. for 16 hr until TLC indicated completion of reaction. Compound TU-003 (133 mg) was obtained by silica gel chromatography.

¹HNMR (500 MHz, CDCl₃): δ=0.87 (t, 12H, J=7.0 Hz), 1.06 (d, 3H, J=6.5 Hz), 1.19-1.33 (m, 45H). 1.40-1.50 (m, 5H), 1.54-1.68 (m, 10H), 1.96-2.04 (m, 4H), 2.27-2.37 (m, 3H), 3.55-3.84 (m, 6H), 4.12 (t, 4H, J=6.5 Hz).

### Example S5: Synthesis of Compound TU-004 (a Compound of Formula (11))

Compound TU-004 was prepared as shown in Scheme 5 below.

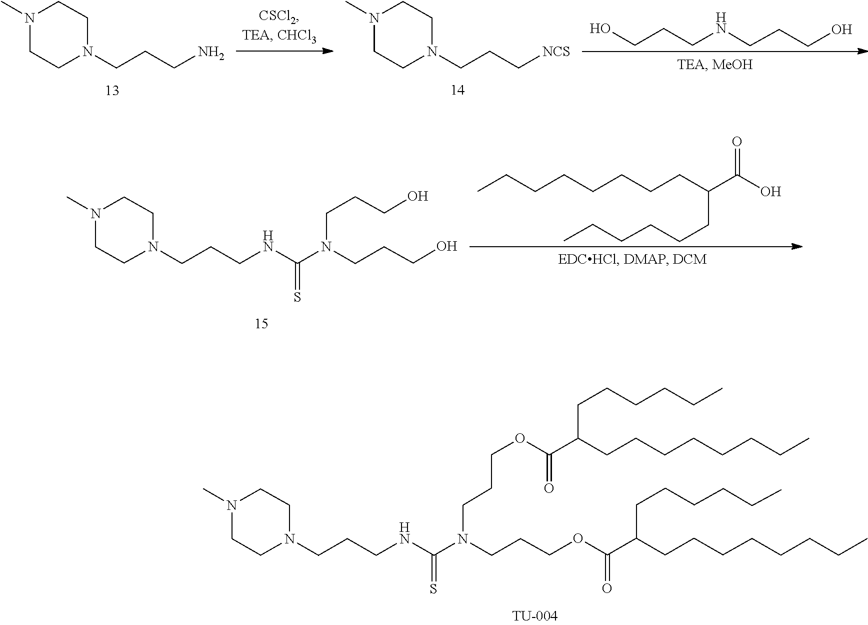

The solution of compound 13 (785 mg, 5.0 mmol) in DCM (10 mL) was cooled to 0° C. Thiophosgene (CSCl₂, 862 mg, 575 μL, 7.5 mmol) and TEA (1.4 mL, 10.0 mmol) were added to the solution. The resulting mixture was stirred at r.t. for 16 hr and then washed with sat. NaHCO₃·Compound 14 (995 mg) was used for next step as a crude product.

TEA (1 mL) was added to the mixture of compound 14 (995 mg, 5.0 mmol) and bis(2-hydroxypropyl)amine (400 mg, 3.0 mmol) in MeOH (10 mL). The resulting mixture was stirred at r.t. for 16 hr until TLC indicated completion of reaction. Compound 15 (630 mg) was obtained by silica gel chromatography.

EDC·HCl (173 mg, 0.90 mmol) and DMAP (110 mg, 0.90 mmol) were added to the mixture of compound 15 (75 mg, 0.23 mmol) and 2-hexyldecanoic acid (231 mg, 264 μL, 0.90 mmol) in DCM (5 mL). The resulting mixture was stirred at r.t. for 16 hr until TLC indicated completion of reaction. Compound TU-004 (127 mg) was obtained by silica gel chromatography.

¹HNMR (500 MHz, CDCl₃): δ=0.87 (t, 12H, J=7.0 Hz), 1.19-1.32 (m, 40H), 1.39-1.49 (m, 4H), 1.53-1.68 (m, 6H), 1.76-1.83 (m, 2H), 1.96-2.05 (m, 4H), 2.28 (s, 3H), 2.29-2.58 (m, 10H), 3.64-3.76 (m, 6H), 4.12 (t, 4H, J=6.5 Hz).

### Example S6: Synthesis of Compound TU-005 (a Compound of Formula (11))

Compound TU-005 was prepared as shown in Scheme 6 below.

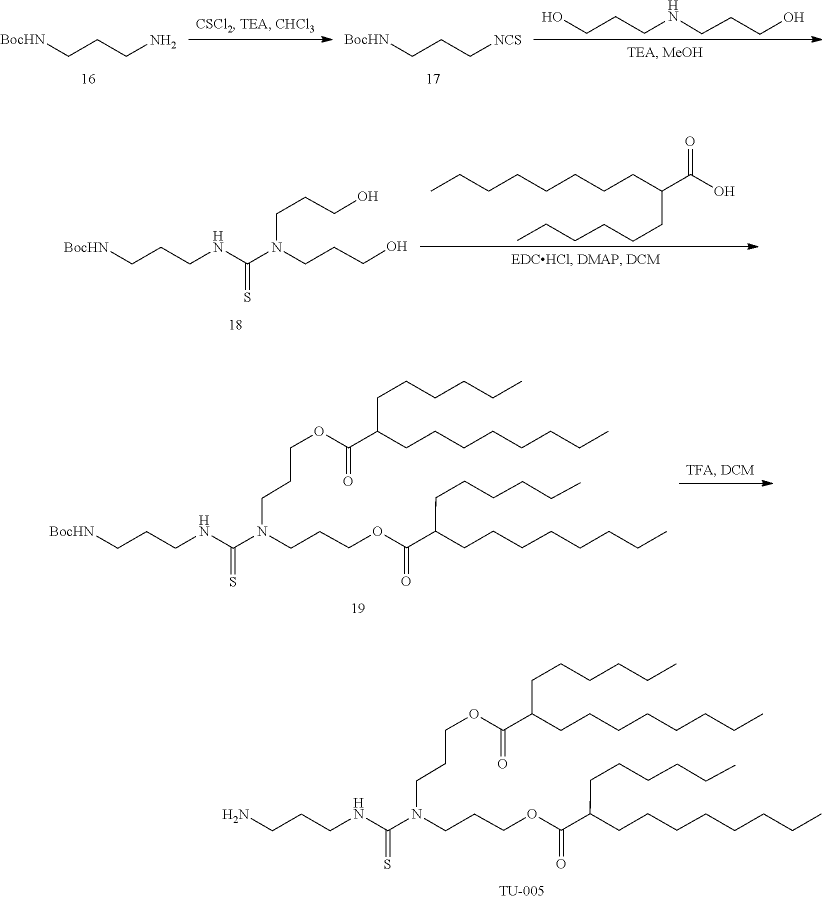

A solution of compound 16 (870 mg, 5.0 mmol) in DCM (10 mL) was cooled to 0° C. Thiophosgene (CSCl₂, 862 mg, 575 μL, 7.5 mmol) and TEA (1.4 mL, 10.0 mmol) were added to the solution. The resulting mixture was stirred at r.t. for 16 hr and then washed with sat. NaHCO₃. Compound 17 (1.08 g) was used in the next step as a crude product.

TEA (1 mL) was added to the mixture of compound 17 (1.08 g, 5.0 mmol) and bis(2-hydroxypropyl)amine (400 mg, 3.0 mmol) in MeOH (10 mL). The resulting mixture was stirred at r.t. for 16 hr until TLC indicated completion of reaction. Compound 18 (895 mg) was obtained by silica gel chromatography.

EDC·HCl (1.54 g, 8.00 mmol) and DMAP (978 mg, 8.00 mmol) were added to the mixture of compound 18 (700 mg, 2.00 mmol) and 2-hexyldecanoic acid (2.05 g, 2.34 mL, 8.00 mmol) in DCM (35 mL). The resulting mixture was stirred at r.t. for 16 hr until TLC indicated completion of reaction. Compound 19 (1.26 g) was obtained by silica gel chromatography.

A solution of compound 19 (1.20 g, 1.45 mmol) in DCM (20 mL) was cooled to 0° C. TFA (2 mL) was added and the mixture was stirred at r.t. for 16 hr. The reaction mixture was then concentrated and purified through silica gel chromatography to yield compound TU-005 (970 mg).

¹HNMR (500 MHz, CD₃OD): δ=0.92 (t, 12H, J=7.0 Hz), 1.24-1.40 (m, 40H), 1.44-1.69 (m, 8H), 1.92-2.06 (m, 6H), 2.35-2.44 (m, 2H), 3.00 (t, 2H, J=7.0 Hz), 3.77 (t, 4H, J=7.5 Hz), 3.82 (t, 2H, J=6.5 Hz), 4.15 (t, 4H, J=6.5 Hz).

### Example S7: Synthesis of Compound TU-006 (a Compound of Formula (T1))

Compound TU-006 was prepared as shown in Scheme 7 below.

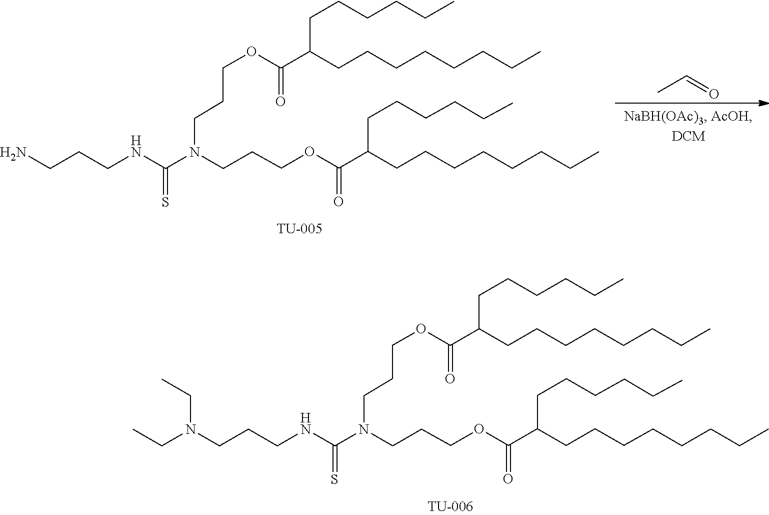

AcOH (3 μL, 0.04 mmol) was added to the mixture of compound TU-005 (120 mg, 0.16 mmol) and acetaldehyde (70 mg, 1.6 mmol, 89 L) in DCM (10 mL) and the resulting solution was stirred at r.t. for 15 min. After addition of NaBH(OAc)₃ (260 mg, 1.23 mmol) the stirring was continued for another 4 hr until TLC indicated completion of reaction. Compound TU-006 (98 mg) was obtained by silica gel chromatography.

¹HNMR (500 MHz, CD₃OD): δ=0.92 (t, 12H, J=7.0 Hz), 1.24-1.40 (m, 46H), 1.45-1.55 (m, 4H), 1.58-1.69 (m, 4H), 1.96-2.13 (m, 6H), 2.35-2.44 (m, 2H), 3.19 (t, 2H, J=7.5 Hz), 3.28 (q, 4H, J=7.0 Hz), 3.73-3.83 (m, 6H), 4.15 (t, 4H, J=6.5 Hz).

### Example S8: Synthesis of Compound TU-007 (a Compound of Formula (11))

Compound TU-007 was prepared as shown in Scheme 8 below.

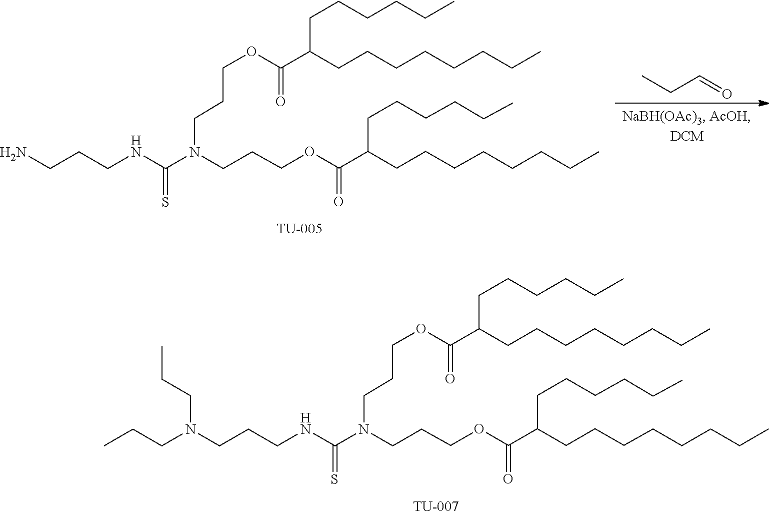

AcOH (3 μL, 0.04 mmol) was added to the mixture of compound TU-005 (120 mg, 0.16 mmol) and propionaldehyde (93 mg, 1.6 mmol, 115 μL) in DCM (10 mL) and the resulting solution was stirred at r.t. for 15 min. After addition of NaBH(OAc)₃ (260 mg, 1.23 mmol) the stirring was continued for another 4 hr until TLC indicated completion of reaction. Compound TU-007 (76 mg) was obtained by silica gel chromatography.

¹HNMR (500 MHz, CD₃OD): δ=0.92 (t, 12H, J=7.0 Hz), 1.06 (t, 6H, J=7.0 Hz), 1.23-1.38 (m, 40H), 1.46-1.67 (m, 8H), 1.72-1.84 (m, 4H), 2.00-2.19 (m, 6H), 2.35-2.45 (m, 2H), 3.09-3.19 (m, 4H), 3.22-3.29 (m, 2H), 3.52-3.67 (m, 6H), 4.11-4.24 (m, 4H).

### Example S9: Synthesis of Compound TU-008 (a Compound of Formula (11))

Compound TU-008 was prepared as shown in Scheme 9 below.

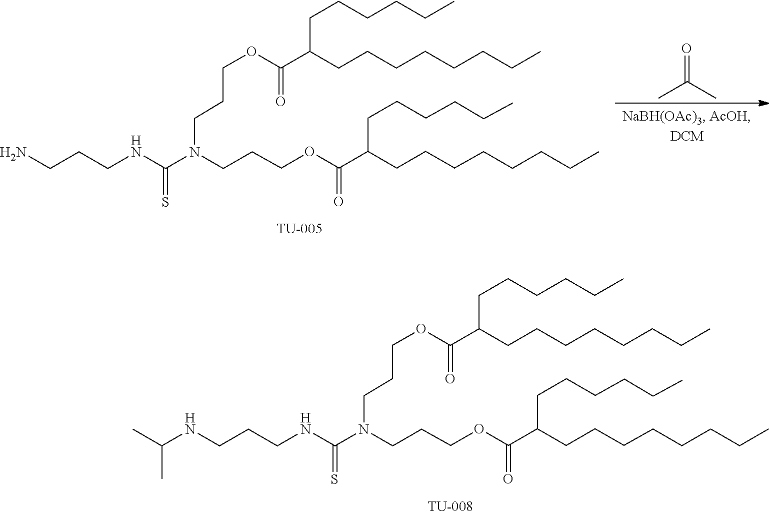

AcOH (5 μL, 0.08 mmol) was added to the mixture of compound TU-005 (250 mg, 0.34 mmol) and acetone (400 μL) in DCM (10 mL) and the resulting solution was stirred at r.t. for 15 min. After addition of NaBH(OAc)₃ (260 mg, 1.23 mmol) the stirring was continued for another 4 hr until TLC indicated completion of reaction. Compound TU-008 (206 mg) was obtained by silica gel chromatography.

¹HNMR (500 MHz, CDCl₃): δ=0.92 (t, 12H, J=7.0 Hz), 1.23-1.40 (m, 46H), 1.45-1.55 (m, 4H), 1.57-1.68 (m, 4H), 1.93-2.06 (m, 6H), 2.35-2.44 (m, 2H), 3.06 (t, 2H, J=7.0 Hz), 3.36-3.44 (m, 1H), 3.78 (t, 4H, J=7.5 Hz), 3.84 (t, 2H, J=6.5 Hz), 4.15 (t, 4H, J=6.5 Hz).

### Example S10: Synthesis of Compound TU-009 (a Compound of Formula (II))

Compound TU-009 was prepared as shown in Scheme 10 below.

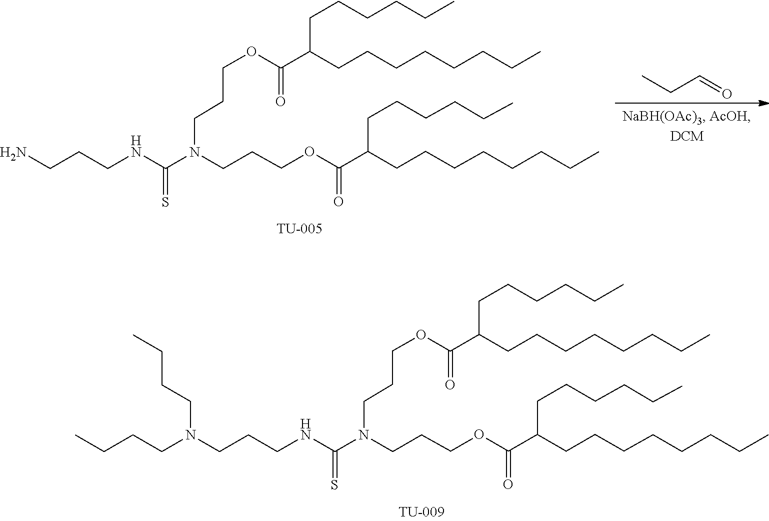

AcOH (3 μL, 0.04 mmol) was added to the mixture of compound TU-005 (120 mg, 0.16 mmol) and butyraldehyde (115 mg, 1.6 mmol, 144 μL) in DCM (10 mL) and the resulting solution was stirred at r.t. for 15 min. After addition of NaBH(OAc)₃ (260 mg, 1.23 mmol) the stirring was continued for another 4 hr until TLC indicated completion of reaction. Compound TU-009 (94 mg) was obtained by silica gel chromatography.

¹HNMR (500 MHz, CD₃OD): δ=0.92 (t, 12H, J=7.0 Hz), 1.04 (t, 6H, J=7.5 Hz), 1.24-1.38 (m, 40H), 1.41-1.55 (m, 8H), 1.58-1.77 (m, 8H), 1.97-2.11 (m, 6H), 2.36-2.43 (m, 2H), 3.15-3.24 (m, 6H), 3.73-3.81 (m, 6H), 4.15 (t, 4H, J=6.0 Hz).

### Example S11: Synthesis of Compound BAE-001 (a Compound of Formula (III))

Compound BAE-001 was prepared as shown in Scheme 11 below.

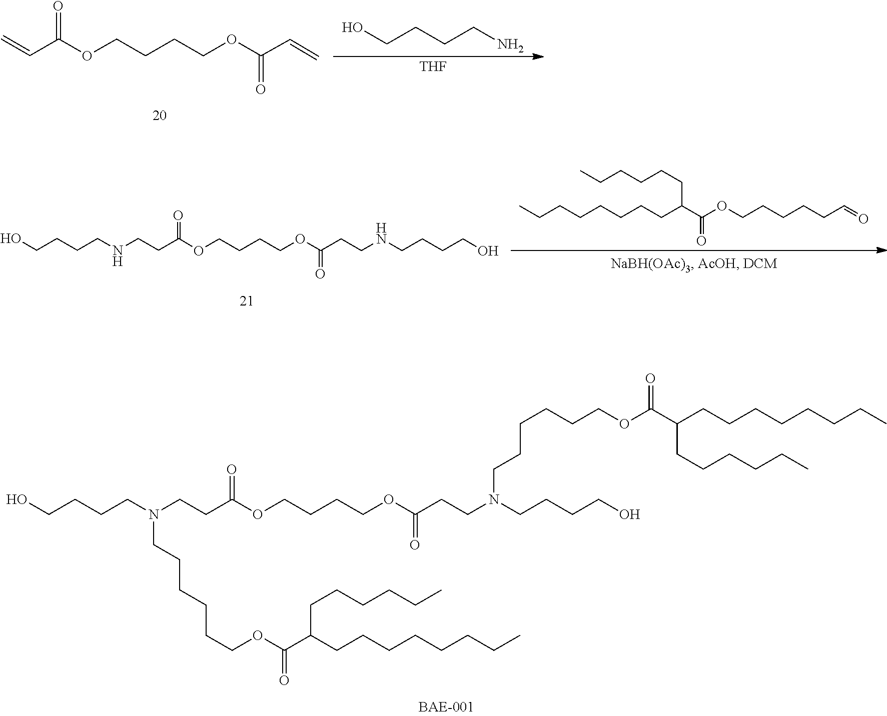

4-amino-1-butanol (450 mg, 5.04 mmol) was added to the solution of compound 20 (500 mg, 2.52 mmol) in anhydrous THF (10 mL) and the resulting mixture was stirred at r.t. for 16 hr. Compound 21 (658 mg) was obtained by silica gel chromatography.

AcOH (5 μL, 0.08 mmol) was added to the mixture of compound 21 (154 mg, 0.41 mmol) and 6-(2′-hexyldecanoyloxy)hexanal (362 mg, 1.02 mmol) in DCM (10 mL) and the resulting solution was stirred at r.t. for 15 min. After addition of NaBH(OAc)₃ (260 mg, 1.23 mmol) the stirring was continued for another 4 hr until TLC indicated completion of reaction. Compound BAE-001 (98 mg) was obtained by silica gel chromatography.

¹HNMR (500 MHz, CDCl₃): δ=0.87 (t, 12H, J=7.0 Hz), 1.17-1.80 (m, 76H), 2.27-2.34 (m, 2H), 2.36-2.62 (br, 12H), 2.75-2.97 (m, 4H), 3.52-3.61 (m, 4H), 4.05 (t, 4H, J=6.5 Hz), 4.08-4.15 (m, 4H).

### Example S12: Synthesis of Compound BAA-001 (a Compound of Formula (III))

Compound BAA-001 was prepared as shown in Scheme 12 below.

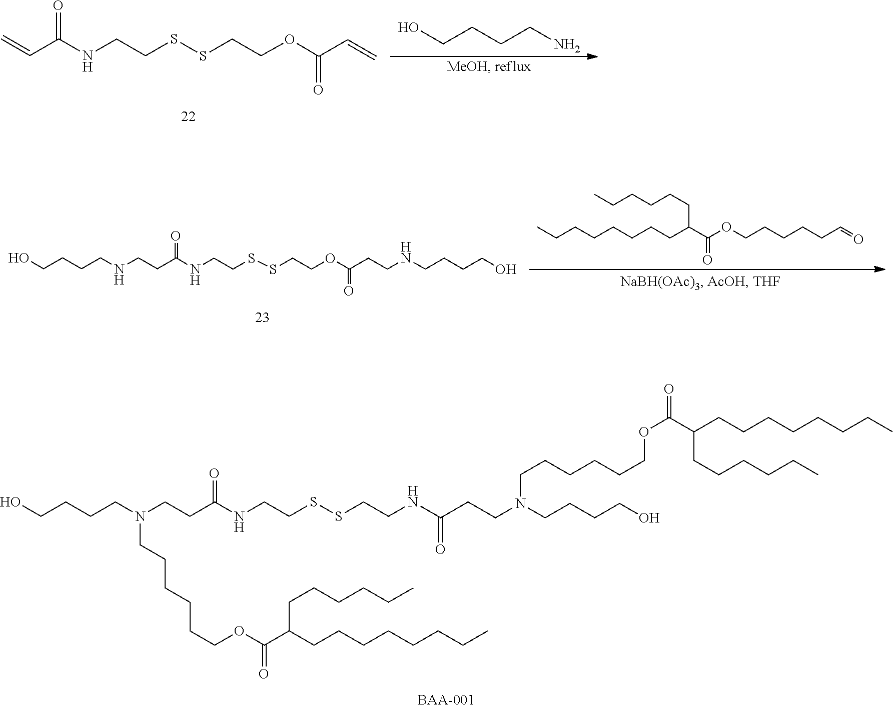

4-amino-1-butanol (450 mg, 5.04 mmol) was added to the solution of compound 22 (522 mg, 2.00 mmol) in anhydrous methanol (20 mL) and the resulting mixture was refluxed for 16 hr. Compound 23 (705 mg) was obtained by C18 gel chromatography.

AcOH (5 μL, 0.08 mmol) was added to the mixture of compound 23 (180 mg, 0.41 mmol) and 6-(2′-hexyldecanoyloxy)hexanal (362 mg, 1.02 mmol) in THE (10 mL) and the resulting solution was stirred at r.t. for 15 min. After addition of NaBH(OAc)₃ (260 mg, 1.23 mmol) the stirring was continued for another 4 hr until TLC indicated completion of reaction. Compound BAA-001 (213 mg) was obtained by silica gel chromatography.

¹HNMR (500 MHz, CD₃OD): δ=0.92 (t, 12H, J=7.0 Hz), 1.23-1.72 (m, 74H), 2.32-2.41 (m, 6H), 2.47-2.54 (m, 6H), 2.81 (t, 4H, J=7.0 Hz), 2.85 (t, 4H, J=7.0 Hz), 3.52 (t, 4H, J=6.5 Hz), 3.58 (t, 4H, J=6.0 Hz), 4.11 (t, 4H, J=6.5 Hz).

### Example S13: Synthesis of Compound DS-001 (a Compound of Formula (IV))

Compound DS-001 was prepared as shown in Scheme 13 below.

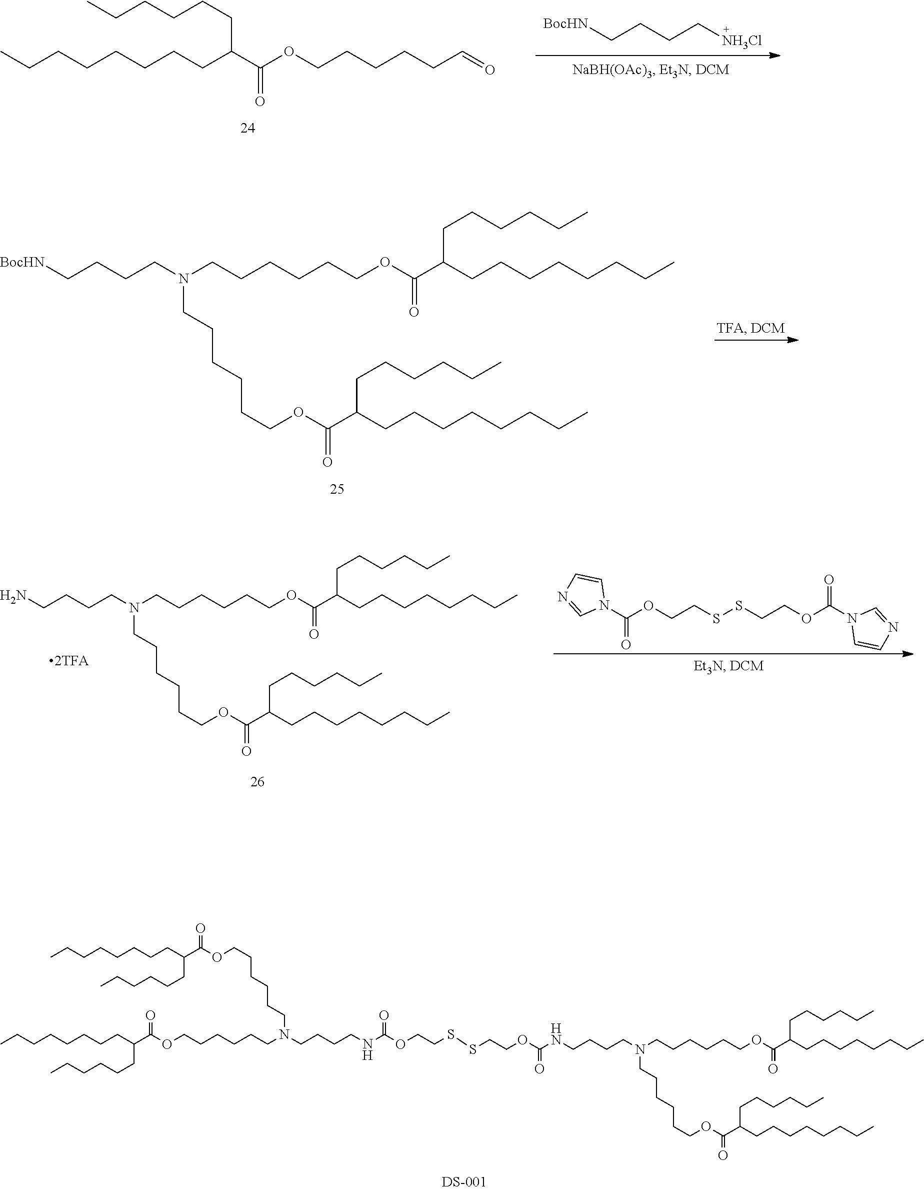

TEA (90 mg, 124 μL, 0.89 mmol) was added to a mixture of compound 24 (1.58 g, 4.45 mmol) and N-Boc-1,4-butanediammonium hydrochloride (400 mg, 1.78 mmol) in DCM (30 mL). The resulting solution was stirred at r.t. for 15 min followed by addition of NaBH(OAc)₃ (1.13 g, 5.34 mmol) and stirred for another 4 hr until TLC indicated completion of reaction. Compound 25 (1.3 g) was obtained by silica gel chromatography.

TFA (3 mL) was added to the solution of compound 25 (400 mg, 0.46 mmol) in DCM (6 mL). The resulting mixture was stirred at r.t. for 16 hr until TLC indicated completion of reaction. Compound 26 (429 mg) was obtained by elution through a C-18 gel cartridge.

TEA (0.5 mL) was added to a mixture of compound 26 (200 mg, 0.21 mmol) and 2-imidazolylcarbonyloxyethyl disulfide (32.8 mg, 0.096 mmol) in DCM (6 mL). The resulting mixture was stirred at r.t. for 16 hr until TLC indicated completion of reaction. Compound DS-001 was obtained by elution through a C-18 gel cartridge.

¹HNMR (500 MHz, CDCl₃): δ=0.84-0.90 (m, 24H), 1.19-1.66 (m, 136H), 2.26-2.34 (m, 4H), 2.34-2.42 (m, 12H), 2.88-2.96 (m, 4H), 3.12-3.21 (m, 4H), 4.06 (t, 8H, J=7.0 Hz), 4.30 (t, 4H, J=7.0 Hz).

### Example 14: Characterization of KT-001, TU-001, TU-002, TU-003, TU-004, TU-005, TU-006, TU-007, TU-008, TU-009, BAE-001, BAA-001, and DS-001

Lipid nanoparticles were prepared with the following compositions:
ALC-0315:DSPC:Cholesterol:GM020=50:10:38.5:1.5 (and buffer) (positive control)
TU-001:DSPC:Cholesterol:GM020=50:10:38.5:1.5 (and buffer)
TU-002:DSPC:Cholesterol:GM020=50:10:38.5:1.5 (and buffer)
TU-003:DSPC:Cholesterol:GM020=50:10:38.5:1.5 (and buffer)
TU-004:DSPC:Cholesterol:GM020=50:10:38.5:1.5 (and buffer)
TU-005:DSPC:Cholesterol:GM020=50:10:38.5:1.5 (and buffer)
TU-006:DSPC:Cholesterol:GM020=50:10:38.5:1.5 (and buffer)
TU-007:DSPC:Cholesterol:GM020=50:10:38.5:1.5 (and buffer)
TU-008:DSPC:Cholesterol:GM020=50:10:38.5:1.5 (and buffer)
TU-009:DSPC:Cholesterol:GM020=50:10:38.5:1.5 (and buffer)
BAE-001:DSPC:Cholesterol:GM020=50:10:38.5:1.5 (and buffer)
BAA-001:DSPC:Cholesterol:GM020=50:10:38.5:1.5 (and buffer)
DS-001:DSPC:Cholesterol:GM020=25:10:38.5:1.5 (and buffer)
KT-001:DSPC:Cholesterol:GM020=50:10:38.5:1.5 (and buffer)
Where buffer is water, Trehalose, Tris Base, sodium chloride. DSPC is 1,2-distearoyl-sn-glycero-3-phosphocholine. GM020 is DMG-PEG (1,2-Dimyristoyl-rac-glycero-3-methylpolyoxyethylene).

Table 1 summarizes the lipid nanoparticle characterization with respect to various parameters including nanoparticle size (Z avg), polydispersity index (PDI), charge (Zeta potential), encapsulation efficiency and yield.

TABLE 1

| | | Fresh Samples | | | | | | |
|---|---|---|---|---|---|---|---|---|
| | Amount of | | | Zeta | Encapsulation | Conc. of | Total | |
| | mRNA | Z Avg. | | Potential | Efficiency | mRNA | volume | Yield |
| Composition | (μg) | (nm) | PDI | (mV) | (%)* | (μg/mL) | (mL) | (%) |
| ALC-0315 (rsF352) | 250 | 78.67 | 0.223 | −4.74 | 83.16 | 116.16 | 1.82 | 84.6 |
| TU-001 (rsF348) | 250 | 73.81 | 0.208 | 0.0305 | 66.04 | 86.16 | 1.87 | 64.4 |
| TU-002 (rsF350) | 250 | 76.99 | 0.133 | −2.55 | 80.64 | 118.38 | 1.26 | 59.7 |
| TU-003 (rsF384) | 250 | 73.29 | 0.23 | −0.56 | 90.47 | 117.76 | 1.75 | 82.43 |
| TU-004 (rsF386) | 250 | 79.77 | 0.24 | −1.27 | 72.53 | 110.22 | 1.75 | 77.16 |
| TU-005 | 250 | 176.9 | 0.282 | −0.842 | 97.66 | 56.46 | 2.90 | 65.49 |
| TU-006 | 250 | 68.84 | 0.278 | −0.642 | 81.48 | 56.70 | 3.13 | 70.99 |
| TU-007 | 250 | 63.30 | 0.156 | −2.68 | 78.96 | 49.35 | 3.56 | 70.27 |
| TU-008 | 250 | 64.64 | 0.253 | −1.07 | 97.75 | 51.63 | 3.18 | 65.67 |
| TU-009 | 250 | 64.82 | 0.188 | −4.40 | 80.76 | 56.78 | 3.02 | 68.59 |
| BAE-001 (rsF359) | 250 | 111.7 | 0.141 | −0.28 | 83.32 | 107.06 | 2.04 | 87.4 |
| BAA-001 | 250 | 85.77 | 0.015 | −2.02 | 72.79 | 78.70 | 2.22 | 69.89 |
| DS-001 (rsF373) | 250 | 63.0 | 0.12 | −3.74 | 84.50 | 91.42 | 1.85 | 67.6 |
| KT-001 (rsF377) | 250 | 84.7 | 0.08 | −3.30 | 80.60 | 98.20 | 1.90 | 74.6 |

### BIOLOGICAL EXAMPLES

### Example B¹: In Vivo Efficacy of SARS-CoV-2 Spike mRNA in Lipid Nanoparticle Formulation

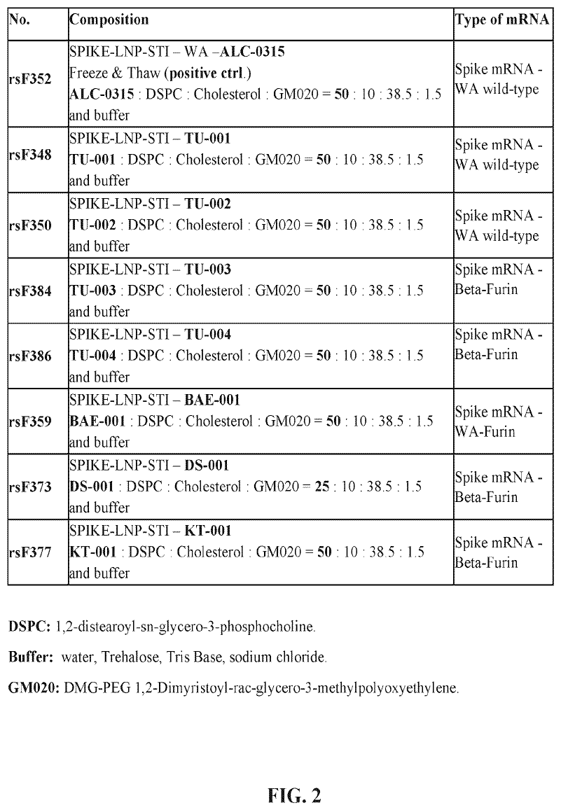

Eight formulations containing SARS-CoV-2 spike mRNA in lipid nanoparticles were prepared. All formulations had the same mRNA concentration of 0.02 μg/pL, and the same volume of (50 μL) was injected into each mouse. Specific formulation of each lipid nanoparticle is shown in FIG. 2. The rsF352 formulation comprising ALC-0315 was a positive control or reference formulation.

All the Spike mRNA LNP (lipid nanoparticle) formulations were administered via intramuscular injections to female Balb/c mice. The female Balb/c mice (6-8 weeks old, 16-20 g) were purchased from The Jackson Laboratory (USA). Each of the six formulations was administered to 5 mice (i.e., a total of 30 mice). On day 7, day 14, and day 28 post injection, blood was collected from each mouse.

Mouse serum samples were isolated using refrigerated tabletop centrifuge (10 k RCF, 4° C., 20 min). Following the order of mouse ear tag number, samples were transferred to serum storage plate, and stored at −70° C. before ELISA.

ELISA was performed following Spike S1 mIgG ELISA Protocol, according to standard techniques (e.g., https://www.sinobiological.com/antibodies/cov-spike-40592-mm117).

GraphPad Prism 9 was used to do 4PL curve fitting and interpolation to calculate the IgG concentrations in mouse serum samples. Values were presented as mean±SEM.

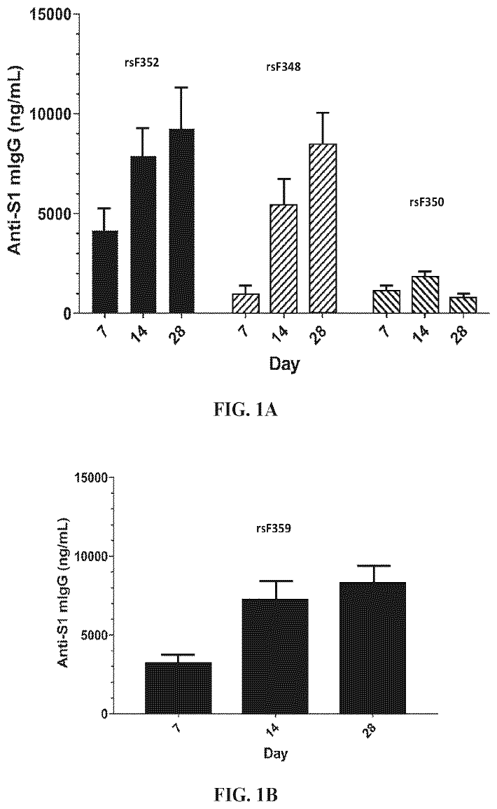

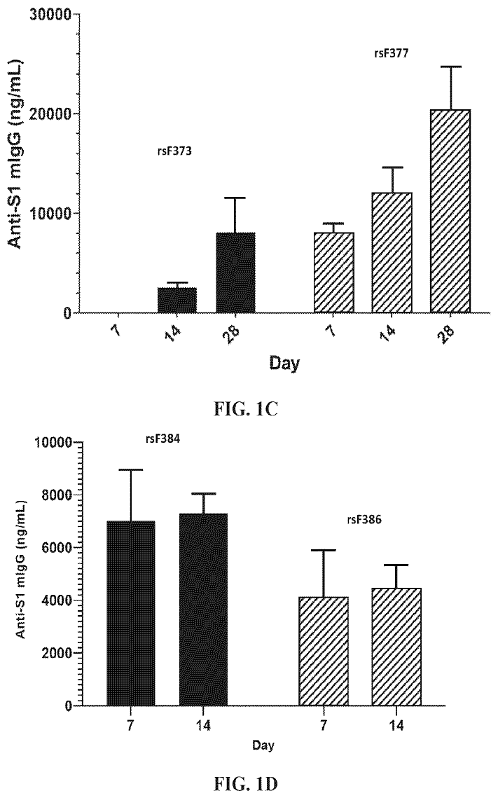

FIGS. 1A-D show the amount of anti-Spike antibody (of SARS-CoV-2) produced in mice, in response to injection of lipid nanoparticles comprising Spike mRNA. FIG. 1A demonstrates that comparable amounts of anti-Spike antibody (of SARS-CoV-2) were produced in mice 28 days following injection of lipid nanoparticles comprising ALC-0315 lipids (positive control), and lipid nanoparticles comprising TU-001 lipids, where the rest of the nanoparticle composition was the same (see FIG. 2) (lipid nanoparticles in FIG. 1A comprise Washington (WA) wild-type Spike mRNA).

FIGS. 1C-D demonstrate that the largest amounts of anti-Spike antibody (of SARS-CoV-2) were produced in mice injected with lipid nanoparticles comprising KT-001 lipids (28 days following injection; lipid nanoparticles in FIGS. 1C-D comprise Beta-Furin Spike mRNA).

The complete disclosures of all publications cited herein are incorporated herein by reference in their entireties as if each were individually set forth in full herein and incorporated.

Various modifications and alterations to the embodiments disclosed herein will become apparent to those skilled in the art without departing from the scope and spirit of this disclosure. Illustrative embodiments and examples are provided as examples only and are not intended to limit the scope of the present invention.

1. A cationic lipid of formula (I): 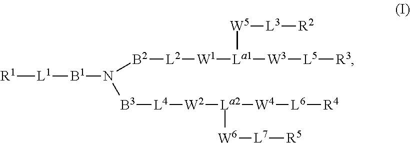
or a pharmaceutically acceptable salt, solvate, hydrate, stereoisomer, or prodrug thereof,
wherein:
R¹ is H, —OR^{1A}, —YOR^{1A}, —NR^{1A}R^{1B}, —YNR^{1A}R^{1B}, —SR^{1A}, —YSR^{1A}, —(C═O)R^{1A}, —Y(C═O)R^{1A}, —(C═O)OR^{1A}, —Y(C═O)OR^{1A}, —O(C═O)R^{1A}, —YO(C═O)R^{1A}, —O(C═O)OR^{1A}, —YO(C═O)OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Y is substituted or unsubstituted C₀-C₁₂ alkylene or substituted or unsubstituted 0- to 12-membered heteroalkylene;
R² is H, —OR^{2A}, —SR^{2A}, —(C═O)R^{2A}, —(C═O)OR^{2A}, —O(C═O)R^{2A}, —O(C═O)OR^{2A}, —(C═O)NHR^{2A}, —NH(C═O)R^{2A}, substituted or unsubstituted alkyl, or substituted or unsubstituted heteroalkyl;
R³ is H, —OR^{3A}, —SR^{3A}, —(C═O)R^{3A}, —(C═O)OR^{3A}, —O(C═O)R^{3A}, —O(C═O)OR^{3A}, —(C═O)NHR^{3A}, —NH(C═O)R^{3A}, substituted or unsubstituted alkyl, or substituted or unsubstituted heteroalkyl;
R⁴ is H, —OR^{4A}, —SR^{4A}, —(C═O)R^{4A}, —(C═O)OR^{4A}, —O(C═O)R^{4A}, —O(C═O)OR^{4A}, —(C═O)NHR^{4A}, —NH(C═O)R^{4A}, substituted or unsubstituted alkyl, or substituted or unsubstituted heteroalkyl;
R⁵ is H, —OR^{5A}, —SR^{5A}, —(C═O)R^{5A}, —(C═O)OR^{5A}, —O(C═O)R^{5A}, —O(C═O)OR^{5A}, —(C═O)NHR^{5A}, —NH(C═O)R^{5A}, substituted or unsubstituted alkyl, or substituted or unsubstituted heteroalkyl;
B¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
B² and B³ are each independently a bond, substituted or unsubstituted alkylene, or substituted or unsubstituted heteroalkylene;
L¹ is a bond, —O(C═O)—, —(C═O)O—, —O(C═O)O—, —C(═O)—, —O—, —O(CR¹⁰¹R¹⁰²)SO—, —S—, —C(═O)S—, —SC(═O)—, —NR¹⁰¹C(═O)—, —C(═O)NR¹⁰¹—, —NR¹⁰¹C(═S)—, —C(═S)NR¹⁰¹—, —NR¹⁰¹C(═O)NR¹⁰²—, —NR¹⁰¹C(═S)NR¹⁰²—, —OC(═O)NR¹⁰¹—, —NR¹⁰¹C(═O)O—, —SC(═O)NR¹⁰¹— or —NR¹⁰¹C(═O)S—;
L² is a bond, —O(C═O)—, —(C═O)O—, —O(C═O)O—, —C(═O)—, —O—, —O(CR²⁰¹R²⁰²)ₛO—, —S—, —C(═O)S—, —SC(═O)—, —NR²⁰¹C(═O)—, —C(═O)NR²⁰¹—, —NR²⁰¹C(═O)NR²⁰²—, —NR²⁰¹C(═S)—, —C(═S)NR²⁰¹—, —NR²⁰¹C(═S)NR²⁰²—, —OC(═O)NR²⁰¹—, —NR²⁰¹C(═O)O—, —SC(═O)NR²⁰¹— or —NR²⁰¹C(═O)S—;
L³ is a bond, —O(C═O)—, —(C═O)O—, —O(C═O)O—, —C(═O)—, —O—, —O(CR³⁰¹R³⁰²)ₛO—, —S—, —C(═O)S—, —SC(═O)—, —NR³⁰¹C(═O)—, —C(═O)NR³⁰¹—, —NR³⁰¹C(═O)NR³⁰²—, —NR³⁰¹C(═S)—, —C(═S)NR³⁰¹—, —NR³⁰¹C(═S)NR³⁰²—, —OC(═O)NR³⁰¹—, —NR³⁰¹C(═O)O—, —SC(═O)NR³⁰¹— or —NR³⁰¹C(═O)S—;
L⁴ is a bond, —O(C═O)—, —(C═O)O—, —O(C═O)O—, —C(═O)—, —O—, —O(CR⁴⁰¹R⁴⁰²)ₛO—, —S—, —C(═O)S—, —SC(═O)—, —NR⁴⁰¹C(═O)—, —C(═O)NR⁴⁰¹—, —NR⁴⁰¹C(═O)NR⁴⁰²—, —NR⁴⁰¹C(═S)—, —C(═S)NR⁴⁰¹—, —NR⁴⁰¹C(═S)NR⁴⁰²—, —OC(═O)NR⁴⁰¹—, —NR⁴⁰¹C(═O)O—, —SC(═O)NR⁴⁰¹— or —NR⁴⁰¹C(═O)S—;
L⁵ is a bond, —O(C═O)—, —(C═O)O—, —O(C═O)O—, —C(═O)—, —O—, —O(CR⁵⁰¹R⁵⁰²)ₛO—, —S—, —C(═O)S—, —SC(═O)—, —NR⁵⁰¹C(═O)—, —C(═O)NR⁵⁰¹—, —NR⁵⁰¹C(═O)NR⁵⁰²—, —NR⁵⁰¹C(═S)—, —C(═S)NR⁵⁰¹—, —NR⁵⁰¹C(═S)NR⁵⁰²—, —OC(═O)NR⁵⁰¹—, —NR⁵⁰¹C(═O)O—, —SC(═O)NR⁵⁰¹— or —NR⁵⁰¹C(═O)S—;
L⁶ is a bond, —O(C═O)—, —(C═O)O—, —O(C═O)O—, —C(═O)—, —O—, —O(CR⁶⁰¹R⁶⁰²)ₛO—, —S—, —C(═O)S—, —SC(═O)—, —NR⁶⁰¹C(═O)—, —C(═O)NR⁶⁰¹—, —NR⁶⁰¹C(═O)NR⁶⁰²—, —NR⁶⁰¹C(═S)—, —C(═S)NR⁶⁰¹—, —NR⁶⁰¹C(═S)NR⁶⁰²—, —OC(═O)NR⁶⁰¹—, —NR⁶⁰¹C(═O)O—, —SC(═O)NR⁶⁰¹— or —NR⁶⁰¹C(═O)S—;
L⁷ is a bond, —O(C═O)—, —(C═O)O—, —O(C═O)O—, —C(═O)—, —O—, —O(CR⁷⁰¹R⁷⁰²)ₛO—, —S—, —C(═O)S—, —SC(═O)—, —NR⁷⁰¹C(═O)—, —C(═O)NR⁷⁰¹—, —NR⁷⁰¹C(═O)NR⁷⁰²—, —NR⁷⁰¹C(═S)—, —C(═S)NR⁷⁰¹—, —NR⁷⁰¹C(═S)NR⁷⁰²—, —OC(═O)NR⁷⁰¹—, —NR⁷⁰¹C(═O)O—, —SC(═O)NR⁷⁰¹— or —NR⁷⁰¹C(═O)S—;
L^{a1} and L^{a2} are each independently 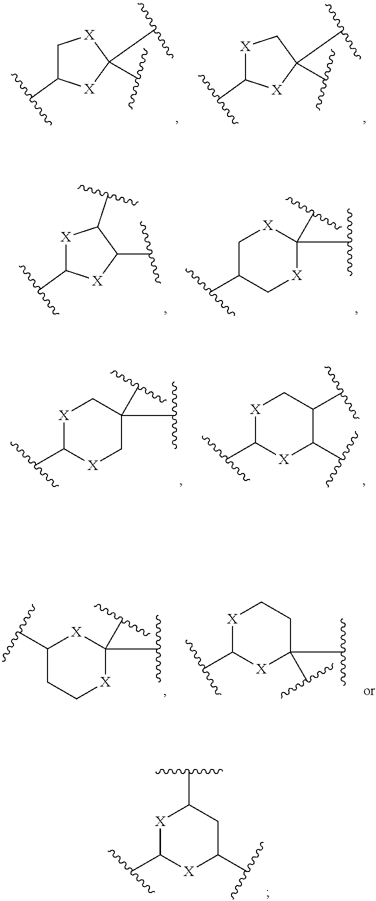
each X is independently O, S, or CH₂;
W¹, W², W³, W⁴, W⁵, and W⁶ are each independently a bond, substituted or unsubstituted C₁-C₁₂ alkylene, or substituted or unsubstituted 2- to 12-membered heteroalkylene;
each R^{1A} and R^{1B} is independently H, substituted or unsubstituted C₁-C₁₂ alkyl, or substituted or unsubstituted 2- to 12-membered heteroalkyl;
each R^{2A}, R^{3A}, R^{4A}, and R^{5A} is independently H, substituted or unsubstituted C₁-C₃₀ alkyl, or substituted or unsubstituted 2- to 30-membered heteroalkyl;
each R¹⁰¹, R¹⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently H, substituted or unsubstituted C₁-C₁₂ alkyl, or substituted or unsubstituted 2- to 12-membered heteroalkyl; and
each s is independently an integer from 1 to 4.

2. The cationic lipid or pharmaceutically acceptable salt, solvate, hydrate, stereoisomer, or prodrug thereof of claim 1, wherein:
R¹ is H, —OR^{1A}, or substituted or unsubstituted heteroalkyl;
L¹ is a bond, —NR¹⁰¹C(═S)—, —C(═S)NR¹⁰¹—, —O(C═O)—, —(C═O)O—, or —O—;
B¹ is a bond or a substituted or unsubstituted alkylene;
B² and B³ are each independently a bond or substituted or unsubstituted alkylene;
L² is a bond, —O(C═O)—, —(C═O)O—, —O(C═O)O—, —C(═O)—, —O—, or —S—;
L⁴ is a bond, —O(C═O)—, —(C═O)O—, —O(C═O)O—, —C(═O)—, —O—, or —S—;
W¹, W², W³, W⁴, W⁵, and W⁶ are each independently a bond or substituted or unsubstituted C₁-C₁₂ alkylene;
L^{a1} and L^{a2} are each independently 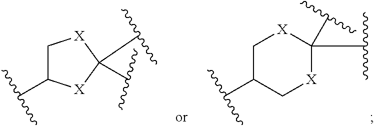
each X is independently O or S;
L³ is a bond, —O(C═O)—, —(C═O)O—, —O(C═O)O—, —C(═O)—, —O—, or —S—;
L⁵ is a bond, —O(C═O)—, —(C═O)O—, —O(C═O)O—, —C(═O)—, —O—, or —S—;
L⁶ is a bond, —O(C═O)—, —(C═O)O—, —O(C═O)O—, —C(═O)—, —O—, or —S—;
L⁷ is a bond, —O(C═O)—, —(C═O)O—, —O(C═O)O—, —C(═O)—, —O—, or —S—;
R² is H or substituted or unsubstituted alkyl;
R³ is H or substituted or unsubstituted alkyl;
R⁴ is H or substituted or unsubstituted alkyl;
R⁵ is H or substituted or unsubstituted alkyl;
each R^{1A} is independently H or substituted or unsubstituted C₁-C₁₂ alkyl; and
each R¹⁰¹ is independently H or substituted or unsubstituted 2- to 12-membered heteroalkyl.

3. The cationic lipid or pharmaceutically acceptable salt, solvate, hydrate, stereoisomer, or prodrug thereof of claim 1, wherein:
R¹ is H, —OH, methoxy, ethoxy, or substituted or unsubstituted heteroalkyl;
L¹ is a bond, —NR¹⁰¹C(═S)—, or —C(═S)NR¹⁰¹—;
B¹ is a bond or an unsubstituted C₁-C₅ alkylene;
B² and B³ are each independently a bond or substituted or unsubstituted C₁-C₈ alkylene;
L² is a bond, —O(C═O)—, or —(C═O)O—;
L⁴ is a bond, —O(C═O)—, or —(C═O)O—;
W¹, W², W³, W⁴, W⁵, and W⁶ are each independently a bond or substituted or unsubstituted C₁-C₁₂ alkylene;
L^{a1} and L^{a2} are each independently 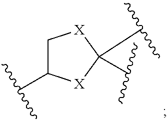
each X is independently O or S;
L³ is a bond, —O(C═O)—, or —(C═O)O—;
L⁵ is a bond, —O(C═O)—, or —(C═O)O—;
L⁶ is a bond, —O(C═O)—, or —(C═O)O—;
L⁷ is a bond, —O(C═O)—, or —(C═O)O—;
R² is H or substituted or unsubstituted C₁-C₁₂ alkyl;
R³ is H or substituted or unsubstituted C₁-C₁₂ alkyl;
R⁴ is H or substituted or unsubstituted C₁-C₁₂ alkyl;
R⁵ is H or substituted or unsubstituted C₁-C₁₂ alkyl; and
each R¹⁰¹ is independently substituted or unsubstituted 2- to 12-membered heteroalkyl.

4. The cationic lipid or pharmaceutically acceptable salt, solvate, hydrate, stereoisomer, or prodrug thereof of claim 1, wherein:
R¹ is —OH or methoxy;
L¹ is a bond;
B¹ is an unsubstituted C₁-C₈ alkylene;
B² and B³ are each independently a bond or substituted or unsubstituted C₁-C₈ alkylene;
L² is a bond;
L⁴ is a bond;
W¹, W², W³, W⁴, W⁵, and W⁶ are each independently a bond or substituted or unsubstituted C₁-C₁₂ alkylene;
L^{a1} and L^{a2} are each independently 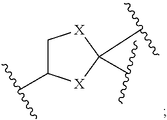
each X is independently O;
L³ is a bond;
L⁵ is a bond;
L⁶ is a bond;
L⁷ is a bond;
R² is H or substituted or unsubstituted C₁-C₁₂ alkyl;
R³ is H or substituted or unsubstituted C₁-C₁₂ alkyl;
R⁴ is H or substituted or unsubstituted C₁-C₁₂ alkyl; and
R⁵ is H or substituted or unsubstituted C₁-C₁₂ alkyl.

5. The cationic lipid or pharmaceutically acceptable salt, solvate, hydrate, stereoisomer, or prodrug thereof of claim 1, wherein:
R¹ is substituted or unsubstituted heteroalkyl;
L¹ is —C(═S)NR¹⁰¹—, where the carbon atom is connected to the nitrogen atom in formula (I);
B¹ is a bond;
B² and B³ are each independently a bond or substituted or unsubstituted C₁-C₈ alkylene;
L² is a bond, —O(C═O)—, or —(C═O)O—;
L⁴ is a bond, —O(C═O)—, or —(C═O)O—;
W¹, W², W³, W⁴, W⁵, and W⁶ are each independently a bond or substituted or unsubstituted C₁-C₁₂ alkylene;
L^{a1} and L^{a2} are each independently; 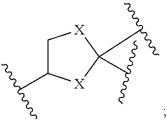
each X is independently O;
L³ is a bond;
L⁵ is a bond;
L⁶ is a bond;
L⁷ is a bond;
R² is H or substituted or unsubstituted C₁-C₁₂ alkyl;
R³ is H or substituted or unsubstituted C₁-C₁₂ alkyl;
R⁴ is H or substituted or unsubstituted C₁-C₁₂ alkyl; and
R⁵ is H or substituted or unsubstituted C₁-C₁₂ alkyl.

6. The cationic lipid or pharmaceutically acceptable salt, solvate, hydrate, stereoisomer, or prodrug thereof of claim 1, wherein the cationic lipid is: 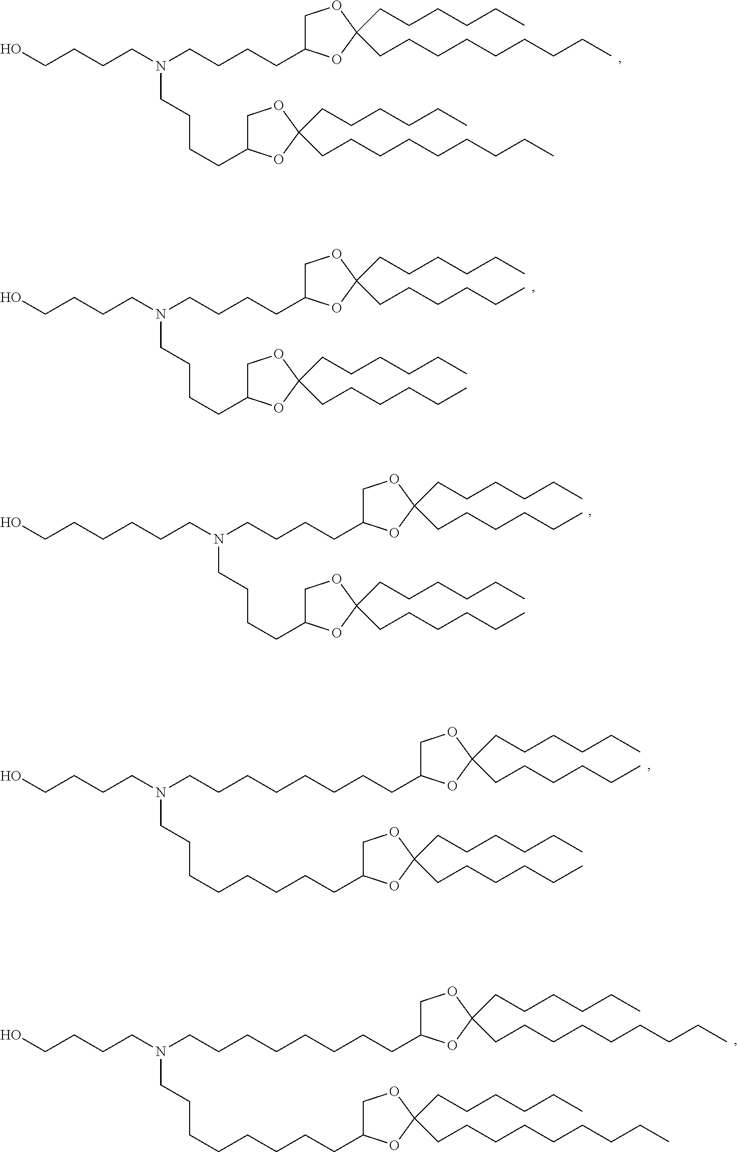 [SMILES: CCCCCCCCCC1(CCCCCC)OCC(CCCCN(CCCCO)CCCCC2COC(CCCCCC)(CCCCCCCCC)O2)O1 ; CCCCCCC1(CCCCCC)OCC(CCCCN(CCCCO)CCCCC2COC(CCCCCC)(CCCCCC)O2)O1 ; CCCCCCC1(CCCCCC)OCC(CCCCN(CCCCCCO)CCCCC2COC(CCCCCC)(CCCCCC)O2)O1 ; CCCCCCC1(CCCCCC)OCC(CCCCCCCCN(CCCCO)CCCCCCCCC2COC(CCCCCC)(CCCCCC)O2)O1 ; CCCCCCCCCC1(CCCCCC)OCC(CCCCCCCCN(CCCCO)CCCCCCCCC2COC(CCCCCC)(CCCCCCCCC)O2)O1] 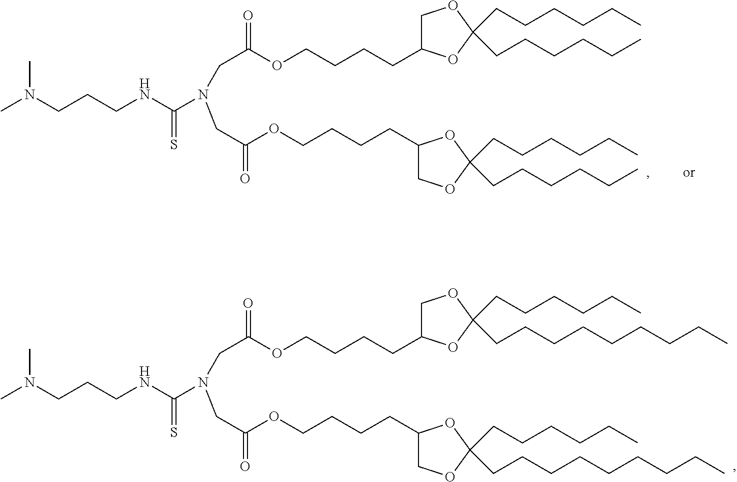
or a pharmaceutically acceptable salt thereof.

7. A cationic lipid of formula (II): 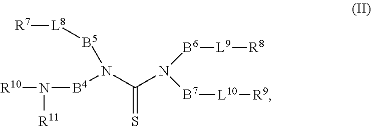
or a pharmaceutically acceptable salt, solvate, hydrate, stereoisomer, or prodrug thereof,
wherein:
B⁴ is W⁷-L^{a3}-W⁸;
W⁷ and W⁸ are each independently a bond, substituted or unsubstituted alkylene, or substituted or unsubstituted heteroalkylene;
L^{a3} is a bond, —O(C═O)—, —(C═O)O—, —O(C═O)O—, —C(═O)—, —O—, —O(CR^{a31}R^{a32})SO—, —S—, —C(═O)S—, —SC(═O)—, —NR^{a31}C(═O)—, —C(═O)NR^{a31}—, —NR^{a31}C(═O)NR^{a32}—, —NR^{a31}C(═S)—, —C(═S)NR^{a31}—, —NR^{a31}C(═S)NR^{a32}—, —OC(═O)NR^{a31}—, —NR^{a31}C(═O)O—, —SC(═O)NR^{a31}— or —NR^{a31}C(═O)S—;
R¹⁰ and R¹¹ are each independently H, substituted or unsubstituted alkyl, substituted or unsubstituted heteroalkyl, or R¹⁰ and R¹¹ together with the nitrogen atom to which they are connected form a substituted or unsubstituted heterocycloalkyl or substituted or unsubstituted heteroaryl;
B⁵, B⁶, and B⁷ are each independently a bond, substituted or unsubstituted alkylene, or substituted or unsubstituted heteroalkylene;
L⁸ is a bond, —O(C═O)—, —(C═O)O—, —O(C═O)O—, —C(═O)—, —O—, —O(CR⁸⁰¹R⁸⁰²)SO—, —S—, —C(═O)S—, —SC(═O)—, —NR⁸⁰¹C(═O)—, —C(═O)NR⁸⁰¹—, —NR⁸⁰¹C(═O)NR⁸⁰²—, —NR⁸⁰¹C(═S)—, —C(═S)NR⁸⁰¹—, —NR⁸⁰¹C(═S)NR⁸⁰²—, —OC(═O)NR⁸⁰¹—, —NR⁸⁰¹C(═O)O—, —SC(═O)NR⁸⁰¹— or —NR⁸⁰¹C(═O)S—;
L⁹ is a bond, —O(C═O)—, —(C═O)O—, —O(C═O)O—, —C(═O)—, —O—, —O(CR⁹⁰¹R⁹⁰²)ₛO—, —S—, —C(═O)S—, —SC(═O)—, —NR⁹⁰¹C(═O)—, —C(═O)NR⁹⁰¹—, —NR⁹⁰¹C(═O)NR⁹⁰²—, —NR⁹⁰¹C(═S)—, —C(═S)NR⁹⁰¹—, —NR⁹⁰¹C(═S)NR⁹⁰²—, —OC(═O)NR⁹⁰¹—, —NR⁹⁰¹C(═O)O—, —SC(═O)NR⁹⁰¹— or —NR⁹⁰¹C(═O)S—;
L¹⁰ is a bond, —O(C═O)—, —(C═O)O—, —O(C═O)O—, —C(═O)—, —O—, —O(CR¹¹⁰R¹¹¹)SO—, —S—, —C(═O)S—, —SC(═O)—, —NR¹¹⁰C(═O)—, —C(═O)NR¹¹⁰—, —NR¹¹⁰C(═O)NR¹¹¹—, —NR¹¹⁰C(═S)—, —C(═S)NR¹¹⁰—, —NR¹¹⁰C(═S)NR¹¹¹—, —OC(═O)NR¹¹⁰—, —NR¹¹⁰C(═O)O—, —SC(═O)NR¹¹⁰— or —NR¹¹⁰C(═O)S—;
R⁷, R⁸, and R⁹ are each independently H, substituted or unsubstituted C₁-C₃₀ alkyl, or substituted or unsubstituted 2- to 30-membered heteroalkyl;
each R^{a31} and R^{a32} is independently H, substituted or unsubstituted C₁-C₁₂ alkyl, or substituted or unsubstituted 2- to 12-membered heteroalkyl;
each R⁸⁰¹, R⁸⁰², R⁹⁰¹, R⁹⁰², R¹¹⁰, and R¹¹¹ is independently H, substituted or unsubstituted C₁-C₁₂ alkyl, or substituted or unsubstituted 2- to 12-membered heteroalkyl; and
each s is independently an integer from 1 to 4.

8. The cationic lipid or pharmaceutically acceptable salt, solvate, hydrate, stereoisomer, or prodrug thereof of claim 7, wherein:
W⁷ and W⁸ are each independently a bond or substituted or unsubstituted alkylene;
L^{a3} is a bond;
R¹⁰ and R¹¹ are each independently H, substituted or unsubstituted alkyl or R¹⁰ and R¹¹ together with the nitrogen atom to which they are connected form a substituted or unsubstituted heterocycloalkyl;
B⁵ is a bond;
B⁶ and B⁷ are each independently a bond or substituted or unsubstituted alkylene;
L⁸ is a bond;
L⁹ is a bond, —O(C═O)—, —(C═O)O—, or —C(═O)—;
L¹⁰ is a bond, —O(C═O)—, —(C═O)O—, or —C(═O)—; and
R⁷, R⁸, and R⁹ are each independently H or substituted or unsubstituted C₁-C₃₀ alkyl.

9. The cationic lipid or pharmaceutically acceptable salt, solvate, hydrate, stereoisomer, or prodrug thereof of claim 7, wherein:
W⁷ and W⁸ are each independently a bond or substituted or unsubstituted C₁-C₈ alkylene;
L^{a3} is a bond;
R¹⁰ and R¹¹ are each independently substituted or unsubstituted alkyl or R¹⁰ and R¹¹ together with the nitrogen atom to which they are connected form a substituted or unsubstituted heterocycloalkyl;
B⁵ is a bond;
B⁶ and B⁷ are each independently a bond or substituted or unsubstituted C₁-C₈ alkylene;
L⁸ is a bond;
L⁹ is —O(C═O)— or —(C═O)O—;
L¹⁰-O(C═O)— or —(C═O)O—; and
R⁷, R⁸, and R⁹ are each independently substituted or unsubstituted C₁-C₂₀ alkyl.

10. The cationic lipid or pharmaceutically acceptable salt, solvate, hydrate, stereoisomer, or prodrug thereof of claim 7, wherein:
W⁷ and W⁸ are each independently a bond or substituted or unsubstituted C₂-C₄ alkylene;
L^{a3} is a bond;
R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 3- to 8-membered heterocycloalkyl;
B⁵ is a bond;
B⁶ and B⁷ are each independently a bond or substituted or unsubstituted C₂-C₄ alkylene;
L⁸ is a bond;
L⁹ is —O(C═O)— or —(C═O)O—;
L¹⁰-O(C═O)— or —(C═O)O—;
R⁷ is H or methyl; and
R⁸, and R⁹ are each independently substituted or unsubstituted C₁-C₂₀ alkyl.

11. The cationic lipid or pharmaceutically acceptable salt, solvate, hydrate, stereoisomer, or prodrug thereof of claim 7, wherein:
W⁷ and W⁸ are each independently a bond or unsubstituted C₂-C₄ alkylene;
L^{a3} is a bond;
R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 5- to 6-membered heterocycloalkyl;
B⁵ is a bond;
B⁶ and B⁷ are each independently a bond or unsubstituted C₂-C₄ alkylene;
L⁸ is a bond;
L⁹ is —O(C═O)— or —(C═O)O—;
L¹⁰ is —O(C═O)— or —(C═O)O—;
R⁷ is H or methyl; and
R⁸ and R⁹ are each independently substituted or unsubstituted C₁-C₂₀ alkyl.

12. (canceled)

13. The cationic lipid or pharmaceutically acceptable salt, solvate, hydrate, stereoisomer, or prodrug thereof of claim 7, wherein the cationic lipid is: 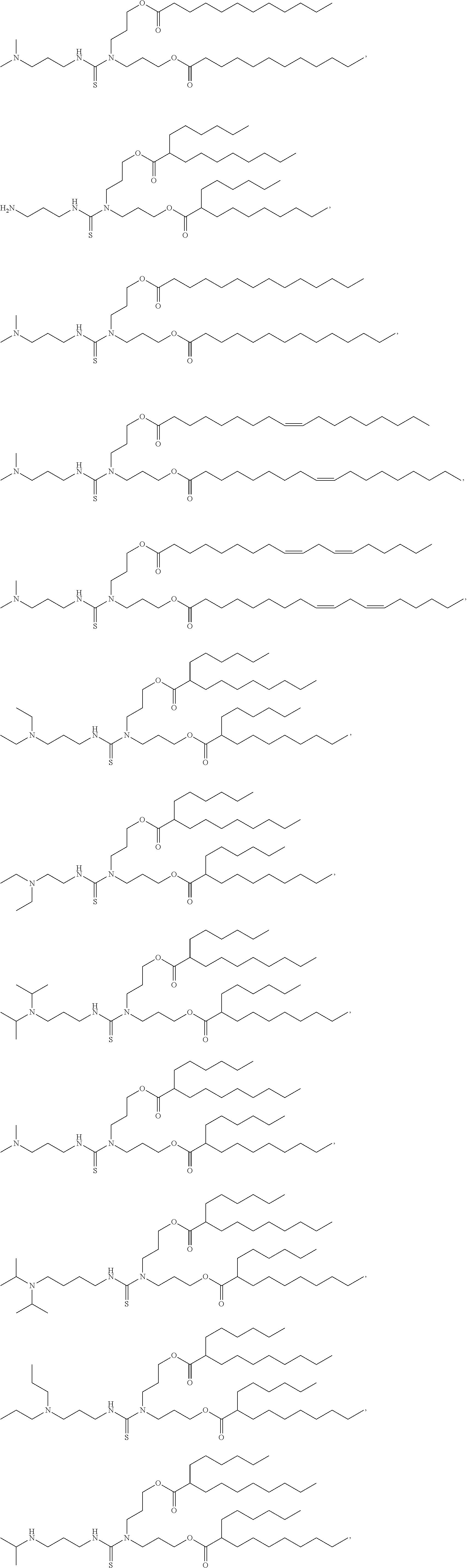 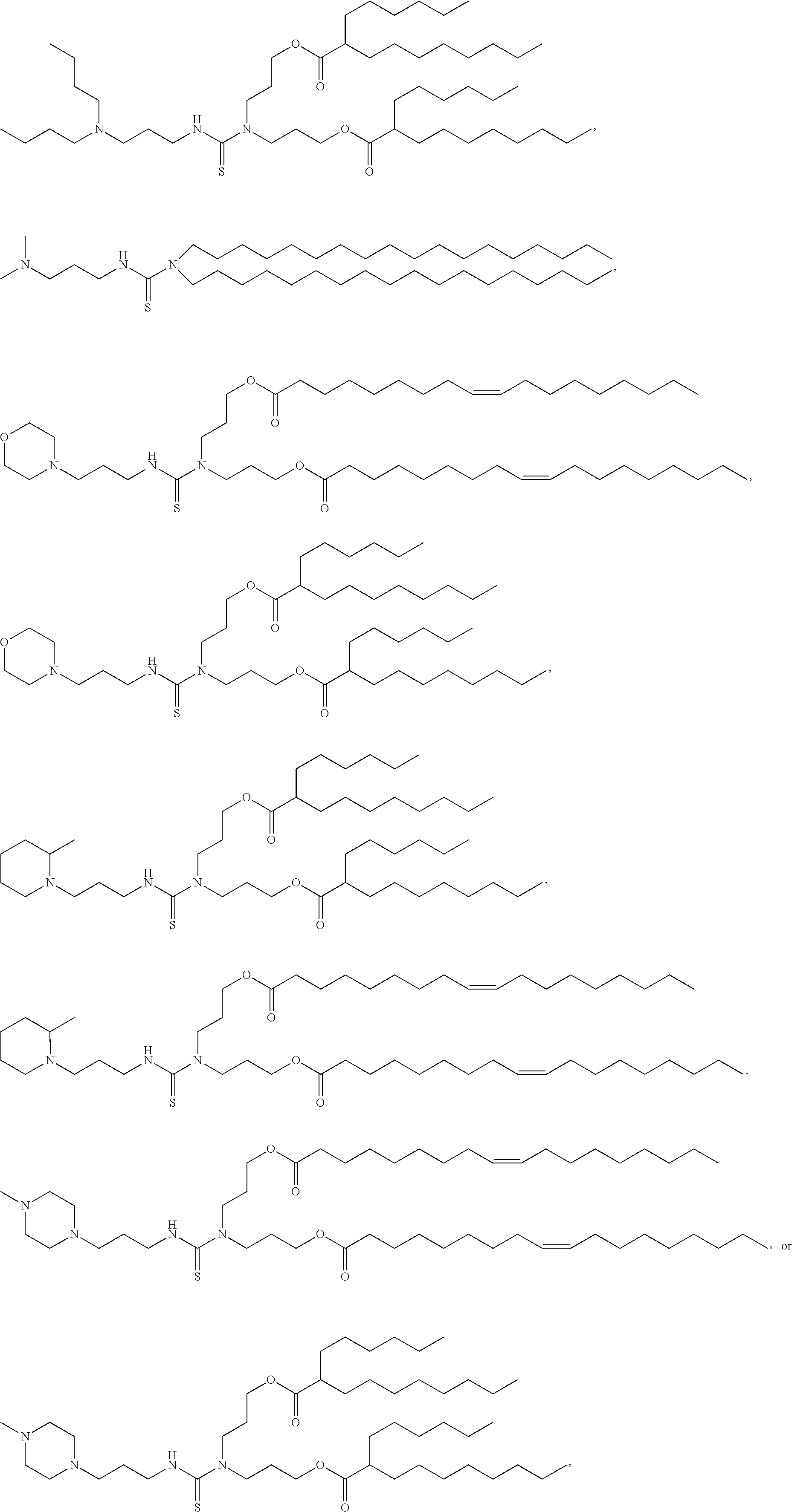
or a pharmaceutically acceptable salt thereof.

14. A cationic lipid, wherein:
(a) the cationic lipid is of formula (III): 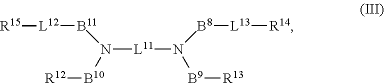
or a pharmaceutically acceptable salt, solvate, hydrate, stereoisomer, or prodrug thereof, wherein:
L¹¹ is 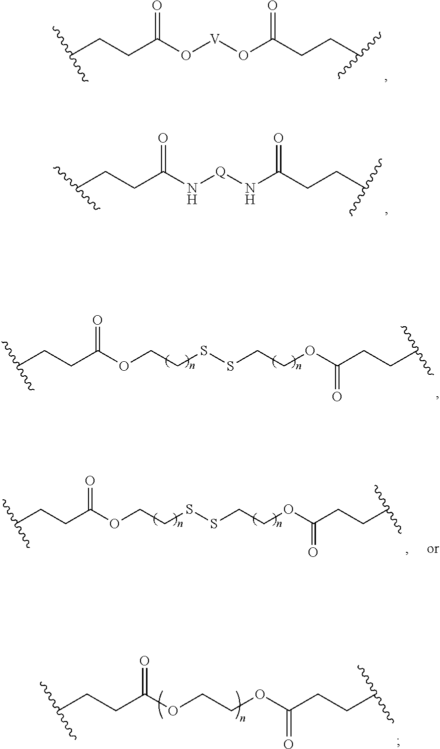
Q is substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene;
V is substituted or unsubstituted alkylene, substituted or unsubstituted cycloalkylene, substituted or unsubstituted arylene;
B⁸, B⁹, B¹⁰, and B¹¹ are each independently a bond, substituted or unsubstituted alkylene, or substituted or unsubstituted heteroalkylene;
L¹² is a bond, —O(C═O)—, —(C═O)O—, —O(C═O)O—, —C(═O)—, —O—, —O(CR²¹⁰R²¹¹)ₛO—, —S—, —C(═O)S—, —SC(═O)—, —NR²¹⁰C(═O)—, —C(═O)NR²¹⁰—, —NR²¹⁰C(═O)NR²¹¹—, —NR²¹⁰C(═S)—, —C(═S)NR²¹⁰—, —NR²¹⁰C(═S)NR²¹¹—, —OC(═O)NR²¹⁰—, —NR²¹⁰C(═O)O—, —SC(═O)NR²¹⁰— or —NR²¹⁰C(═O)S—;
L¹³ is a bond, —O(C═O)—, —(C═O)O—, —O(C═O)O—, —C(═O)—, —O—, —O(CR³¹⁰R³¹¹)ₛO—, —S—, —C(═O)S—, —SC(═O)—, —NR³¹⁰C(═O)—, —C(═O)NR³¹⁰—, —NR³¹⁰C(═O)NR³¹¹—, —NR³¹⁰C(═S)—, —C(═S)NR³¹⁰—, —NR³¹⁰C(═S)NR³¹—, —OC(═O)NR³¹⁰—, —NR³¹⁰C(═O)O—, —SC(═O)NR³¹⁰— or —NR¹⁰C(═O)S—;
R¹² is H, —OR^{12A}, —SR^{12A}, —NR^{12A}, —CN, —(C═O)R^{12A}, O(C═O)R^{12A}—(C═O)OR^{12A}, —NR^{12A}(C═O)—R^{12B}, —(C═O)NR^{12A}R^{12B};
R¹³ is H, —OR¹³A, —SR^{13A}, —NR^{13A}, —CN, —(C═O)R^{13A}, —O(C═O)R^{13A}, —(C═O)OR^{13A}, —NR^{13A}(C═O)—R^{13B}, —(C═O)NR^{13A}R^{13B};
R¹⁴ and R¹⁵ are each independently substituted or unsubstituted C₂-C₃₀ alkyl, or substituted or unsubstituted 2- to 30-membered heteroalkyl;
R^{12A}, R^{12B}, R^{13A}, and R^{13B} are each independently H, substituted or unsubstituted C₁-C₂₀ alkyl, or substituted or unsubstituted 2- to 20-membered heteroalkyl;
each R²¹⁰, R²¹¹, R³¹⁰, and R³¹¹ is independently H, substituted or unsubstituted C₁-C₁₂ alkyl, or substituted or unsubstituted 2- to 12-membered heteroalkyl;
each n is independently an integer from 0 to 8; and
each s is independently an integer from 1 to 4; or
(b) the cationic lipid is of formula (IV): 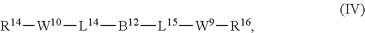
or a pharmaceutically acceptable salt, solvate, hydrate, stereoisomer, or prodrug thereof,
wherein:
B¹² is —W⁷-L^{a3}-W⁸—:
W⁷ and W⁸ are each independently a bond, substituted or unsubstituted C₁-C₁₂ alkylene, or substituted or unsubstituted 2- to 12-membered heteroalkylene:
L^{a3} is a bond, —S—S—, —O—(CH₂O)ₘ—, 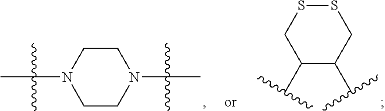 [SMILES: *N1CCN(*)CC1 ; *C1CSSCC1*]
W⁹ and W¹⁰ are each independently a bond, substituted or unsubstituted C₁-C₁₂ alkylene, substituted or unsubstituted 2- to 12-membered heteroalkylene, substituted or unsubstituted cycloalkylene, substituted or unsubstituted heterocycloalkylene, or any combination thereof;
L¹⁴ is a bond, —O(C═O)—, —(C═O)O—, —O(C═O)O—, —C(O)—, —O—, —O(CR⁴¹⁰R⁴¹¹)ₛO—, —S—, —C(═O)S—, —SC(═O)—, —NR⁴¹⁰C(═O)—, —C(═O)NR⁴¹⁰—, —NR⁴¹⁰C(═O)NR⁴¹¹—, —NR⁴¹⁰C(═S)—, —C(═S)NR⁴¹⁰—, —NR⁴¹⁰C(═S)NR⁴¹¹—, —OC(═O)NR⁴¹⁰—, —NR⁴¹⁰C(═O)O—, —SC(═O)NR⁴¹⁰— or —NR⁴¹⁰C(═O)S—;
L¹⁵ is a bond, —O(C═O)—, —(C═O)O—, —O(C═O)O—, —C(O)—, —O—, —O(CR⁵¹⁰R⁵¹¹)ₛO—, —S—, —C(═O)S—, —SC(═O)—, —NR⁵¹⁰C(═O)—, —C(═O)NR⁵¹⁰—, —NR⁵¹⁰C(═O)NR⁵¹¹—, —NR⁵¹⁰C(═S)—, —C(═S)NR⁵¹⁰—, —NR⁵¹⁰C(═S)NR⁵¹¹—, —OC(═O)NR⁵¹⁰—, —NR⁵¹⁰C(═O)O—, —SC(═O)NR⁵¹⁰— or —NR⁵¹⁰C(═O)S—,
R¹⁶ and R¹⁷ are each independently 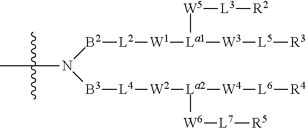
a fragment of cationic lipid of formula (I), 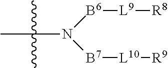
a fragment of cationic lipid of formula (II), 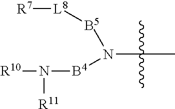
fragment of cationic lipid of formula (II), 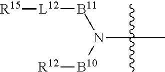
a fragment of cationic lipid of formula (III), or 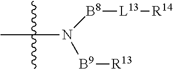
a fragment of cationic lipid of formula (III);
each R⁴¹⁰, R⁴¹¹, R⁵¹⁰, and R⁵¹¹ is independently H, substituted or unsubstituted C₁-C₁₂ alkyl, or substituted or unsubstituted 2- to 12-membered heteroalkyl;
each m is independently an integer from 0 to 8; and
each s is independently an integer from 1 to 4.

15-17. (canceled)

18. The cationic lipid or pharmaceutically acceptable salt, solvate, hydrate, stereoisomer, or prodrug thereof of claim 14, wherein the cationic lipid is: 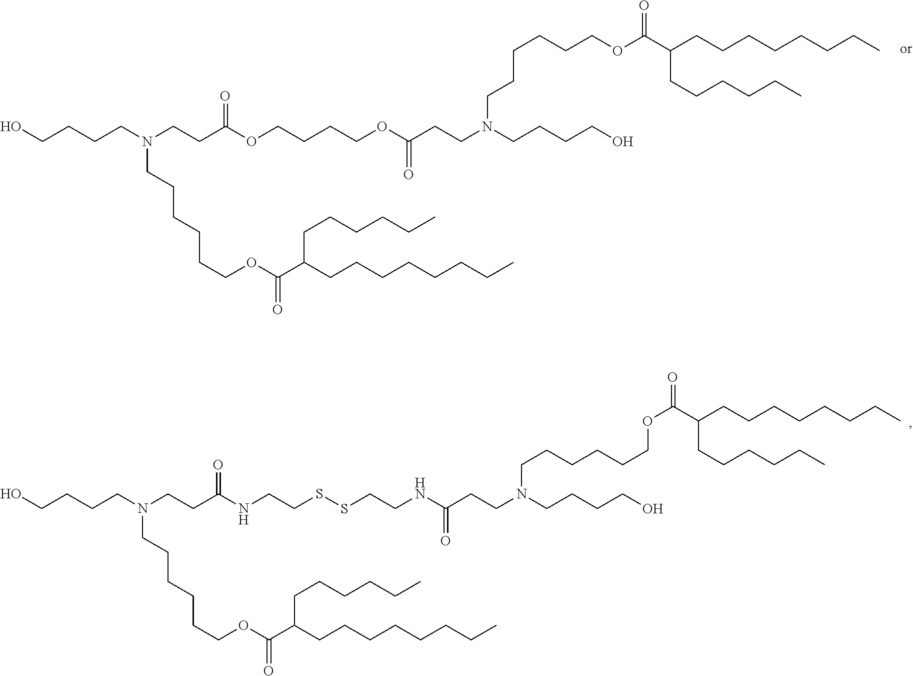 [SMILES: CCCCCCCCC(CCCCCC)C(=O)OCCCCCCN(CCCCO)CCC(=O)OCCCCOC(=O)CCN(CCCCO)CCCCCCOC(=O)C(CCCCCC)CCCCCCCC ; CCCCCCCCC(CCCCCC)C(=O)OCCCCCCN(CCCCO)CCC(=O)NCCSSCCNC(=O)CCN(CCCCO)CCCCCCOC(=O)C(CCCCCC)CCCCCCCC]
or a pharmaceutically acceptable salt thereof.

19-23. (canceled)

24. The cationic lipid or pharmaceutically acceptable salt, solvate, hydrate, stereoisomer, or prodrug thereof of claim 14, wherein the cationic lipid is: 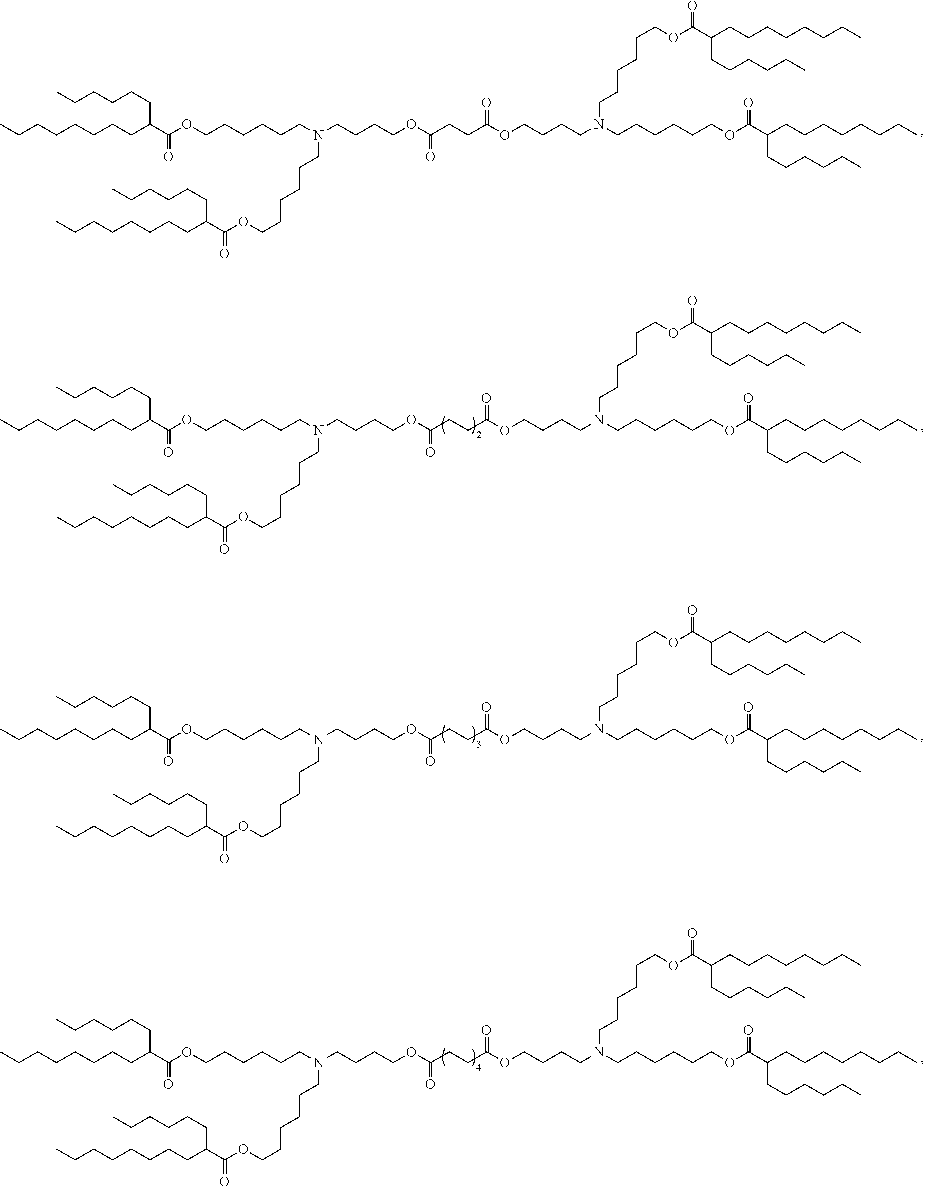 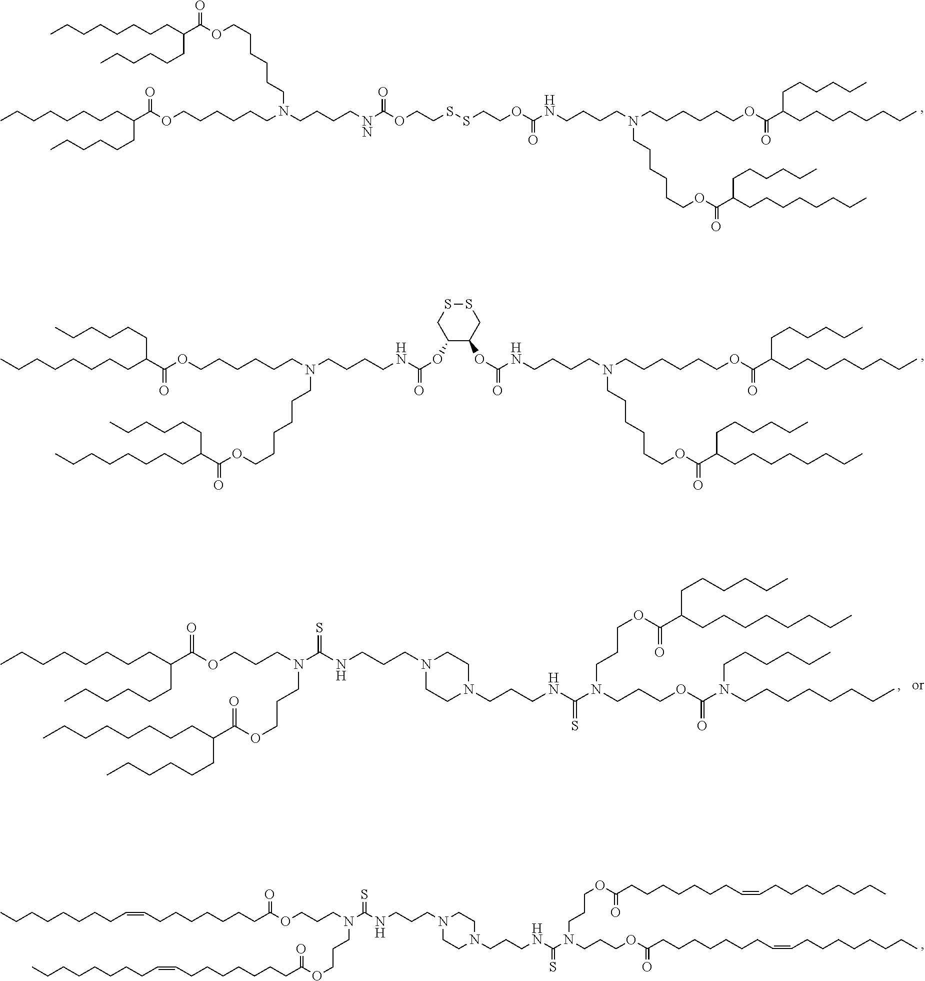
or a pharmaceutically acceptable salt thereof.

25. A lipid nanoparticle comprising the cationic lipid or pharmaceutically acceptable salt, solvate, hydrate, stereoisomer, or prodrug thereof of claim 1.

26. The lipid nanoparticle of claim 25, wherein the lipid nanoparticle further comprises a non-cationic lipid.

27-29. (canceled)

30. The lipid nanoparticle of claim 25, wherein the lipid nanoparticle further comprises a therapeutic agent.

31-33. (canceled)

34. A method for the in vivo delivery of a therapeutic agent, the method comprising administering the lipid nanoparticle of claim 30 to a mammal.

35. (canceled)

36. A method of treating a disease in a mammal in need thereof, the method comprising administering to the mammal a therapeutically effective amount of the lipid nanoparticle of claim 30.

37-39. (canceled)
